(19) United States
(12) Patent Application Publication
(10) Pub. No.: US 2022/0185793 A1
(43) Pub. Date: Jun. 16, 2022

(54) COMPOUNDS FOR THE TREATMENT OF ONCOVIRUS INDUCED CANCER AND METHODS OF USE THEREOF

(71) Applicant: Cellestia Biotech AG, Basel (CH)

(72) Inventors: Rajwinder Lehal, Zürich (CH); Guido Bold, Gipf-Oberfrick (CH); Charlotte Urech, Basel (CH); Vincent Zoete, Morges (CH)

(21) Appl. No.: 17/602,953

(22) PCT Filed: Apr. 9, 2020
(86) PCT No.: PCT/EP2020/060149

§ 371 Date: Oct. 11, 2021

(30) Foreign Application Priority Data
Apr. 10, 2019 (EP) 19168505.6

(51) Int. Cl. C07D 401/14; C07D 401/12; C07D 413/12; C07D 213/75; C07D 213/38; C07D 417/14

(52) U.S. Cl. CPC C07D 401/14; C07D 401/12; C07D 417/14; C07D 213/75; C07D 213/38; C07D 413/12

(57) ABSTRACT

The pesent invention relates to compounds of formula (I) pharmaceutically-acceptable salts, hydrates, solvates, or stereoisomers thereof and their use for the prevention and treatment of oncovirus induced cancer in a subject.

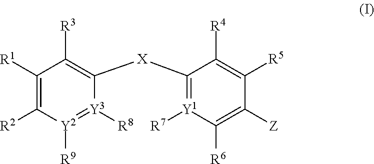

### FIELD OF THE INVENTION

The pesent invention relates to compounds of formula (I)

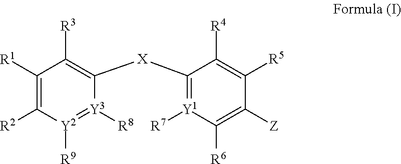

pharmaceutically-acceptable salts, hydrates, solvates, or stereoisomers thereof and their use for the prevention and treatment of oncovirus induced cancer in a subject.

### BACKGROUND OF THE INVENTION

Cancer is the leading cause of death. About 14 million new cases of cancers are diagnosed every year and leading to 8.8 million cancer related deaths. In addition to genetic and environmental factors, oncoviruses are known to account for about 12-15% of all human cancers. Due to a complex molecular interplay between virus and their host, lack of appropriate preclinical animal models the management of virus-induced cancers remains a high unmet medical need.

There are at least seven oncoviruses known to cause human cancers. The list includes Epstein Barr virus (EBV), Kaposi's sarcoma herpesvirus (KSHV), Human papillomavirus (HPV), Hepatitis B virus (HBV), Hepatitis C virus (HCV), Human T Cell lymphotrophic virus type 1 (HTLV-1) and Human Immunodeficiency Virus (HIV). Oncoviruses trigger oncogenic transformation of normal cells by hijacking cellular mitogenic pathway as well as evading host immune surveillance (Mesri E A, Feitelson M A, Munger K. 2014. Human viral oncogenesis: A cancer hallmarks analysis. Cell Host & Microbe 15: 266-282).

Common malignancies associated with oncoviruses are as follows:

Epstein Barr Virus associated cancers:

EBV is a double stranded DNA virus and belongs to γ-herpesvirus family of viruses. EBV is primarily known to infect B cells, but in certain instances is also known to infect epithelial cells. Under certain conditions such as immune suppression, EBV viral genes get activated leading to oncogenic transformation of the infected host cells. EBV is associated with several lymphoid malignancies that include Burkitt lymphoma, and classical Hodgkin lymphoma. EBV is also linked with immunodeficiency-associated lymphoproliferative disorders such as post-transplant lymphoproliferative disorder (PTLD), non-Hodgkin lymphoma (NHL).

Examples of EBV associated epithelial tumors consist of Nasopharyngeal Carcinoma (NPC) and gastric tumors.

Kaposi's Sarcoma Herpesvirus (KSHV) driven cancers:

KSHV also known as human herpes virus 8 (HHV-8) also belongs to γ-herpesvirus family of viruses. KSHV is known to cause three main human cancers, namely Kaposi's Sarcoma (KS), Primary Effusion Lymphoma (PEL) and Multicentric Castleman's disease (MCD). All three cancers occur primarily in the context of immune deficiency and/or HIV infection (Dittmer D P, Damania B. 2016. Kaposi sarcoma-associated herpesvirus: immunobiology, oncogenesis, and therapy. The Journal of clinical investigation 126: 3165-3175).

Human Papillomavirus (HPV) associated cancers:

HPV is one of the leading causes of virus—induced cancers. This double stranded DNA virus predominantly infects epithelial cells and thereby is an established cause for cervical carcinomas, vulvar, vaginal and oral carcinomas (Mesri E A, Feitelson M A, Munger K. 2014. Human viral oncogenesis: A cancer hallmarks analysis. Cell Host & Microbe 15: 266-282). Hepatitis B and Hepatitis C virus (HBV and HCV) induced human carcinomas: HBV and HCV are major etiological agents for hepatocellular carcinomas (Mesri E A, Feitelson M A, Munger K. 2014. Human viral oncogenesis: A cancer hallmarks analysis. Cell Host & Microbe 15: 266-282).

Human T Cell Lymphotropic Virus-1 (HTLV-1): HTLV-1 is known to cause adult T-cell Leukemia (Mesri E A, Feitelson M A, Munger K. 2014. Human viral oncogenesis: A cancer hallmarks analysis. Cell Host & Microbe 15: 266-282).

Oncoviruses deploy a variety of mechanisms to trigger oncogenic transformation of infected cells. This includes hijacking and activation of cancer-causing cellular pathways, chronic inflammation and induction of genomic instability. Almost all of the oncoviruses are known to express oncogenic viral homologs of host proteins and thereby driving cell survival, proliferation and evasion of immune surveillance. Infection by HBV and HCV is known to cause chronic inflammation of the liver and thereby promote hepatocellular carcinomas (Mesri E A, Feitelson M A, Munger K. 2014. Human viral oncogenesis: A cancer hallmarks analysis. Cell Host & Microbe 15: 266-282).

Several therapeutic strategies have been developed to treat virus-induced human cancers. These can be broadly subdivided into two categories, 1) agents targeting viral oncogenes and 2) agents targeting host proteins. The development of prophylactic vaccine against HPV and several nucleoside analogs represent examples of anti-viral therapies. In addition, several drugs targeting cellular oncogenes have also been used to address virus-induced malignancies, such as inhibitors of PI3K/mTOR signaling, PDGFR, c-kit. However, treatment of oncovirus induced human cancers still poses a major challenge and there is a high need to develop novel therapeutic agents.

### SUMMARY OF THE INVENTION

The present invention relates to compounds according to formula (I)

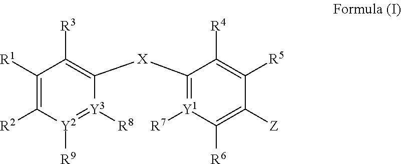

pharmaceutically-acceptable salts, hydrates, solvates, or stereoisomers thereof,

wherein X is selected from CH₂, CF₂, CHF, CO, CHOH, CHO(C₁-C₃) alkyl, NH, N(C₁-C₃ alkyl), S, SO and O;

wherein Y¹, Y² and Y³ are each independently selected from N and C;

wherein Z is NR¹⁰R¹¹;

wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and

R¹¹ is selected from C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl;

wherein R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

wherein R³ is selected from H, halogen, C₁-C₆ alkyl, C₁-C₆ heteroalkyl, C₃-C₁₂ cycloalkyl and C₃-C₁₂ heterocyclyl;

wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆-S-alkyl, C₁-C₆ alkyl, C₁-C₆ heteroalkyl, C₃-C₁₂ cycloalkyl and C₃-C₁₂ heterocyclyl;

wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;

wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;

wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²_{R}¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl.

The present invention also relates to a pharmaceutical composition comprising the compounds of formula (I) and pharmaceutically-acceptable salts, hydrates, solvates, or stereoisomers thereof and the compounds of formula (I) and pharmaceutically-acceptable salts, hydrates, solvates, or stereoisomers thereof and pharmaceutical compositions thereof for use in a method for the prevention or treatment of oncovirus induced cancer in a subject. The present invention is useful in methods for preventing and treating oncovirus induced cancer.

### DESCRIPTION OF THE FIGURES

FIG. 1 shows anti-proliferative effect of compounds on EBV positive human lymphoma cell line HG-3. Cells were treated with compounds for 72 hours and effect on proliferation was quantified using Alamar blue readout.

FIG. 2 shows anti-proliferative effect of compounds on EBV positive human lymphoma cell line HG-3. Cells were treated with compounds for 72 hours and effect on proliferation was quantified using Prestoblue readout.

FIG. 3 shows effect of compounds on BMI1 (EBV target gene) in human B cells. EBV positive human B LCL070903 cells were treated with 10 μM of the mentioned compounds for 48 hours. Following treatment, total RNA was extracted and mRNA expression analysed by qRT-PCR. Data shows that 4-(4-(tert-butyl)phenoxy)-3-fluoro-N-(pyridin-3-ylmethyl)aniline, 6-([1,1′-biphenyl]-4-yloxy)-N-(pyridin-3-ylmethyl)pyridin-3-amin), 6-((6-phenylpyridin-3-yl)oxy)-N-(pyridin-3-ylmethyl)pyridin-3-amin), 6-([1,1′-biphenyl]-4-yloxy)-4-methyl-N-(pyridin-3-ylmethyl)pyridin-3-amine, 6-([1,1′-biphenyl]-4-yloxy)-2-methyl-N-(pyridin-3-ylmethyl)pyridin-3-amine, 4-(4-cyclohexylphenoxy)-3-fluoro-N-(pyridin-3-ylmethyl)aniline, 6-((4′-fluoro-[1,1′-biphenyl]-4-yl)oxy)-N-(pyridin-3-ylmethyl)pyridin-3-amine, 3-fluoro-4-(4-(pyridin-2-yl)phenoxy)-N-(pyridin-3-ylmethyl)aniline, N-(pyridin-3-ylmethyl)-6-(4-(thiazol-5-yl)phenoxy)pyridin-3-amine, 3-fluoro-4-(4-(pyridin-3-yl)phenoxy)-N-(pyridin-3-ylmethyl)aniline, (4′-fluoro-[1,1′-biphenyl]-4-yl)(4-((pyridin-3-ylmethyl)-amino)phenyl)methanol), 4-((4′-fluoro-[1,1′-biphenyl]-4-yl)(methoxy)methyl)-N-(pyridin-3-ylmethyl)anilin, 4-((4′-fluoro-[1,1′-biphenyl]-4-yl)methyl)-N-(pyridin-3-ylmethyl)aniline, 4-((2,2′-dimethyl-[1,1′-biphenyl]-4-yl)oxy)-N-(pyridin-3-ylmethyl)aniline and 6-((2,2′-dimethyl-[1,1′biphenyl]-4-yl)oxy)-N-(pyridin-3-ylmethyl)pyridin-3-amine) have enhanced potency in downregulating EBV pathway in human LCL070903 cells compared with 6-(4-tert-butylphenoxy)pyridin-3-amine.

FIG. 4 shows effect of compounds on BMI1 (EBV target gene) in human cancer B cells. EBV positive human B HG-3 cells were treated with 10 μM of the mentioned compounds for 48 hours. Following treatment, total RNA was extracted and mRNA expression analysed by qRT-PCR. Data shows that 4-(4-(tert-butyl)phenoxy)-3-fluoro-N-(pyridin-3-ylmethyl)aniline, 6-((6-phenylpyridin-3-yl)oxy)-N-(pyridin-3-ylmethyl)pyridin-3-amine, 6-([1,1′-biphenyl]-4-yloxy)-4-methyl-N-(pyridin-3-ylmethyl)pyridin-3-amine, 6-([1,1′-biphenyl]-4-yloxy)-2-methyl-N-(pyridin-3-ylmethyl)pyridin-3-amine, (4-(4-cyclohexylphenoxy)-3-fluoro-N-(pyridin-3-ylmethyl)aniline, 6-((4′-fluoro-[1,1′-biphenyl]-4-yl)oxy)-N-(pyridin-3-ylmethyl)pyridin-3-amine, 3-fluoro-4-(4-(pyridin-2-yl)phenoxy)-N-(pyridin-3-ylmethyl)aniline, N-(pyridin-3-ylmethyl)-6-(4-(thiazol-5-yl)phenoxy)pyridin-3-amine, 3-fluoro-4-(4-(pyridin-3-yl)phenoxy)-N-(pyridin-3-ylmethyl)aniline, (4′-fluoro-[1,1′-biphenyl]-4-yl)(4-((pyridin-3-ylmethyl)-amino)phenyl)methanol), 4-((4′-fluoro-[1,1′-biphenyl]-4-yl)(methoxy)methyl)-N-(pyridin-3-ylmethyl)aniline, 4-((4′-fluoro-[1,1′-biphenyl]-4-yl)methyl)-N-(pyridin-3-ylmethyl)aniline, 4-((2,2′-dimethyl-[1,1′-biphenyl]-4-yl)oxy)-N-(pyridin-3-ylmethyl)aniline, 6-((2,2′-dimethyl-[1,1′-biphenyl]-4-yl)oxy)-N-(pyridin-3-ylmethyl)pyridin-3-amine have enhanced potency in downregulating EBV pathway in human HG-3 cells compared with 6-(4-tert-butylphenoxy)pyridin-3-amine.

### DETAILED DESCRIPTION OF THE INVENTION

The following are definitions of terms used in the present application. The initial definition provided for a group or term herein applies to that group or term throughout the description and the claims, individually or as part of another group, unless otherwise indicated.

The term “alkyl” as used herein refers to a saturated straight or branched chain group of carbon atoms derived from an alkane by the removal of one hydrogen atom. C₁-C₃ alkyl comprises for example methyl, ethyl, n-propyl, i-propyl. C₁-C₆ alkyl comprises for example methyl, ethyl, n-propyl, i-propyl, n-butyl, i-butyl, tert-butyl, n-pentyl, and n-hexyl. C₃-C₆ alkyl comprises for example n-propyl, i-propyl, n-butyl, i-butyl, tert-butyl, n-pentyl or n-hexyl. C₂-C₆ alkyl comprises for example ethyl, n-propyl, i-propyl, n-butyl, i-butyl, tert-butyl, n-pentyl, and n-hexyl.

The term “heteroalkyl” as used herein refers to an alkyl radical as defined herein wherein one, two, three or four hydrogen atoms have been replaced with a substituent independently selected from the group consisting of OR^{a}, C(O)OR^{a}, NR^{b}R^{c}, C(O)NR^{b}R^{c}, S(O)ₙR^{d} (wherein n is an integer from 0 to 2) and halogen, with the understanding that the point of attachment of the heteroalkyl radical is through a carbon atom, wherein R^{a} is H, C₁-C₃ alkylcarbonyl, C₁-C₃ alkyl, or C₃₋₇ cycloalkyl; R^{b} and R^{c} are each independently H, C₁-C₃ alkylcarbonyl, C₁-C₃ alkyl, or C₃₋₇ cycloalkyl; and when n is 0, R^{d} is H, C₁-C₃ alkyl or C₃₋₇ cycloalkyl, and when n is 1 or 2, R^{d} is C₁-C₃ alkyl or C₃₋₇ cycloalkyl. Preferably. the term “heteroalkyl” as used herein refers to an alkyl radical or an alkanediyl radical as defined herein wherein one, two, three or four hydrogen atoms have been replaced with a substituent independently selected from the group consisting of OH, NH₂ and halogen, more preferably selected from the group consisting of OH and NH₂. Representative examples of C₁-C₆ heteroalkyl include, but are not limited to, 2-hydroxyethyl, 2-hydroxypropyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 1-hydroxy-2-methylpropyl, 3-hydroxy-1-(2-hydroxyethyl)-propyl, 2-hydroxy-1-methylpropyl, 1,1,1-trifluoroethyl, 1,1,1-trifluoromethyl, 2,2,3,3-tetrafluoropropyl. Representative examples of C₂-C₆ heteroalkyl include, but are not limited to, 2-hydroxyethyl, 2-hydroxypropyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 1-hydroxy-2-methylpropyl, 3-hydroxy-1-(2-hydroxyethyl)-propyl, 2-hydroxy-1-methylpropyl, 1,1,1-trifluoroethyl, 2,2,3,3-tetrafluoropropyl.

The term “C₁-C₃ cyanoalkyl” as used herein refers to an alkyl radical as defined herein wherein one, two or three hydrogen atoms have been replaced with a CN group. C₁-C₃ cyanoalkyl is preferably cyanomethyl.

The term “C₂-C₆ alkenyl” as used herein refers to straight or branched chain hydrocarbon groups having 2 to 10 carbon atoms and at least one double bond.

The term “C₂-C₆ alkynyl” as used herein refers to straight or branched chain hydrocarbon groups having 2 to10 carbon atoms and at least one triple bond.

The term “C₃-C₁₂ cycloalkyl ” as used herein refers to a monovalent saturated monocyclic or bicyclic hydrocarbon group, preferably a monovalent saturated monocyclic goup of 3-12, preferably 3-7 carbons, respectively derived from a cycloalkane by the removal of a single hydrogen atom. A preferred C₃-C₁₂ cycloalkyl group is thus a “C₃-C₇ cycloalkyl” group which includes cyclopropyl, cyclobutyl, cyclopentyl, cyclohexyl and cycloheptyl. The term “C₃-C₁₂ cycloalkyl” and “C₃-C₇ cycloalkyl” “as used herein also includes cycloalkyl groups that comprise a C₁₋₃ alkyl radical. Examples of such groups comprise cyclopropylmethyl, 2-cyclopropylethyl, cyclobutylmethyl, 2-cyclobutylethyl, cyclopentylmethyl, 2-cyclopentylethyl.

The term “C₁-C₆ alkoxy” as used herein refers to a radical —OR where R is a C₁-C₆ alkyl as defined herein. Examples are methoxy, ethoxy, propoxy, butoxy.

The term “C₁-C₆ alkylamino” as used herein refers to a radical —NRR′ where one of R and R′ represent a C₁-C₆ alkyl or cycloalkyl group as defined herein. Representative examples include, but are not limited to methylamino, ethylamino.

The term “C₁-C₆ dialkylamino” as used herein refers to a radical —NRR′ where R and R′ independently represent a C₁-C₆ alkyl or cycloalkyl group as defined herein. Representative examples include, but are not limited to dimethylamino, methylethylamino, di(1-methylethyl)amino, (methyl)(hydroxyethyl)amino, (cyclohexyl)(methyl)amino, (cyclohexyl)(ethyl)amino, (cyclohexyl)(propyl)amino.

The term “C₁-C₃ alkanoyl” as used herein refers to a radical —CO—C₁-C₃ alkyl wherein the C₁-C₃ alkyl group is as defined herein.

The term “(C₁-C₆)alkyl carboxy” as used herein refers to a radical —RC(O)OH, wherein R is a C₁-C₆ alkyl group, wherein the C₁-C₆ alkyl group is as defined herein.

The term “C₁-C₃ alkoxycarbonyl” as used herein refers to radicals —C(O)O C₁-C₃ alkyl, —C₁-C₃ alkyl C(O)O C₁-C₃ alkyl and —OC(O) C₁-C₃ alkyl and is preferably —C(O)O C₁-C₃ alkyl wherein the C₁-C₃ alkyl group is as defined herein.

The term “aryl” as used herein refers to a mono- or bicyclic carbocyclic ring system having one or two aromatic rings, and is preferably a monocyclic carbocyclic ring system having one aromatic ring. The aryl group can also be fused to a cyclohexane, cyclohexene, cyclopentane, or cyclopentene ring or to a cyclohexane, cyclohexene, cyclopentane, or cyclopentene ring comprising a carbonyl group. The aryl groups of this invention can be optionally substituted as further described below. A preferred aryl group and optionally substituted aryl group, respectively of this invention is a phenyl group or substituted phenyl group. Substituents can be e.g. NH₂, N(C₁-C₆ alkyl)₂, NH(C₁-C₆ alkyl), OH, O(C₁-C₆) alkyl, C₁-C₆ alkyl, C₁-C₁₀ heteroalkyl, halogen, CN, CF₃, C₃-C₁₂ cycloalkyl, CHO, carbonyl(C₁-C₁₀ alkyl), C₁-C₃ alkoxycarbonyl, or (C₁-C₁₀ alkyl)carbonyl(C₁-C₁₀ alkyl). Preferably such aryl groups are unsubstituted unless otherwise indicated herein.

The term “heteroaryl” as used herein refers to substituted and unsubstituted aromatic 5-, or 6-membered monocyclic groups and 9- or 10-membered bicyclic groups, preferably a substituted and unsubstituted aromatic 5-, or 6- membered monocyclic group, which have at least one heteroatom (O, S or N), preferably one to four N or one to two N and one O, in at least one of the ring(s). Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic group may contain only carbon atoms and may be saturated, partially saturated, or unsaturated. Heteroaryl groups must include at least one fully aromatic ring but the other fused ring or rings may be aromatic or non-aromatic. The heteroaryl group may be attached at any available nitrogen or carbon atom of any ring. Heteroaryl groups of this invention can be optionally substituted as further described below. Usually, a heteroaryl group and optionally substituted heteroaryl group, respectively of this invention is selected from the group consisting of substituted and/or unsubstituted aromatic 5-, or 6-membered monocyclic groups, which have at least one heteroatom (O, S or N), preferably one to four N or one to two N and one O, in the ring. A preferred heteroaryl group is an optionally substituted heteroaryl group selected from the group consisting of an optionally substituted pyridinyl group, an optionally substituted pyrimidinyl group, an optionally substituted di- or triazole group, an optionally substituted thiazole group, an optionally substituted oxazole group, an optionally substituted oxadiazole group and an optionally substituted imidazole group. Most preferably an optionally substituted pyridinyl group, an optionally substituted pyrimidinyl group, an optionally substituted tetrazole, an optionally substituted oxadiazole group, and/or an optionally substituted imidazole group, is used as heteroaryl group in the present invention, or an optionally substituted pyridinyl group, an optionally substituted pyrimidinyl group, an optionally substituted thiazole, an optionally substituted oxazole group and/or an optionally substituted imidazole group . Substituents can be e.g. NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably NH₂, C₁-C₆ alkyl, C₁-C₆ heteroalkyl, halogen, CN, C₃-C₁₂ cycloalkyl, C₃-C₁₂ heterocyclyl, more preferably NH₂, C₁-C₆ alkyl, C₁-C₆ heteroalkyl, halogen, even more preferably NH₂. Preferably such heteroaryl groups are unsubstituted unless otherwise indicated herein.

The term “C₃-C₁₂ heterocyclyl” as used herein means a saturated, monocyclic ring with 3 to 12, preferably 5 to 6 ring atoms which contains up to 3, preferably 1 or 2 heteroatoms selected independently from nitrogen, oxygen or sulfur, and wherein the remaining ring atoms being carbon atoms. Examples of such saturated heterocycles include [1,3]dioxanyl, [1,3]dioxolanyl, pyrrolidinyl, morpholinyl, piperazinyl, piperidinyl, oxazolidinyl, thiazolidinyl, azepanyl and the like. Preferably such heterocyclyl groups are unsubstituted unless otherwise indicated herein.

The terms “halo” or “halogen” as used herein refers to F, Cl, Br, or I and is preferably F, Cl, or Br, more preferably F.

The term ‘optionally substituted’ or ‘substituted’ means that the referenced group is substituted with one or more additional group(s), preferably with one additional group, individually and independently selected from the listed groups.

In one aspect the present invention provides compounds according to formula (I)

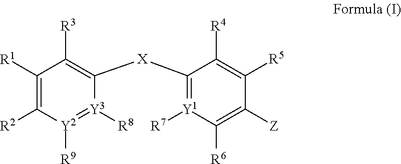

pharmaceutically-acceptable salts, hydrates, solvates, or stereoisomers thereof,

wherein X is selected from CH₂, CF₂, CHF, CO, CHOH, CHO(C₁-C₃) alkyl, NH, N(C₁-C₃ alkyl), S, SO and O;

wherein Y¹, Y² and Y³ are each independently selected from N and C;

wherein Z is NR¹⁰R¹¹;

wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and

R¹¹ is selected from C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³; wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl;

wherein R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C¹-C₆ alkyl-C(O)NR¹²R13, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

wherein R³ is selected from H, halogen, C₁-C₆ alkyl, C₁-C₆ heteroalkyl, C₃-C₁₂ cycloalkyl and C₃-C₁₂ heterocyclyl;

wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆ alkyl, C₁-C₆ heteroalkyl, C₃-C₁₂ cycloalkyl and C₃-C₁₂ heterocyclyl;

wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;

wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;

wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²_{R}¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl.

In a further aspect the present invention provides a pharmaceutical composition comprising compounds of formula (I)

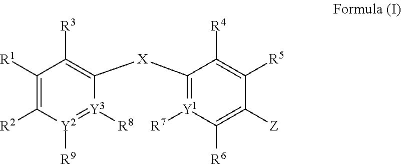

pharmaceutically-acceptable salts, hydrates, solvates, or stereoisomers thereof,

wherein X is selected from CH₂, CF₂, CHF, CO, CHOH, CHO(C₁-C₃) alkyl, NH, N(C₁-C₃ alkyl), S, SO and O;

wherein Y¹, Y² and Y³ are each independently selected from N and C;

wherein Z is NR¹⁰R¹¹;

wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and

R¹¹ is selected from C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³; wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl;

wherein R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

wherein R³ is selected from H, halogen, C₁-C₆ alkyl, C₁-C₆ heteroalkyl, C₃-C₁₂ cycloalkyl and C₃-C₁₂ heterocyclyl;

wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆ alkyl, C₁-C₆ heteroalkyl, C₃-C₁₂ cycloalkyl and C₃-C₁₂ heterocyclyl;

wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;

wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;

wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C;

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, and a pharmaceutically acceptable carrier.

In a further aspect the present invention provides compounds according to formula (I)

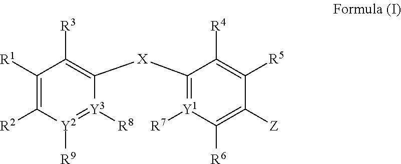

pharmaceutically-acceptable salts, hydrates, solvates, or stereoisomers thereof,

wherein X is selected from CH₂, CF₂, CHF, CO, CHOH, CHO(C₁-C₃) alkyl, NH, N(C₁-C₃ alkyl), S, SO and O;

wherein Y¹, Y² and Y³ are each independently selected from N and C;

wherein Z is NR¹⁰R¹¹;

wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and

R¹¹ is selected from C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³;

wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³; C(O)NR¹²R¹³; C₁-C₆ alkyl-C(O)NR¹²R¹³; C₁-C₆ alkoxy, C₁-C₃ alkoxycarbonyl, C₁-C₆ alkyl-NHCOR¹², C₁-C₃ alkanoyl, adamantyl and norbornyl;

wherein R² is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³; C(O)NR¹²R¹³; C₁-C₆ alkyl-C(O)NR¹²R¹³; C₁-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

wherein R³ is selected from H, halogen, C₁-C₆ alkyl, C₁-C₆ heteroalkyl, C₃-C₁₂ cycloalkyl and C₃-C₁₂ heterocyclyl;

wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆ alkyl, C₁-C₆ heteroalkyl, C₃-C₁₂ cycloalkyl and C₃-C₁₂ heterocyclyl;

wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;

wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;

wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl for use in a method for the prevention or treatment of oncovirus induced cancer in a subject.

The following embodiments, preferred embodiments and very preferred embodiments should apply without the need of repetition to all aspects and other embodiments.

In one embodiment X is selected from CH₂, CF₂, CHF, CHOH, CHO(C₁-C₃) alkyl and CO.

In a further embodiment X is selected from NH, N(C₁-C₃-alkyl), S and O. In a further embodiment X is selected from CH₂, CF₂, CHF, NH, N(C₁-C₃-alkyl), S and O.

In a preferred embodiment X is selected from CH₂, NH and O. In a more preferred embodiment X is selected from NH and O. In a particular embodiment X is selected from CH₂, CO, CHOH, CHO(C₁-C₃) alkyl, NH and O. In a more particular embodiment X is selected from CH₂, CHOH, CHO(C₁-C₃) alkyl, and O, more particular X is selected from CH₂, CHOH, CHOCH₃, and O. In an even more particular preferred embodiment X is O.

In one embodiment Y¹, Y² and Y³ are each C. In a further embodiment Y¹, Y² and Y³ are each N. In a further embodiment Y¹ is C, Y² is selected from N and C and Y³ is C. In a further embodiment Y¹ and Y² are each C and Y³ is selected from N and C. In a further embodiment Y¹ and Y² are each independently selected from N and C and Y³ is C. In a further embodiment Y¹ and Y² are each independently selected from N and C and Y³ is N. In a further embodiment Y¹ and Y³ are each independently selected from N and C and Y² is C. In a further embodiment Y¹ and Y³ are each independently selected from N and C and Y² is N.

In a further embodiment Y² and Y³ are each independently selected from N and C and Y¹ is C. In a further embodiment Y² and Y³ are each independently selected from N and C and Y¹ is N. In a preferred embodiment Y¹ is N or C and Y² and Y³ are each C. In a more preferred embodiment Y¹ is N and Y² and Y³ are each C. In an even more preferred embodiment Y¹ is selected from N and C, Y² is selected from N and C and Y³ is C.

In one embodiment R¹ is selected from C₃-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl. In a further embodiment R¹ is selected from C₃-C₆ alkyl, C₂-C₆ heteroalkyl, C₂-C₆ alkenyl, C₂-C₆ alkynyl, C₃-C₁₂ cycloalkyl, and C₃-C₁₂ heterocyclyl. In a further embodiment R¹ is selected from C₃-C₆ alkyl, C₃-C₆ heteroalkyl, C₃-C₆ alkenyl, C₃-C₆ alkynyl, C₃-C₁₂ cycloalkyl, and C₃-C₁₂ heterocyclyl. In a further embodiment R¹ is selected from C₄-C₆ alkyl, C₄-C₆ heteroalkyl, C₄-C₆ alkenyl. C₄-C₆ alkynyl.

In a preferred embodiment R^{l}is selected from H, halogen, C₁-C₆ alkyl, C₁-C₆ heteroalkyl, C₃-C₁₂ cycloalkyl, C₃-C₁₂ heterocyclyl and C₁-C₆ alkoxy. In a further preferred embodiment R¹ is selected from H, methyl, ethyl, halogen, CN, CF₃, NH₂ and OH. In a more preferred embodiment R¹ is selected from H, halogen, C₁-C₆ alkyl, C₃-C₁₂ cycloalkyl and C₁-C₆ alkoxy.

In an even more preferred embodiment R¹ is selected from H, halogen and C₁-C₆ alkyl. In a particular preferred embodiment R¹ is selected from H, halogen and methyl. In a particular preferred embodiment R¹ is selected from H and C₁-C₆ alkyl, more particular R¹ is selected from H and methyl. In an even more particular preferred embodiment R¹ is H.

In one embodiment R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl.

In a further embodiment R² is selected from COC₁-C₆ alkyl, NH₂, OH, CN, SO₃H, S(O)ₙ(C₁-C₆ alkyl) wherein n is 0, 1 or 2, C₂-C₆ alkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl. In a further embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl, preferably by C₁-C₆ alkyl, C₁-C₆ heteroalkyl, halogen.

In a further embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl, preferably by C₁-C₆ alkyl, C₁-C₆ heteroalkyl, halogen. In a preferred embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In a further preferred embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In a further preferred embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl, imidazolyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In further preferred embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl, imidazolyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In a further preferred embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.In further preferred embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In a more preferred embodiment R² is selected from C₂-C₆ alkyl, C₂-C₆ heteroalkyl, C₂-C₆ alkenyl and C₂-C₆ alkynyl. In a further more preferred embodiment R² is selected from C₂-C₆ alkyl, C₂-C₆ heteroalkyl wherein the heterosubstituent is not halogen and is preferably selected from OH and NH₂, C₂-C₆ alkenyl and C₂-C₆ alkynyl. In an even more preferred embodiment R² is selected from C₂-C₆ alkyl and C₂-C₆ heteroalkyl. In an even more preferred embodiment R² is selected from C₂-C₆ alkyl and C₂-C₆ heteroalkyl wherein the heterosubstituent is not halogen and is preferably selected from OH and NH₂. In a further even preferred embodiment R² is C₂-C₆ alkyl, particularly C₃-C₆ alkyl, most particular tert-butyl.

In a further more preferred embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further more preferred embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further even more preferred embodiment R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen, preferably optionally substituted by C₁-C₆ alkyl, halogen, more preferably by methyl, halogen.

In a particular embodiment R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are selected from phenyl, pyridyl and thiazolyl, and wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl preferably optionally substituted by C₁-C₆ alkyl, halogen, more preferably by methyl, halogen.

In a further preferred embodiment R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are selected from phenyl, pyridyl and thiazolyl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further even more preferred embodiment R² is selected from C₂-C₆ alkyl, C₆ cycloalkyl, aryl and heteroaryl, wherein the aryl and the heteroaryl are selected from phenyl, pyridyl and thiazolyl, wherein the aryl and the heteroaryl are optionally substituted by C₁-C₆ alkyl, C₁-C₆ heteroalkyl, halogen, preferably optionally substituted by C₁-C₆ alkyl, halogen, more preferably by methyl, halogen.

In further embodiment R² is selected from C₃-C₆ alkyl, C₃-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl.

In further embodiment R² is selected from C₃-C₆ alkyl, C₃-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl.

In further embodiment R² is selected from C₃-C₆ alkyl, C₃-C₆ heteroalkyl, C₃-C₆ alkenyl, C₃-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl, imidazolyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In further embodiment R² is selected from C₃-C₆ alkyl, C₃-C₆ heteroalkyl wherein the heterosubstituent is not halogen and is preferably selected from OH and NH₂, C₃-C₆ alkenyl, C₃-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl, imidazolyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In further embodiment R² is selected from C₃-C₆ alkyl, C₃-C₆ heteroalkyl, C₃-C₆ alkenyl, C₃-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen.

In further embodiment R² is selected from C₃-C₆ alkyl, C₃-C₆ heteroalkyl wherein the heterosubstituent is not halogen and is preferably selected from OH and NH₂, C₃-C₆ alkenyl, C₃-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen. In a further preferred embodiment R² is selected from C₃-C₆ alkyl, C₃-C₆ heteroalkyl, C₃-C₆ alkenyl, C₃-C₆ alkynyl. In a further preferred embodiment R² is selected from C₃-C₆ alkyl, C₃-C₆ heteroalkyl wherein the heterosubstituent is not halogen and is preferably selected from OH and NH₂, C₃-C₆ alkenyl, C₃-C₆ alkynyl.

In a further preferred embodiment R² is selected from C₃-C₆ alkyl, C₃-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further preferred embodiment R² is selected from C₃-C₆ alkyl, C₃-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further even more preferred embodiment R² is selected from C₃-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen, preferably optionally substituted by C₁-C₆ alkyl, halogen, more preferably by methyl, halogen.

In a particular embodiment R² is selected from C₃-C₆ alkyl, C₃-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are selected from phenyl, pyridyl and thiazolyl, and wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, halogen, more preferably by methyl, halogen.

In a further preferred embodiment R² is selected from C₃-C₆ alkyl, C₃-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are selected from phenyl, pyridyl and thiazolyl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen.

In a further even more preferred embodiment R² is selected from C₃-C₆ alkyl, C₆ cycloalkyl, aryl and heteroaryl, wherein the aryl and the heteroaryl are selected from phenyl, pyridyl and thiazolyl, wherein the aryl and the heteroaryl are optionally substituted by C₁-C₆ alkyl, C₁-C₆ heteroalkyl, halogen, preferably optionally substituted by C₁-C₆ alkyl, halogen, more preferably by methyl, halogen.

In one embodiment R³ is selected from H, halogen, C₁-C₆ alkyl and C₁-C₆ heteroalkyl. In a preferred embodiment R³ is selected from H, halogen and C₁-C₆ alkyl. In a more preferred embodiment R³ is selected from H and halogen. In an even more preferred embodiment R³ is H.

In one embodiment R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl. In a preferred embodiment R⁴, R⁵ and R⁶ are each independently selected from H, halogen, C₁-C₆ alkoxy and C₁-C₆ alkyl.

In a more preferred mbodiment R⁴, R⁵ and R⁶ are each independently selected from H, halogen, and C₁-C₆ alkyl. In an even more preferred embodiment R⁴, R⁵ and R⁶ are each independently selected from H and halogen and are preferably H. In a particular embodiment R⁴ is selected from H and halogen, and/ or R⁵ is selected from H and C₁-C₆ alkyl and/or R⁶ is selected from H and C₁-C₆ alkyl. In a more particular embodiment R⁴ is selected from H and halogen, and/ or R⁵ is selected from H and methyl and/or R⁶ is selected from H and methyl.

In one embodiment R⁷ is absent when Y¹ is N or is selected from H, halogen, C₁-C₆ alkyl and C₁-C₆ heteroalkyl when Y¹ is C. In a preferred embodiment R⁷ is absent when Y¹ is N or is selected from H, halogen and C₁-C₆ alkyl when Y¹ is C. In a more preferred embodiment R⁷ is absent when Y¹ is N or is H when Y¹ is C.

In one embodiment R⁸ is absent when Y¹ is N or is selected from H, halogen, C₁-C₆ alkyl and C₁-C₆ heteroalkyl when Y³ is C. In a preferred embodiment R⁸ is absent when Y¹ is N or is selected from H, halogen and C₁-C₆ alkyl when Y³ is C. In a more preferred embodiment R⁸ is absent when Y¹ is N or is H when Y³ is C.

In one embodiment R⁹ is absent when Y² is N or is selected from C₃-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C¹⁻C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C. In a further embodiment R⁹ is absent when Y² is N or is selected from C₂-C₆ alkyl, C₂-C₆ heteroalkyl, C₂-C₆ alkenyl, C₂-C₆ alkynyl, C₃-C₁₂ cycloalkyl, and C₃-C₁₂ heterocyclyl when Y² is C. In a further embodiment R⁹ is absent when Y² is N or is selected from C₃-C₆ alkyl, C₃-C₆ heteroalkyl, C₃-C₆ alkenyl, C₃-C₆ alkynyl, C₃-C₁₂ cycloalkyl, and C₃-C₁₂ heterocyclyl when Y² is C. In a further embodiment R⁹ is absent when Y² is N or is selected from C₄-C₆ alkyl, C₄-C₆ heteroalkyl, C₄-C₆ alkenyl. C₄-C₆ alkynyl when Y² is C. In a preferred embodiment R⁹ is absent when Y² is N or is selected from H, halogen, C₁-C₆ alkyl, C₁-C₆ heteroalkyl, C₃-C₁₂ cycloalkyl, C₃-C₁₂ heterocyclyl and C₁-C₆ alkoxy when Y² is C. In a further preferred embodiment R⁹ is absent when Y² is N or is selected from H, methyl, ethyl, halogen, CN, CF₃, NH₂ and OH when Y² is C. In a more preferred embodiment R⁹ is absent when Y² is N or is selected from H, halogen C₁-C₆ alkyl and C₁-C₆ heteroalkyl when Y² is C. In a even more preferred embodiment R⁹ is absent when Y² is N or is selected from H, halogen and methyl when Y² is C. In a particular preferred embodiment R⁹ is absent when Y² is N or is H when Y² is C. In a more preferred embodiment Y¹ is N and R⁷ is absent, Y² and Y³ are each C and R⁹ and R⁸ are each H.

In one embodiment R¹⁹ is selected from H, C₁-C₆ alkyl. In a preferred embodiment R¹⁰ is H.

In one embodiment R¹¹ is selected from C₁-C₃ cyanoalkyl, and C₁-C₆ alkyl substituted by aryl, heteroaryl, C₃-C₁₂ heterocyclyl, preferably is C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen. In a preferred embodiment R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl and the heteroaryl are optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂.

In one embodiment R¹¹ is selected from C₁-C₃ cyanoalkyl, and C₁-C₆ alkyl substituted by heteroaryl, preferably is C₁-C₆ alkyl substituted by heteroaryl, wherein the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂.

In a further embodiment R¹¹ is selected from C₁-C₃ cyanoalkyl, and C₁-C₆ alkyl substituted by aryl, 6 to 10-membered heteroaryl, C₃-C₁₂ heterocyclyl, preferably is C₁-C₆ alkyl substituted by aryl, 6 to 10-membered heteroaryl, C₃-C₁₂ heterocyclyl, wherein the aryl, the 6 to 10-membered heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by aryl, heteroaryl, or C₃-C₁₂ heterocyclyl, wherein the heteroaryl is not 3H-imidazole-4-yl, preferably is C₁-C₆ alkyl substituted by aryl, heteroaryl, or C₃-C₁₂ heterocyclyl, wherein the heteroaryl is not 3H-imidazole-4-yl, and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by aryl, heteroaryl, or C₃-C₁₂ heterocyclyl, wherein the heteroaryl is not 3H-imidazole-4-yl and not 1H-imidazole-4-yl, preferably is C₁-C₆ alkyl substituted by aryl, heteroaryl, or C₃-C₁₂ heterocyclyl, wherein the heteroaryl is not 3H-imidazole-4-yl and not 1H-imidazole-4-yl, and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by aryl, heteroaryl, C₃-C₁₂ heterocyclyl, wherein the heteroaryl is not imidazolyl, preferably is C₁-C₆ alkyl substituted by aryl, heteroaryl, C₃-C₁₂ heterocyclyl, wherein the heteroaryl is not imidazolyl and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²-R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a preferred embodiment R¹¹ is selected from C₁-C₃ cyanoalkyl, and C₁-C₆ alkyl substituted by aryl or 6 to 10-membered heteroaryl, preferably is C₁-C₆ alkyl substituted by aryl or 6 to 10-membered heteroaryl, wherein the aryl and the 6 to 10-membered heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by aryl or heteroaryl, wherein the heteroaryl is not 3H-imidazole-4-yl, preferably is C₁-C₆ alkyl substituted by aryl or heteroaryl, wherein the heteroaryl is not 3H-imidazole-4-yl, and wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by aryl or heteroaryl, wherein the heteroaryl is not 3H-imidazole-4-yl and not 1H-imidazole-4-yl, preferably is C₁-C₆ alkyl substituted by aryl or heteroaryl, wherein the heteroaryl is not 3H-imidazole-4-yl and not 1H-imidazole-4-yl, and wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by aryl or heteroaryl, wherein the heteroaryl is not imidazolyl, preferably is C₁-C₆ alkyl substituted by aryl or heteroaryl, wherein the heteroaryl is not imidazolyl and wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In one embodiment R¹¹ is selected from C₁-C₃ cyanoalkyl, and C₁-C₆ alkyl substituted by 6 to 10-membered heteroaryl, preferably is C₁-C₆ alkyl substituted by 6 to 10-membered heteroaryl, wherein the 6 to 10-membered heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl , more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by heteroaryl, wherein the heteroaryl is not 3H-imidazole-4-yl, preferably is C₁-C₆ alkyl substituted by heteroaryl, wherein the heteroaryl is not 3H-imidazole-4-yl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by heteroaryl, wherein the heteroaryl is not 3H-imidazole-4-yl and not 1H-imidazole-4-yl, preferably is C₁-C₆ alkyl substituted by heteroaryl, wherein the heteroaryl is not 3H-imidazole-4-yl and not 1H-imidazole-4-yl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further embodiment R¹¹ is selected from C₁-C₃ cyanoalkyl, and C₁-C₆ alkyl substituted by heteroaryl, wherein the heteroaryl is not imidazolyl, preferably is C₁-C₆ alkyl substituted by heteroaryl, wherein the heteroaryl is not imidazolyl and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a preferred embodiment R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the heteroaryl is selected from pyridyl, pyridazinyl, pyrimidinyl, pyrazinyl, pyrazolyl, triazolyl, tetrazolyl, thiazolyl, imidazolyl, oxazolyl, oxadiazolyl, thiophenyl, furanyl and thiadiazolyl, preferably selected from from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and the aryl is phenyl, wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the heteroaryl is selected from pyridyl, pyrimidinyl, oxadiazolyl, imidazolyl and tetrazolyl, preferably selected from pyridyl, pyrimidinyl, oxadiazolyl, and tetrazolyl, and the aryl is phenyl, wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, pyridazinyl, pyrimidinyl, pyrazinyl, pyrazolyl, triazolyl, tetrazolyl, thiazolyl, imidazolyl, oxazolyl, oxadiazolyl, thiophenyl, furanyl and thiadiazolyl, preferably selected from from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and

wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, pyrimidinyl, oxadiazolyl, imidazolyl and tetrazolyl, preferably selected from pyridyl, pyrimidinyl, oxadiazolyl, and tetrazolyl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further preferred embodiment R¹¹ is selected from C₁-C₃ cyanoalkyl, and C₁-C₆ alkyl substituted by aryl or 6 to 10-membered heteroaryl, preferably is C₁-C₆ alkyl substituted by aryl or 6 to 10-membered heteroaryl, wherein the heteroaryl is selected from pyridyl, pyridazinyl, and pyrimidinyl, preferably is selected from pyridyl and pyrimidinyl, and the aryl is phenyl , and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a more preferred embodiment R¹¹ is selected from C₁-C₃ cyanoalkyl, and C₁-C₆ alkyl substituted by aryl or heteroaryl, wherein the heteroaryl is not 3H-imidazole-4-yl, preferably is C₁-C₆ alkyl substituted by aryl or heteroaryl wherein the heteroaryl is not 3H-imidazole-4-yl, wherein the heteroaryl is selected from pyridyl, pyridazinyl, pyrimidinyl, pyrazinyl, pyrazolyl, triazolyl, tetrazolyl, thiazolyl, imidazolyl, oxazolyl, oxadiazolyl, thiophenyl, furanyl and thiadiazolyl, preferably selected from from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and the aryl is phenyl , and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a more preferred embodiment R¹¹ is selected from C₁-C₃ cyanoalkyl, and C₁-C₆ alkyl substituted by aryl or heteroaryl, wherein the heteroaryl is not 3H-imidazole-4-yl, preferably is C₁-C₆ alkyl substituted by aryl or heteroaryl wherein the heteroaryl is not 3H-imidazole-4-yl, wherein the heteroaryl is selected from pyridyl, pyrimidinyl, oxadiazolyl, imidazolyl and tetrazolyl, preferably selected from pyridyl, pyrimidinyl, oxadiazolyl, and tetrazolyl, and the aryl is phenyl , and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further more preferred embodiment R¹¹ is selected from C₁-C₃ cyanoalkyl, and C₁-C₆ alkyl substituted by aryl or heteroaryl, wherein the heteroaryl is not 3H-imidazole-4-yl and not 1H-imidazole-4-yl, preferably is C₁-C₆ alkyl substituted by aryl or heteroaryl wherein the heteroaryl is not 3H-imidazole-4-yl and not 1H-imidazole-4-yl, wherein the heteroaryl is selected from pyridyl, pyridazinyl, pyrimidinyl, pyrazinyl, pyrazolyl, triazolyl, tetrazolyl, thiazolyl, imidazolyl, oxazolyl, oxadiazolyl, thiophenyl, furanyl and thiadiazolyl, preferably selected from from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and the aryl is phenyl, and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further more preferred embodiment R¹¹ is selected from C₁-C₃ cyanoalkyl, and C₁-C₆ alkyl substituted by aryl or heteroaryl, wherein the heteroaryl is not 3H-imidazole-4-yl and not 1H-imidazole-4-yl, preferably is C₁-C₆ alkyl substituted by aryl or heteroaryl wherein the heteroaryl is not 3H-imidazole-4-yl and not 1H-imidazole-4-yl, wherein the heteroaryl is selected from pyridyl, pyrimidinyl, oxadiazolyl, imidazolyl and tetrazolyl, preferably selected from pyridyl, pyrimidinyl, oxadiazolyl, and tetrazolyl, and the aryl is phenyl, and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In a further more preferred embodiment R¹¹ is selected from C₁-C₃ cyanoalkyl, and C₁-C₆ alkyl substituted by aryl or heteroaryl, wherein the heteroaryl is selected from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl and the aryl is phenyl , preferably is C₁-C₆ alkyl substituted by aryl or heteroaryl wherein the heteroaryl is selected from pyridyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and the aryl is phenyl, and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

In an even more preferred embodiment R¹¹ is selected from C₁-C₃ cyanoalkyl, and C₁-C₆ alkyl substituted by aryl or heteroaryl, wherein the heteroaryl is selected from pyridyl, pyrimidinyl, oxadiazolyl, and tetrazolyl and the aryl is phenyl , preferably is C₁-C₆ alkyl substituted by aryl or heteroaryl wherein the heteroaryl is selected from pyridyl, pyrimidinyl, oxadiazolyl, and tetrazolyl, and the aryl is phenyl, and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²-R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂.

The aryl, the heteroaryl and the C₃-C₁₂ heterocyclyl of the substituted C₁-C₆ alkyl group of R¹⁰ and/or R¹¹ are optionally substituted usually in ortho or para position, preferably para position, or in 2 or 3 position, to the C₁-C₆ alkyl group.

In one embodiment R¹² and R¹³ are each independently selected from H, C₁-C₆ alkyl, C₁-C₆ heteroalkyl, C₂-C₆ alkenyl and C₂-C₆ alkynyl. In further embodiment R¹² and R¹³ are each independently selected from C₃-C₁₂ cycloalkyl and C₃-C₁₂ heterocyclyl. In a preferred embodiment R¹² and R¹³ are each independently selected from H, C₁-C₆ alkyl and C₁-C₆ heteroalkyl. In a more preferred embodiment R¹² and R¹³ are each independently selected from H and C₁-C₆ alkyl and are preferably H.

In a preferred embodiment

R¹ is selected from H, methyl, ethyl, halogen, CN, CF₃, NH₂ and OH and is preferably selected from H, methyl, ethyl and halogen, more preferably from H, methyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl; and

R⁹ is absent when Y² is N or is selected from H, methyl, ethyl, halogen, CN, CF₃, NH₂ and OH, preferably selected from H, methyl, ethyl and halogen when Y² is C.

In a further preferred embodiment

R¹ is selected from H, methyl, ethyl, halogen, CN, CF₃, NH₂ and OH and is preferably selected from H, methyl, ethyl and halogen, more preferably from H, methyl;

R² is selected from C₂-C₆ alkyl, C₂-C₆ heteroalkyl wherein the C₂-C₆ heteroalkyl is preferably C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl; and

R⁹ is absent when Y² is N or is selected from H, methyl, ethyl, halogen, CN, CF₃, NH₂ and OH, preferably selected from H, methyl, ethyl and halogen when Y² is C .

In a further preferred embodiment

R¹ is selected from H, methyl, ethyl, halogen, CN, CF₃, NH₂ and OH and is preferably selected from H, methyl, ethyl and halogen, more preferably from H, methyl;

R² is selected from C₂-C₆ alkyl, C₂-C₆ heteroalkyl wherein the C₂-C₆ heteroalkyl is preferably C₂-C₆ heteroalkyl wherein the heterosubstituent is not halogen and is preferably selected from OH and NH₂, C₂-C₆ alkenyl and C₂-C₆ alkynyl; and

R⁹ is absent when Y² is N or is selected from H, methyl, ethyl, halogen, CN, CF₃, NH₂ and OH preferably selected from H, methyl, ethyl and halogen when Y² is C .

In a further preferred embodiment

R¹ is selected from H, methyl, ethyl, halogen, CN, CF₃, NH₂ and OH and is preferably selected from H, methyl, ethyl and halogen, more preferably from H, methyl;

R² is selected from C₂-C₆ alkyl and C₂-C₆ heteroalkyl wherein the C₂-C₆ heteroalkyl is preferably C₂-C₆ heteroalkyl wherein the heterosubstituent is not halogen and is preferably selected from OH and NH₂, and is preferably tert-butyl; and

R⁹ is absent when Y² is N or is selected from H, methyl, ethyl, halogen, CN, CF₃, NH₂ and OH, preferably selected from H, methyl, ethyl and halogen when Y² is C .

In a further preferred embodiment

R¹ is selected from H, methyl, ethyl, halogen, CN, CF₃, NH₂ and OH and is preferably selected from H, methyl, ethyl and halogen, more preferably from H, methyl;

R² is selected from C₃-C₆ alkyl, C₃-C₆ heteroalkyl wherein the C₃-C₆ heteroalkyl is preferably C₃-C₆ heteroalkyl wherein the heterosubstituent is not halogen and is preferably selected from OH and NH₂, C₃-C₆ alkenyl, C₃-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl; and

R⁹ is absent when Y² is N or is selected from H, methyl, ethyl, halogen, CN, CF₃, NH₂ and OH, preferably selected from H, methyl, ethyl and halogen when Y² is C .

In a further preferred embodiment

R¹ is selected from H, methyl, ethyl, halogen, CN, CF₃, NH₂ and OH and is preferably selected from H, methyl, ethyl and halogen and is more preferably H, methyl;

R² is selected from C₃-C₆ alkyl, C₃-C₆ heteroalkyl wherein the C₃-C₆ heteroalkyl is preferably C₃-C₆ heteroalkyl wherein the heterosubstituent is not halogen and is preferably selected from OH and NH₂, C₃-C₆ alkenyl, C₃-C₆ alkynyl, C₃-C₁₂ cycloalkyl, C₃-C₁₂ heterocyclyl, aryl and heteroaryl, wherein the aryl and the heteroaryl are preferably selected from phenyl, pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl; and

R⁹ is absent when Y² is N or is selected from H, methyl, ethyl, halogen, CN, CF₃, NH₂ and OH, preferably selected from H, methyl, ethyl and halogen and is more preferably H when Y² is C .

In a further preferred embodiment

R¹ is selected from H, methyl, ethyl, halogen, CN, CF₃, NH₂ and OH and is preferably selected from H, methyl, ethyl and halogen and is more preferably H, methyl;

R² is selected from C₃-C₆ alkyl, C₃-C₆ heteroalkyl wherein the C₃-C₆ heteroalkyl is preferably C₃-C₆ heteroalkyl wherein the heterosubstituent is not halogen and is preferably selected from OH and NH₂, C₃-C₆ alkenyl and C₃-C₆ alkynyl; and

R⁹ is absent when Y² is N or is selected from H, methyl, ethyl, halogen, CN, CF₃, NH₂ and OH, preferably selected from H, methyl, ethyl and halogen and is more preferably H when Y² is C .

In a further preferred embodiment

R¹ is selected from H, methyl, ethyl, halogen, CN, CF₃, NH₂ and OH and is preferably selected from H, methyl, ethyl and halogen and is more preferably H, methyl;

R² is selected from C₃-C₆ alkyl and C₃-C₆ heteroalkyl wherein the C₃-C₆ heteroalkyl is preferably C₃-C₆ heteroalkyl wherein the heterosubstituent is not halogen and is preferably selected from OH and NH₂ wherein R² is is preferably C₃-C₆ alkyl more preferably tert-butyl; and

R⁹ is absent when Y² is N or is selected from H, methyl, ethyl, halogen, CN, CF₃, NH₂ and OH preferably selected from H, methyl, ethyl and halogen and is more preferably H when Y² is C .

In a further preferred embodiment

R¹ is selected from H, methyl and ethyl and is more preferably H, methyl;

R² is selected from C₃-C₆ alkyl and C₃-C₆ heteroalkyl wherein the C₃-C₆ heteroalkyl is preferably C₃-C₆ heteroalkyl wherein the heterosubstituent is not halogen and is preferably selected from OH and NH₂; and

R⁹ is absent when Y² is N or is selected from H, methyl and ethyl when Y² is C.

In a more preferred embodiment

R¹ is selected from H, methyl and ethyl, and is preferably H;

R² is C₃-C₆ alkyl, preferably t-butyl; and

R⁹ is absent when Y² is N or is selected from H, methyl and ethyl and is preferably H when Y² is C.

In a particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C ₁ -C₆ heteroalkyl, halogen, CN, C₃-C₁₂ cycloalkyl, C₃-C₁₂ heterocyclyl, C(O)NR¹², C(O)NR¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl, more preferably is C₁-C₆ alkyl substituted by aryl or heteroaryl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is preferably selected from COC₁-C₆ alkyl, NH₂, OH, CN, SO₃H, S(O)ₙ(C₁-C₆ alkyl) wherein n is 0, 1 or 2, C₂-C₆ alkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from

N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl, preferably is C₁-C₆ alkyl substituted by aryl or heteroaryl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from C₂-C₆ alkyl, C₂-C₆ heteroalkyl wherein the C₂-C₆ heteroalkyl is preferably C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, aryl and heteroaryl, wherein the aryl and the heteroaryl are optionally substituted by C₁-C₆ alkyl, C₁-C₆ heteroalkyl, CN, halogen, C₃-C₁₂ cycloalkyl ,C₃-C₁₂ heterocyclyl, preferably by C₁-C₆ alkyl, C₁-C₆ heteroalkyl, halogen;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C¹-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl, preferably is C₁-C₆ alkyl substituted by aryl or heteroaryl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl and C₂-C₆ alkynyl; R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocycly, preferably selected from H, and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; Z is NR¹⁰R¹¹, wherein

R¹⁰ is H; and

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl, preferably is C₁-C₆ alkyl substituted by aryl or heteroaryl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl and C₂-C₆ alkynyl;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C¹-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocycly, preferably selected from H, and C₁-C₆ alkyl.

In a particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from

N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, 6 to 10-membered heteroaryl, or C₃-C₁₂ heterocyclyl, wherein the aryl, the 6 to 10-membered heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³ , preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by c 6 to 10-membered heteroaryl, preferably is C₁-C₆ alkyl substituted by aryl or 6 to 10-membered heteroaryl, wherein the aryl and the 6 to 10-membered heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl,

wherein R² is more preferably selected from C₂-C₆ alkyl, C₂-C₆ heteroalkyl, C₂-C₆ alkenyl and C₂-C₆ alkynyl;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from

N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, heteroaryl, or C₃-C₁₂ heterocyclyl, wherein the heteroaryl is not imidazolyl and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl wherein the heteroaryl is not imidazolyl, preferably is C₁-C₆ alkyl substituted by aryl or heteroaryl wherein the heteroaryl is not imidazolyl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl;

R³ is selected from H, halogen, C₁-C₆ alkyl and C₁-C₆ heteroalkyl, preferably selected from H, halogen and C₁-C₆ alkyl; R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; Z is NR¹⁰R¹¹, wherein

R¹⁰ is H; and

R¹¹ is selected from C₁-C₃ cyanoalkyl, and C₁-C₆ alkyl substituted by aryl, heteroaryl, or C₃-C₁₂ heterocyclyl, wherein the heteroaryl is not 3H-imidazole-4-yl and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl wherein the heteroaryl is not 3H-imidazole-4-yl, preferably is C₁-C₆ alkyl substituted by aryl or heteroaryl wherein the heteroaryl is not 3H-imidazole-4-yl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂,

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl,

wherein R² is more preferably selected from C₂-C₆ alkyl, C₂-C₆ heteroalkyl, C₂-C₆ alkenyl and C₂-C₆ alkynyl;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a further particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from

N and C, Y² is selected from N and C and Y³ is C;

Z is NR¹⁰R¹¹, wherein

R¹⁰ is H; and

R¹¹ is selected from C₁-C₃ cyanoalkyl, and C₁-C₆ alkyl substituted by aryl, heteroaryl, or C₃-C₁₂ heterocyclyl, wherein the heteroaryl is not 3H-imidazole-4-yl and not 1H-imidazole-4-yl and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, wherein R¹¹ is preferably selected from C₁-C₃ cyanoalkyl and C₁-C₆ alkyl substituted by aryl or heteroaryl wherein the heteroaryl is not 3H-imidazole-4-yl and not 1H-imidazole-4-yl, preferably is C₁-C₆ alkyl substituted by aryl or heteroaryl wherein the heteroaryl is not 3H-imidazole-4-yl and not 1H-imidazole-4-yl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl, preferably by NH₂, C₁-C₆ alkyl, C₁-C₆ heteroalkyl, halogen, more preferably by NH₂;

R¹ is selected from H, halogen, C₁-C₆ alkyl, C₃-C₁₂ cycloalkyl and C₁-C₆ alkoxy, preferably selected from H, halogen and C₁-C₆ alkyl;

R² is selected from COC₁-C₆ alkyl, NH₂, OH, CN, SO₃H, S(O)ₙ(C₁-C₆ alkyl) wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably by C₁-C₆ alkyl, C₁-C₆ heteroalkyl, halogen, wherein R² is preferably selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl,

wherein R² is more preferably selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen, C₁-C₆ alkyl and C₁-C₆ heteroalkyl, preferably selected from H, halogen and C₁-C₆ alkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl, preferably selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl, preferably selected from H and C₁-C₆ alkyl.

In a more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from

N and C, Y² is selected from N and C and Y³ is C; R¹⁰ is H;

R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl is phenyl and

wherein the heteroaryl is selected from pyridinyl, pyridazinyl, pyrimidinyl, pyrazinyl, pyrazolyl, triazolyl, tetrazolyl, thiazolyl, oxazolyl, oxadiazolyl, imidazolyl, thiophenyl, furanyl and thiadiazolyl, preferably selected from pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl and wherein the aryl and the heteroaryl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and R¹² and R¹¹ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridinyl, pyridazinyl, pyrimidinyl, pyrazinyl, pyrazolyl, triazolyl, tetrazolyl, thiazolyl, oxazolyl, oxadiazolyl, imidazolyl, thiophenyl, furanyl and thiadiazolyl, preferably selected from pyridinyl, pyridazinyl, pyrimidinyl, pyrazinyl, pyrazolyl, triazolyl, tetrazolyl, thiazolyl, oxazolyl, oxadiazolyl, thiophenyl, furanyl and thiadiazolyl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C¹-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

R¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl is phenyl and wherein the heteroaryl is selected from pyridinyl, pyridazinyl, pyrimidinyl, pyrazinyl, pyrazolyl, triazolyl, tetrazolyl, thiazolyl, oxazolyl, oxadiazolyl, imidazolyl, thiophenyl, furanyl and thiadiazolyl and wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂,

R¹ is selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

R¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridinyl, pyridazinyl, pyrimidinyl, pyrazinyl, pyrazolyl, triazolyl, tetrazolyl, thiazolyl, oxazolyl, oxadiazolyl, imidazolyl, thiophenyl, furanyl and thiadiazolyl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

R¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, pyrimidinyl, imidazolyl, oxadiazolyl and tetrazolyl, preferably from pyridyl, pyrimidinyl, oxadiazolyl and tetrazolyl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and

R¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C;

R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, pyrimidinyl, imidazolyl, oxadiazolyl and tetrazolyl, preferably from pyridyl, pyrimidinyl, oxadiazolyl and tetrazolyl, and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; and R¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H; R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, preferably selected from pyridyl, pyrimidinyl, oxadiazolyl and tetrazolyl, wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C¹-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is

N or is selected from H, halogen and C₁-C₆ alkyl when Y² is C; andR¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, preferably selected from pyridyl, pyrimidinyl, oxadiazolyl and tetrazolyl, wherein the aryl and the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;

R¹ is selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; andR¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, pyrimidinyl, imidazolyl, oxadiazolyl and tetrazolyl, preferably selected from pyridyl, pyrimidinyl, oxadiazolyl and tetrazolyl, wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;,

R¹ is selected from H, halogen and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; andR¹² and R¹³ are each independently selected from H and C₁-C₆ alkyl.

In a further more particular preferred embodiment

X is selected from CH₂, CO, CHOH, CHO(C₁-C₃) alkyl, NH and O and is preferably selected from CH₂, CHOH, CHO(C₁-C₃) alkyl, and O;

Y¹, Y², and Y³ are each independently selected from N and C, preferably Y¹ is selected from N and C, Y² is selected from N and C and Y³ is C; R¹⁰ is H;

R¹¹ is selected from C₁-C₃ cyanoalkyl, and C₁-C₆ alkyl substituted by heteroaryl, preferably is C₁-C₆ alkyl substituted by heteroaryl, wherein the heteroaryl is selected from pyridyl, imidazolyl, oxadiazolyl and tetrazolyl and wherein the heteroaryl is optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³, preferably by NH₂, C₁-C₆ alkyl, C₁-C₆ heteroalkyl, halogen, CN, C₃-C₁₂ cycloalkyl, C₃-C₁₂ heterocyclyl, more preferably by NH₂, C₁-C₆ alkyl, C₁-C₆ heteroalkyl, halogen, even more preferably by NH₂;,

R¹ is selected from H, halogen and C₁-C₆ alkyl;

R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, wherein R² is more preferably selected from C₂-C₆ alkyl, C₂-C₆ heteroalkyl, C₂-C₆ alkenyl and C₂-C₆ alkynyl, even more preferably selected from C₂-C₆ alkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen, C₁-C₆ alkyl and, C₁-C₆ heteroalkyl, preferably selected from H, halogen and C₁-C₆ alkyl when Y¹ is C, or R⁷ is absent when Y¹ is N or is selected from H, halogen and C^{l}-C⁶ alkyl when Y¹ is C;

R⁸ is absent when Y³ is N or is selected from H, halogen, C₁-C₆ alkyl and, C₁-C₆ heteroalkyl, preferably selected from H, halogen and C₁-C₆ alkyl when Y³ is C;

R⁹ is absent when Y² is N or is selected from H, halogen, C₁-C₆ alkyl and, C₁-C₆ heteroalkyl, preferably selected from H, halogen and C₁-C₆ alkyl when Y² is C, or R⁹ is absent when Y² is N or is selected from H, halogen and C₁-C₆ alkyl when Y² is C; andR¹² and R¹³ are each independently selected from H and C₁-C₆ alkyl.

In an even more particular preferred embodiment

X is selected from CH₂, CF₂, CHF, CO, CHOH, CHO(C₁-C₃) alkyl, NH, N(C₁-C₃ alkyl), S, SO and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl, preferably optionally substituted by NH₂, C₁-C₆ alkyl, C₁-C₆ heteroalkyl, halogen;

R¹ is selected from H, halogen, C₁-C₆ alkyl, C₃-C₁₂ cycloalkyl and C₁-C₆ alkoxy;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen;

R³ is selected from H, halogen, C₁-C₆ alkyl and C₁-C₆ heteroalkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C;

R⁹ is absent when Y² is N or is selected from H, halogen and C₁-C₆ alkyl when Y² is C.

In a further even more particular preferred embodiment

X is selected from CH₂, CF₂, CHF, CO, CHOH, CHO(C₁-C₃) alkyl, NH, N(C₁-C₃ alkyl), S, SO and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl, preferably optionally substituted by NH₂, C₁-C₆ alkyl, C₁-C₆ heteroalkyl, halogen;

R¹ is selected from H, halogen, and C₁-C₆ alkyl;

R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl, preferably optionally substituted by C₁-C₆ alkyl, C₁-C₆ heteroalkyl, halogen, more preferably by C₁-C₆ alkyl, halogen, even more preferably by methyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C;

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O, preferably X is selected from CH₂, CHOH, CHO(C₁-C₃) alkyl, and O;

Y¹, Y², and Y³ are each independently selected from N and C;

Z is NR¹⁰R¹¹;

R¹⁰ H;

R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;

R¹¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O, preferably X is selected from CH₂, CHOH, CHO(C₁-C₃) alkyl, and O;

Y¹ is selected from N and C, Y² is selected from N and C and Y³ is C;

Z is NR¹⁰R¹¹;

R¹⁰ is H;

R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is selected from H, halogen and C₁-C₆ alkyl; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O, preferably X is selected from CH₂, CHOH, CHO(C₁-C₃) alkyl, and O;

wherein Y¹, Y², and Y³ are each independently selected from N and C;

Z is NR¹⁰R¹¹;

R¹⁹ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O, preferably X is selected from CH₂, CHOH, CHO(C₁-C₃) alkyl, and O;

Y¹ is selected from N and C, Y² is selected from N and C and Y³ is C;

Z is NR¹⁰R¹¹;

R¹⁰ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is selected from H, halogen and C₁-C₆ alkyl; and

R⁹ is absent when Y² is N or is selected from H, halogen and C₁-C₆ alkyl when Y² is C.

With respect to the even more particular preferred embodiments outlined above, the heteroaryl group of R¹¹ is preferably not 3H-imidazole-4-yl.

With respect to the even more particular preferred embodiments outlined above, the heteroaryl group of R¹¹ is preferably not 3H-imidazole-4-yl and not 1H-imidazole-4-yl.

In a further even more particular preferred embodiment

X is selected from CH₂, CO, CHOH, CHO(C₁-C₃) alkyl, NH and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O, preferably X is selected from CH₂, CHOH, CHO(C₁-C₃) alkyl, and O;

Y¹ is selected from N and C, Y² is selected from N and C and Y³ is C;

Z is NR¹⁰R¹¹;

R¹⁰ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is selected from H, halogen and C₁-C₆ alkyl; and

R⁹ is absent when Y² is N or is selected from H, halogen and C₁-C₆ alkyl when Y² is C.

In a further even more particular preferred embodiment

X is selected from CH₂, CF₂, CHF, CO, CHOH, CHO(C₁-C₃) alkyl, NH, N(C₁-C₃ alkyl), S, SO and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;

R¹ is selected from H, halogen, C₁-C₆ alkyl, C₃-C₁₂ cycloalkyl and C₁-C₆ alkoxy;

R² is selected from C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

R³ is selected from H, halogen, C₁-C₆ alkyl and C₁-C₆ heteroalkyl;

R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C;

R⁹ is absent when Y² is N or is selected from H, halogen and C₁-C₆ alkyl when Y² is C.

In a further even more particular preferred embodiment

X is selected from CH₂, CF₂, CHF, CO, CHOH, CHO(C₁-C₃) alkyl, NH, N(C₁-C₃ alkyl), S, SO and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;

R¹ is selected from H, halogen, and C₁-C₆ alkyl;

R² is selected from C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C;

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O;

Y¹, Y², and Y³ are each independently selected from N and C;

Z is NR¹⁰R¹¹;

R¹⁰ is H;

R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O;

Y¹ is selected from N and C, Y² is selected from N and C and Y³ is C;

Z is NR¹⁹R¹¹;

R¹⁰ is H;

R¹¹ is selected from C₁-C₃ cyanoalkyl and C₁-C₆ alkyl substituted by aryl or heteroaryl,

wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is selected from H, halogen and C₁-C₆ alkyl; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O;

wherein Y¹, Y², and Y³ are each independently selected from N and C;

Z is NR¹⁰R¹¹;

R¹⁰ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R⁸ is absent when Y³ is N or is selected from H, halogen and C₁-C₆ alkyl when Y³ is C; and

R⁹ is absent when Y² is N or is selected from H, halogen and C₁-C₆ alkyl when Y² is C.

In a further even more particular preferred embodiment

X is selected from CH₂, CO, CHOH, CHO(C₁-C₃) alkyl, NH and O;

Y¹ is selected from N and C, Y² is selected from N and C and Y³ is C;

Z is NR¹⁰R¹¹;

R¹⁰ is H;

R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;

R¹ is selected from H, halogen and C₁-C₆ alkyl;

R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;

R³ is selected from H, halogen and C₁-C₆ alkyl;

R⁴, R⁵ and R⁶ are each independently selected from H, halogen and C₁-C₆ alkyl;

R⁷ is absent when Y¹ is N or is selected from H, halogen and C₁-C₆ alkyl when Y¹ is C;

R^{g} is selected from H, halogen and C₁-C₆ alkyl; and

R⁹ is absent when Y² is N or is selected from H, halogen and C₁-C₆ alkyl when Y² is C.

A most particular preferred embodiment of the invention are the following compounds pharmaceutically-accentahle salts, hydrates, solvates, or stereoisomers thereof:

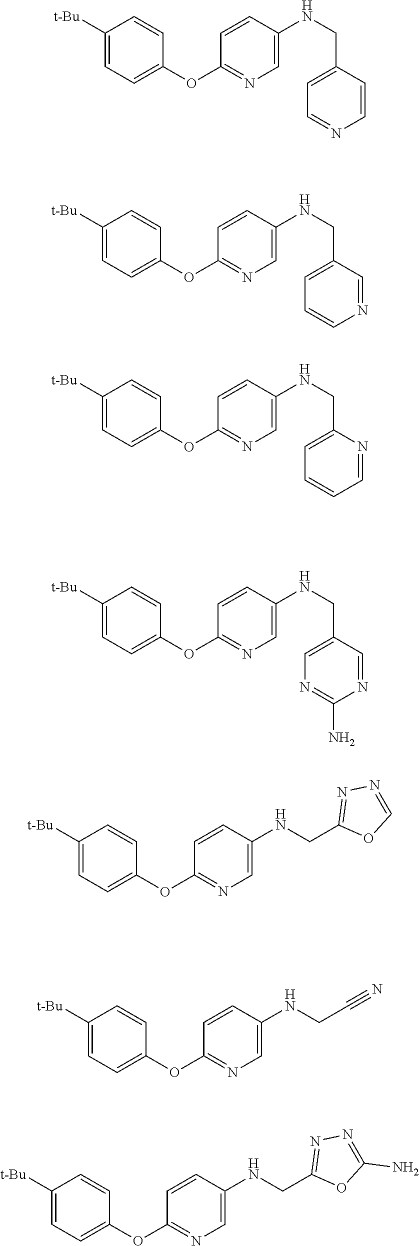

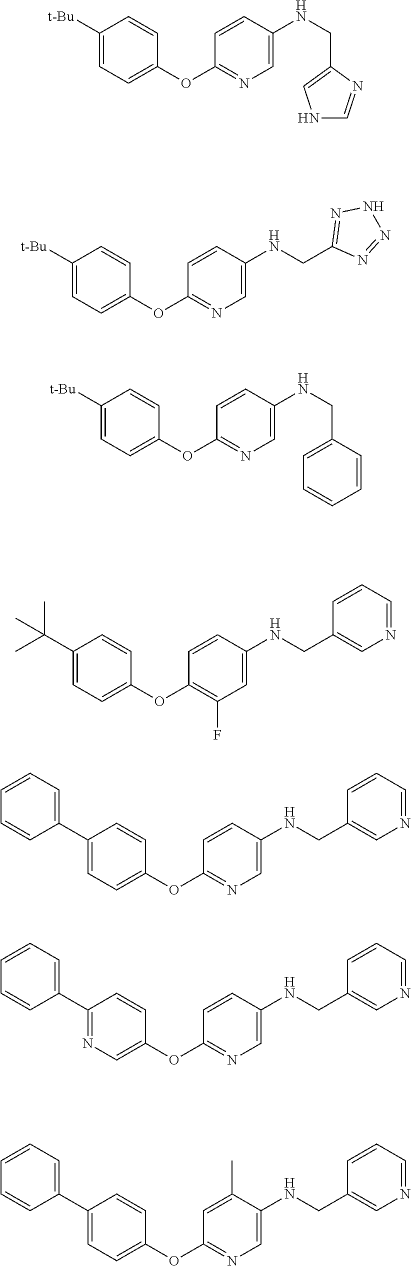

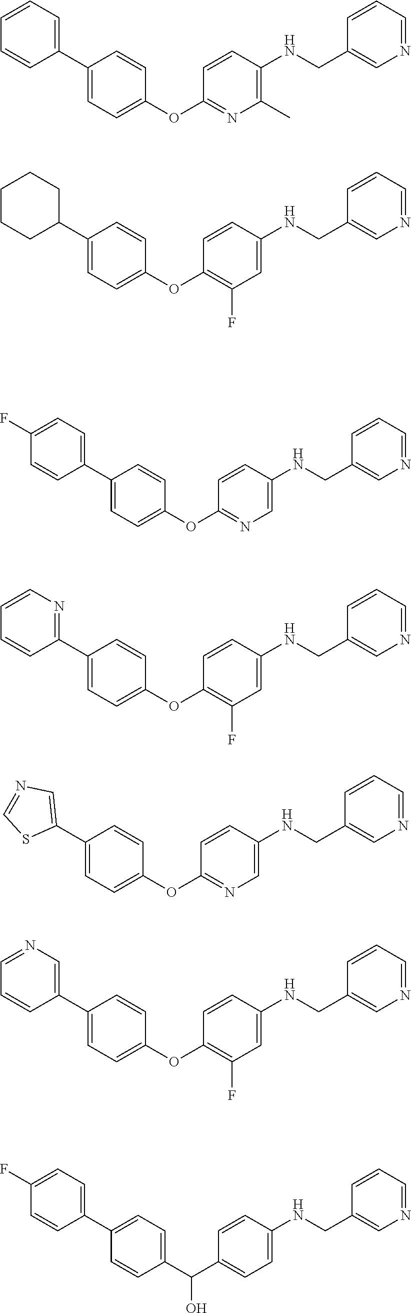

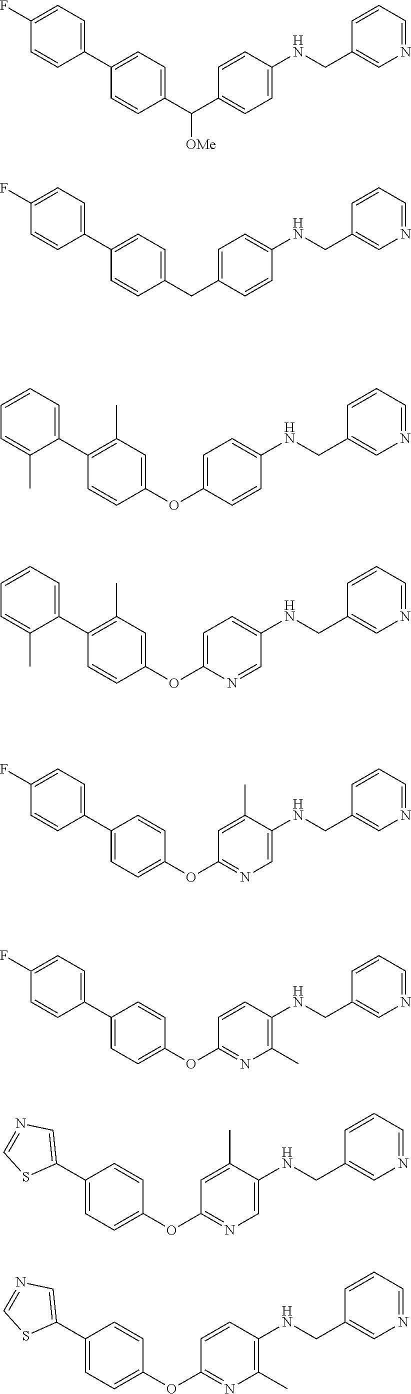

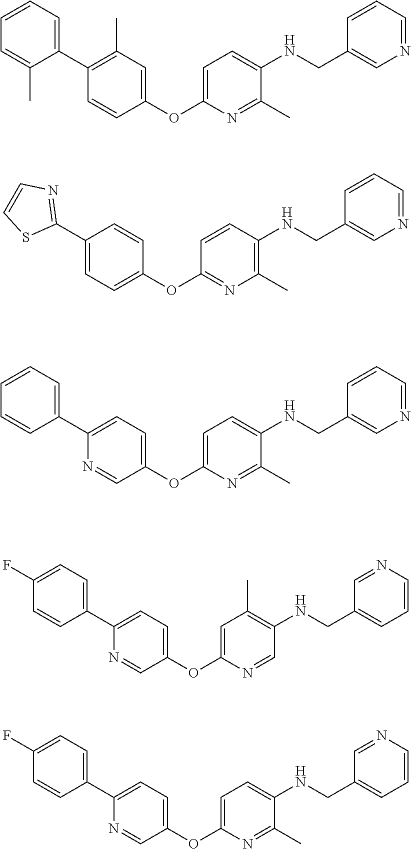 [SMILES: Cc1ccccc1-c1ccc(Oc2ccc(NCc3cccnc3)c(C)n2)cc1C ; Cc1nc(Oc2ccc(-c3nccs3)cc2)ccc1NCc1cccnc1 ; Cc1nc(Oc2ccc(-c3ccccc3)nc2)ccc1NCc1cccnc1 ; Cc1cc(Oc2ccc(-c3ccc(F)cc3)nc2)ncc1NCc1cccnc1 ; Cc1nc(Oc2ccc(-c3ccc(F)cc3)nc2)ccc1NCc1cccnc1]

A further most particular preferred embodiment of the invention are the following compounds pharmaceutically-acceptable salts, hydrates, solvates, or stereoisomers thereof:

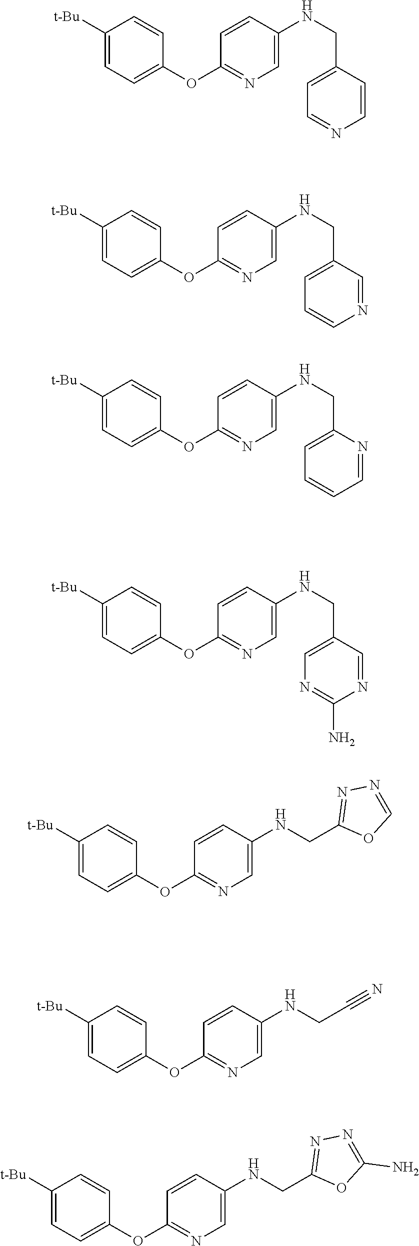

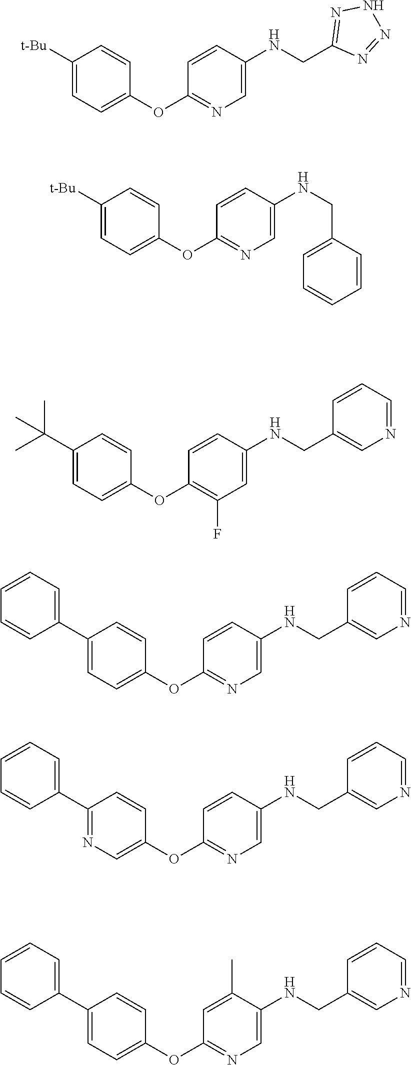 [SMILES: CC(C)(C)c1ccc(Oc2ccc(NCc3nn[nH]n3)cn2)cc1 ; CC(C)(C)c1ccc(Oc2ccc(NCc3ccccc3)cn2)cc1 ; CC(C)(C)c1ccc(Oc2ccc(NCc3cccnc3)cc2F)cc1 ; c1ccc(-c2ccc(Oc3ccc(NCc4cccnc4)cn3)cc2)cc1 ; c1ccc(-c2ccc(Oc3ccc(NCc4cccnc4)cn3)cn2)cc1 ; Cc1cc(Oc2ccc(-c3ccccc3)cc2)ncc1NCc1cccnc1]

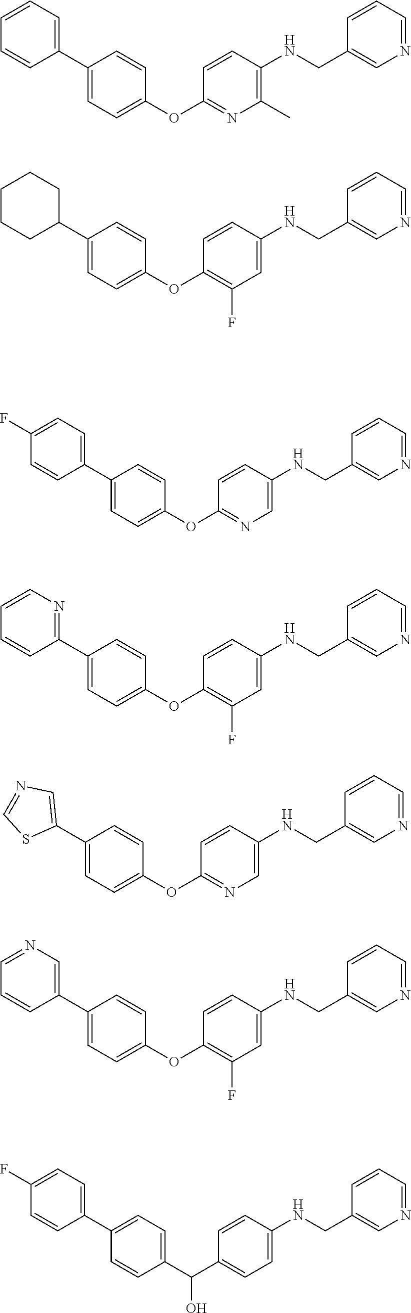

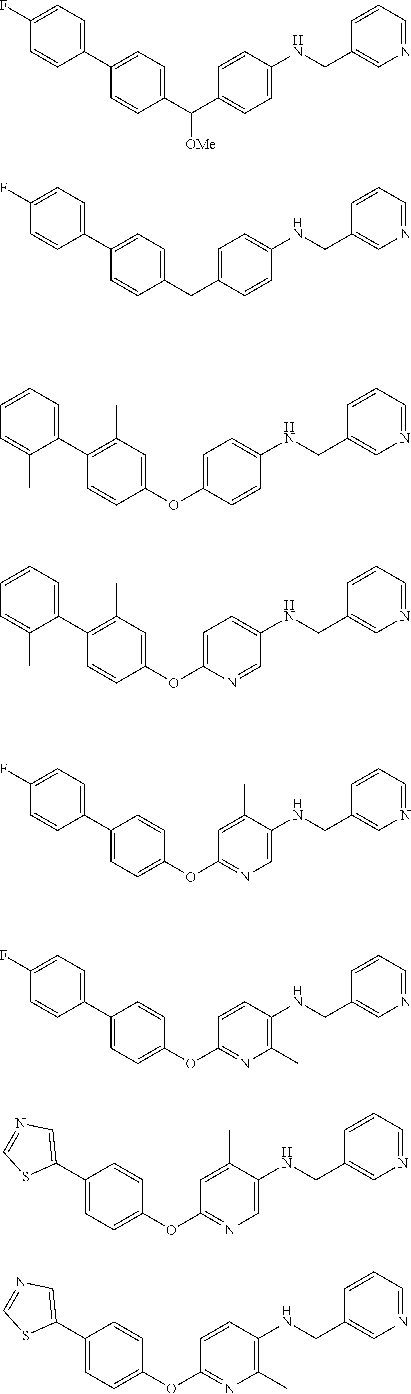

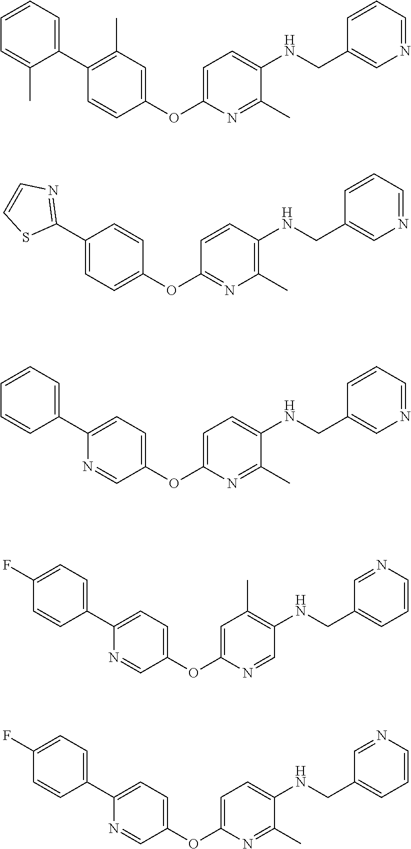 [SMILES: Cc1ccccc1-c1ccc(Oc2ccc(NCc3cccnc3)c(C)n2)cc1C ; Cc1nc(Oc2ccc(-c3nccs3)cc2)ccc1NCc1cccnc1 ; Cc1nc(Oc2ccc(-c3ccccc3)nc2)ccc1NCc1cccnc1 ; Cc1cc(Oc2ccc(-c3ccc(F)cc3)nc2)ncc1NCc1cccnc1 ; Cc1nc(Oc2ccc(-c3ccc(F)cc3)nc2)ccc1NCc1cccnc1]

A further most particular preferred embodiment of the invention are the following compounds pharmaceutically-acceptable salts, hydrates, solvates, or stereoisomers thereof:

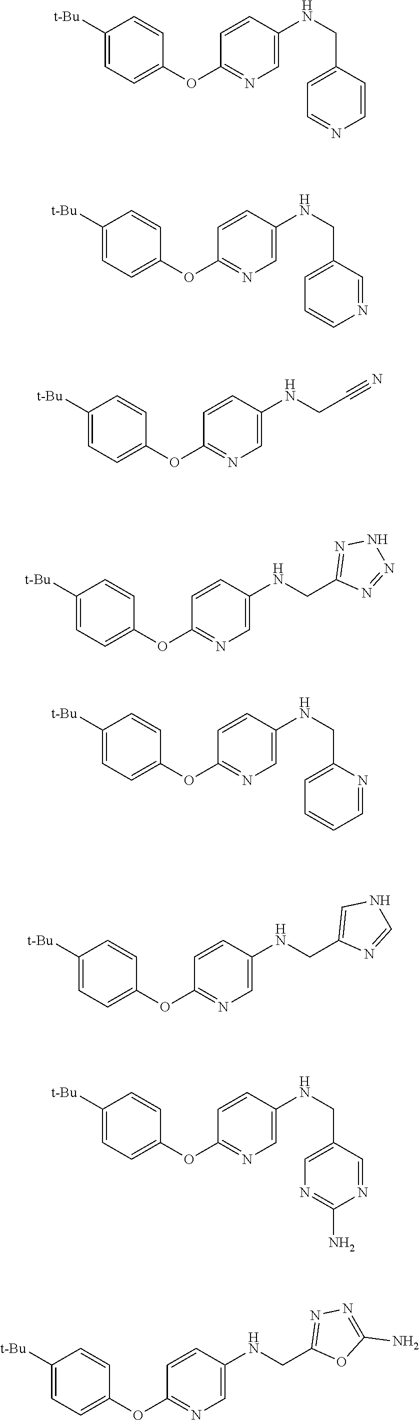

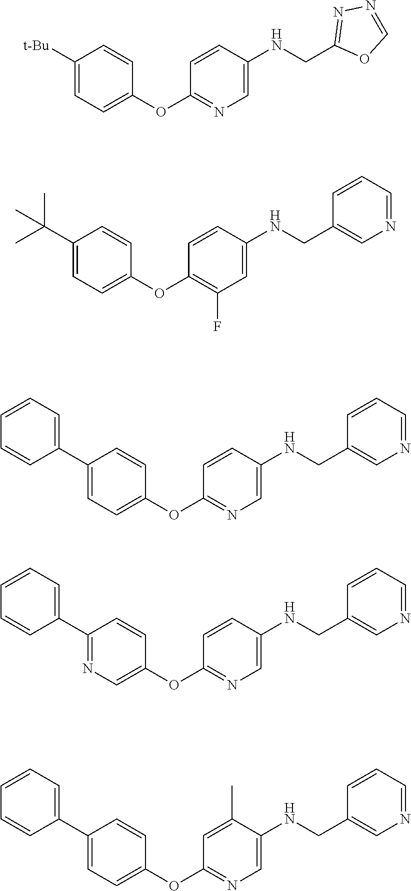 [SMILES: CC(C)(C)c1ccc(Oc2ccc(NCc3nnco3)cn2)cc1 ; CC(C)(C)c1ccc(Oc2ccc(NCc3cccnc3)cc2F)cc1 ; c1ccc(-c2ccc(Oc3ccc(NCc4cccnc4)cn3)cc2)cc1 ; c1ccc(-c2ccc(Oc3ccc(NCc4cccnc4)cn3)cn2)cc1 ; Cc1cc(Oc2ccc(-c3ccccc3)cc2)ncc1NCc1cccnc1]

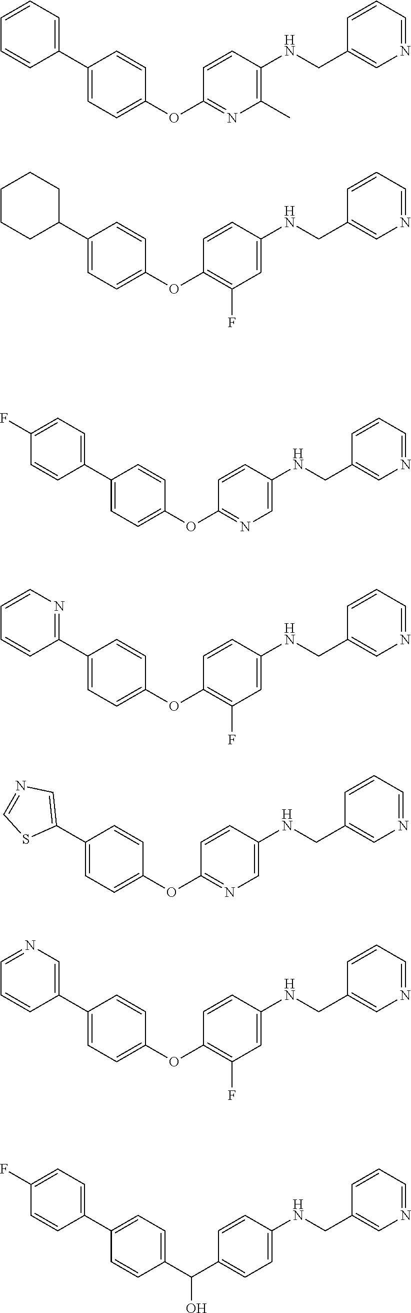

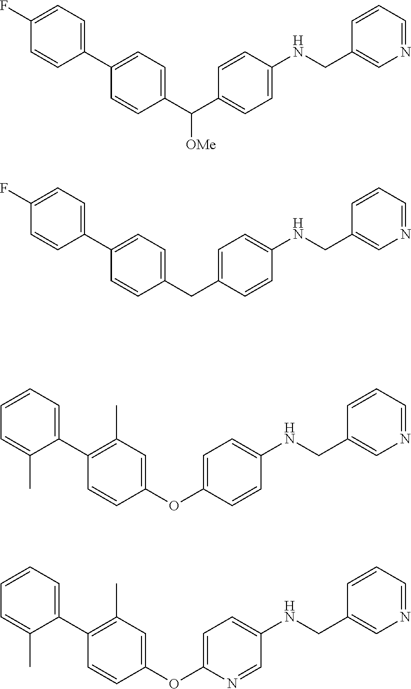 [SMILES: COC(c1ccc(NCc2cccnc2)cc1)c1ccc(-c2ccc(F)cc2)cc1 ; Fc1ccc(-c2ccc(Cc3ccc(NCc4cccnc4)cc3)cc2)cc1 ; Cc1ccccc1-c1ccc(Oc2ccc(NCc3cccnc3)cc2)cc1C ; Cc1ccccc1-c1ccc(Oc2ccc(NCc3cccnc3)cn2)cc1C]

A further most particular preferred embodiment of the invention are the following compounds pharmaceutically-acceptable salts, hydrates, solvates, or stereoisomers thereof:

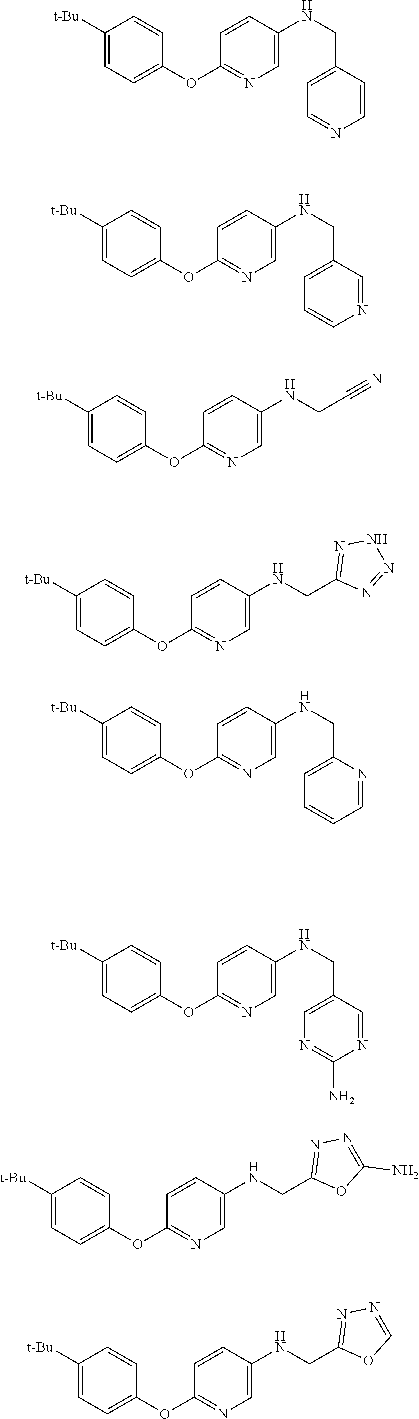

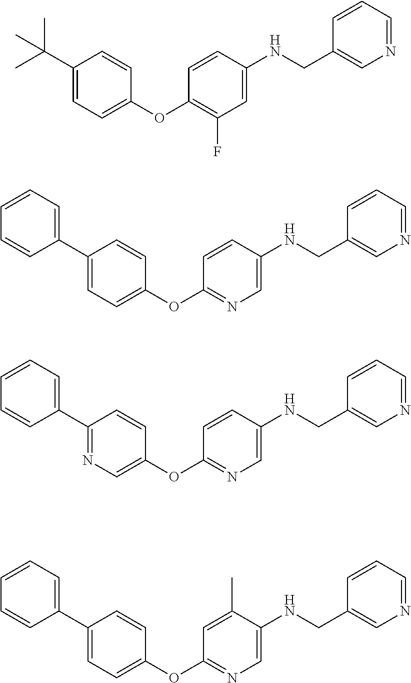 [SMILES: CC(C)(C)c1ccc(Oc2ccc(NCc3cccnc3)cc2F)cc1 ; c1ccc(-c2ccc(Oc3ccc(NCc4cccnc4)cn3)cc2)cc1 ; c1ccc(-c2ccc(Oc3ccc(NCc4cccnc4)cn3)cn2)cc1 ; Cc1cc(Oc2ccc(-c3ccccc3)cc2)ncc1NCc1cccnc1]

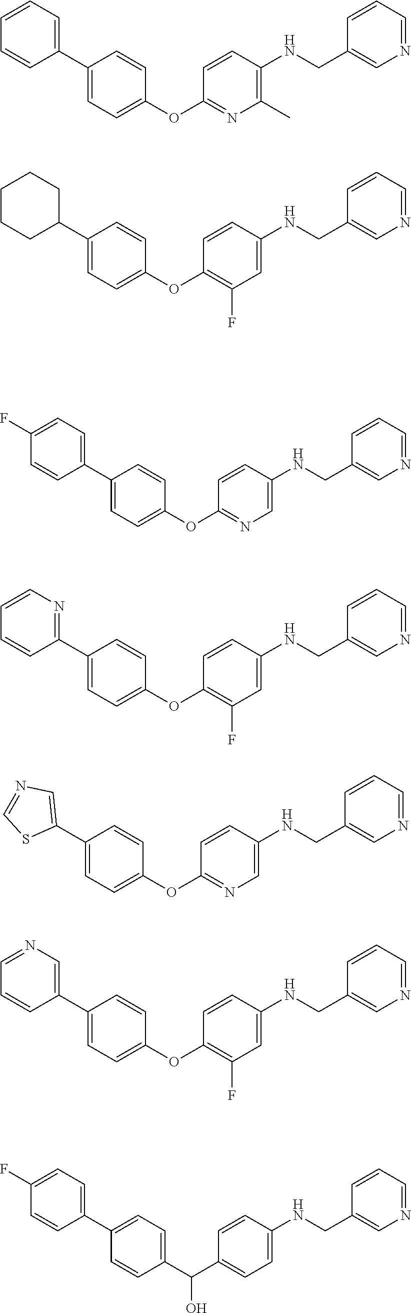

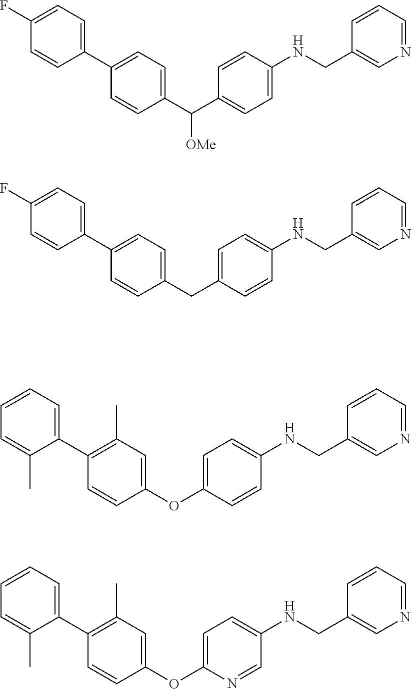 [SMILES: COC(c1ccc(NCc2cccnc2)cc1)c1ccc(-c2ccc(F)cc2)cc1 ; Fc1ccc(-c2ccc(Cc3ccc(NCc4cccnc4)cc3)cc2)cc1 ; Cc1ccccc1-c1ccc(Oc2ccc(NCc3cccnc3)cc2)cc1C ; Cc1ccccc1-c1ccc(Oc2ccc(NCc3cccnc3)cn2)cc1C]

A further most particular preferred embodiment of the invention are the following compounds pharmaceutically-acceptable salts, hydrates, solvates, or stereoisomers thereof:

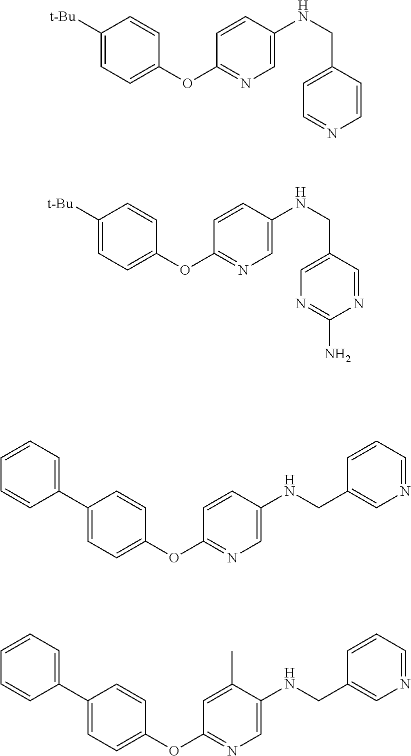 [SMILES: CC(C)(C)c1ccc(Oc2ccc(NCc3ccncc3)cn2)cc1 ; CC(C)(C)c1ccc(Oc2ccc(NCc3cnc(N)nc3)cn2)cc1 ; c1ccc(-c2ccc(Oc3ccc(NCc4cccnc4)cn3)cc2)cc1 ; Cc1cc(Oc2ccc(-c3ccccc3)cc2)ncc1NCc1cccnc1]

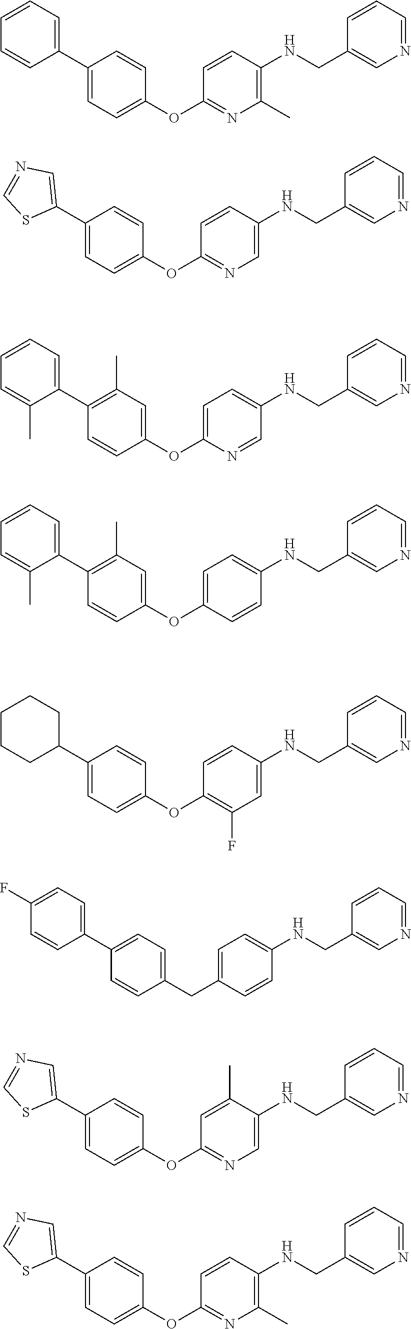

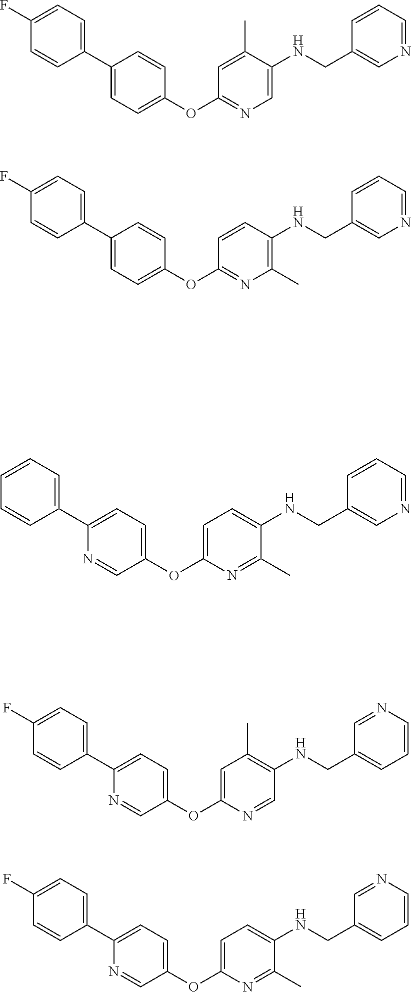 [SMILES: Cc1cc(Oc2ccc(-c3ccc(F)cc3)cc2)ncc1NCc1cccnc1 ; Cc1nc(Oc2ccc(-c3ccc(F)cc3)cc2)ccc1NCc1cccnc1 ; Cc1nc(Oc2ccc(-c3ccccc3)nc2)ccc1NCc1cccnc1 ; Cc1cc(Oc2ccc(-c3ccc(F)cc3)nc2)ncc1NCc1cccnc1 ; Cc1nc(Oc2ccc(-c3ccc(F)cc3)nc2)ccc1NCc1cccnc1]

A particular preferred embodiment of the invention are the following compounds pharmaceutically-acceptable salts, hydrates, solvates, or stereoisomers thereof:

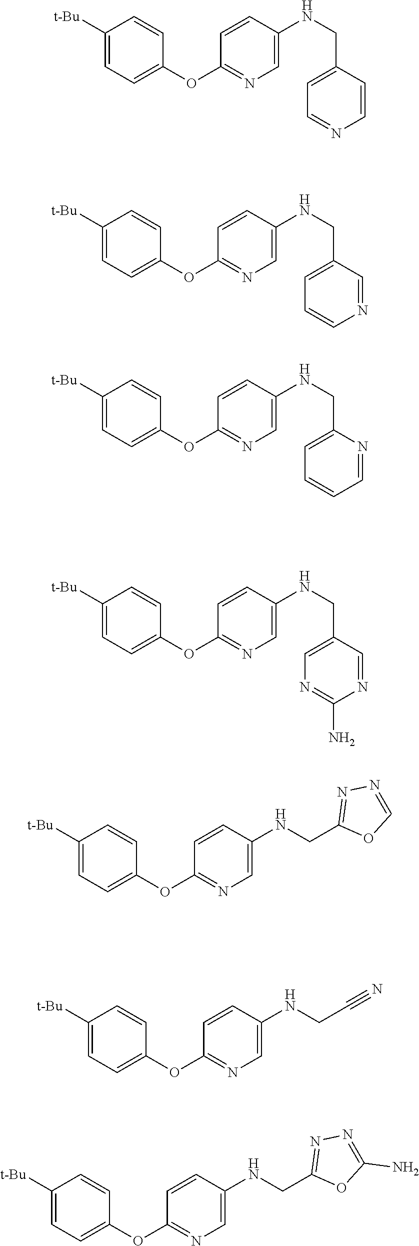

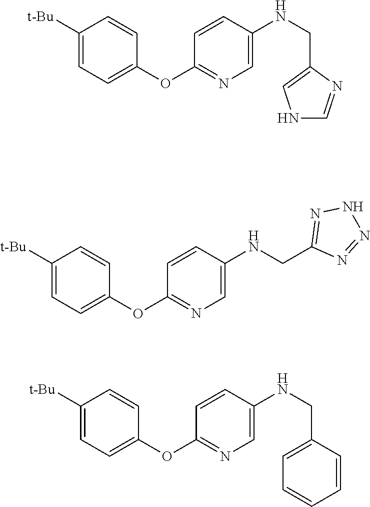 [SMILES: CC(C)(C)c1ccc(Oc2ccc(NCc3c[nH]cn3)cn2)cc1 ; CC(C)(C)c1ccc(Oc2ccc(NCc3nn[nH]n3)cn2)cc1 ; CC(C)(C)c1ccc(Oc2ccc(NCc3ccccc3)cn2)cc1]

The most particular preferred embodiment of the invention are the following compounds pharmaceutically-acceptable salts, hydrates, solvates, or stereoisomers thereof:

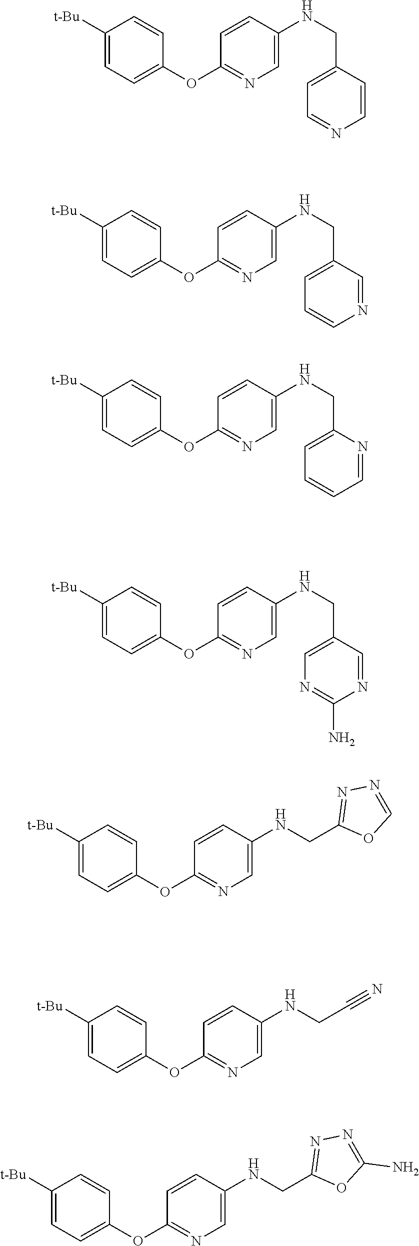

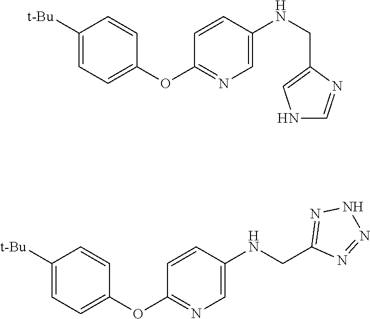 [SMILES: CC(C)(C)c1ccc(Oc2ccc(NCc3c[nH]cn3)cn2)cc1 ; CC(C)(C)c1ccc(Oc2ccc(NCc3nn[nH]n3)cn2)cc1]

Preparation of the Compounds

The compounds of the invention may be prepared by the exemplary processes described in the following reaction schemes or by the processes described in the examples. Exemplary reagents and procedures for these reactions appear hereinafter. Starting materials can be purchased or readily prepared by one of ordinary skilled in the art.

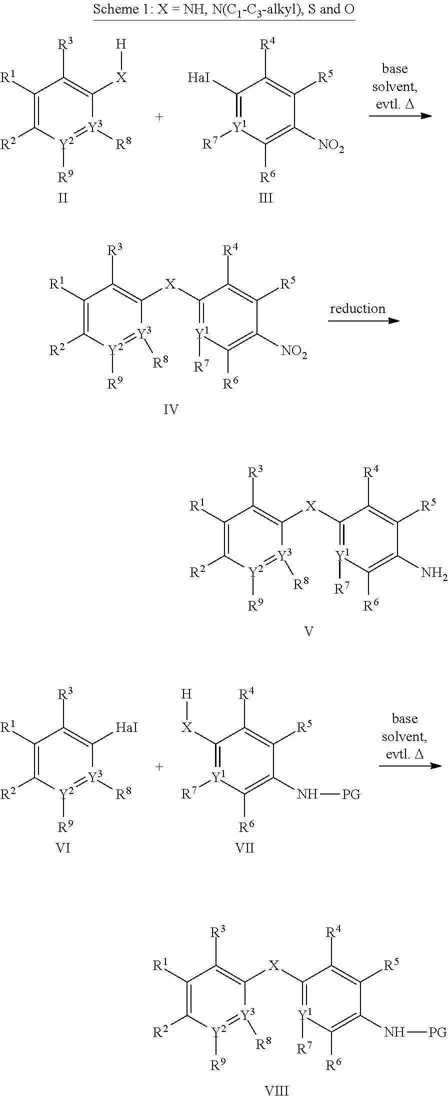

The syntheses of the di-arylamino, di-arylether and di-arylthioether analogs is depicted in Scheme 1: The respective amino-aryl moiety of formula (II) (X=NH, eventually monoalkylated) is reacted with the halogenated nitro-aryl precurser of formula (III) in a polar solvent in presence of a base at elevated temperatures. Preferably the solvent is a mixture of DMSO and an alcohol like tBuOH. As a base an alcoholate like tBuOK can be used. Reaction temperatures are between room temperature and 150° C., preferably between 60 and 110° C. The respective hydroxy- or mercapto-aryl moiety of formula (II) (X═O, S) is reacted with the halogenated nitro-aryl precurser of formula (III) in a polar solvent in presence of a base. A preferred reaction condition is carbonate as base in DMF at room temperature. Finally, the nitro function of formula (IV) can be reduced to the respective amine of formula (V) under Béchamp conditions or via catalytic hydrogenation. Preferred Bechamp conditions are Fe powder in a mixture of EtOH, H₂O and AcOH under sonication. Catalytic hydrogenation can be performed in presence of Pd/C in a polar solvent like an alcohol. Alternatively, target compounds of formula (VIII) (Y³=N) can be obtained via substitution of the halogene of formula (VI) by the X-containing aryl moiety of formula (VII), eventually in presence of a protection group (PG). Preferred conditions are phosphate as a base in an unpolar aromatic solvent at 100 to 150° C. under ferrocenyl catalysis [see: Advanced Synthesis & Catalysis 353 (2011), 3403].

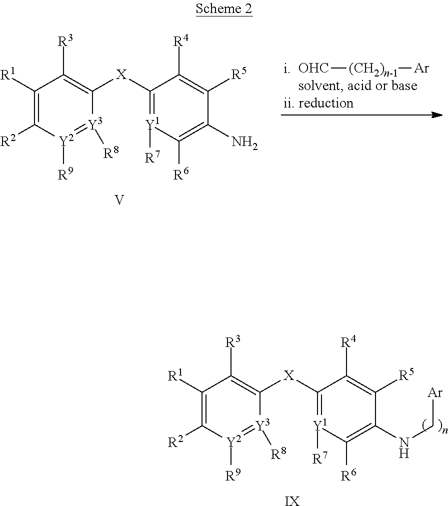

Scheme 2 depicts the reductive alkylation of amino-derivatives of formula (V): A preferred method is stirring the amine of formula (V) and the respective aldehyde in a polar solvent like an alcohol in presence of a weak acid like acetic acid. Then a reducing reagent like NaBH₃CN is added. Basic work-up finally yields compounds of formula (IX). Alternatively, the amine of formula (V) and the aldehyde are mixed in an unpolar solvent like dichloromethane in presence of a base like triethylamine. Then a reducing reagent like NaBH(OAc)₃ is added. Aqueous work-up finally yields compounds of formula (IX).

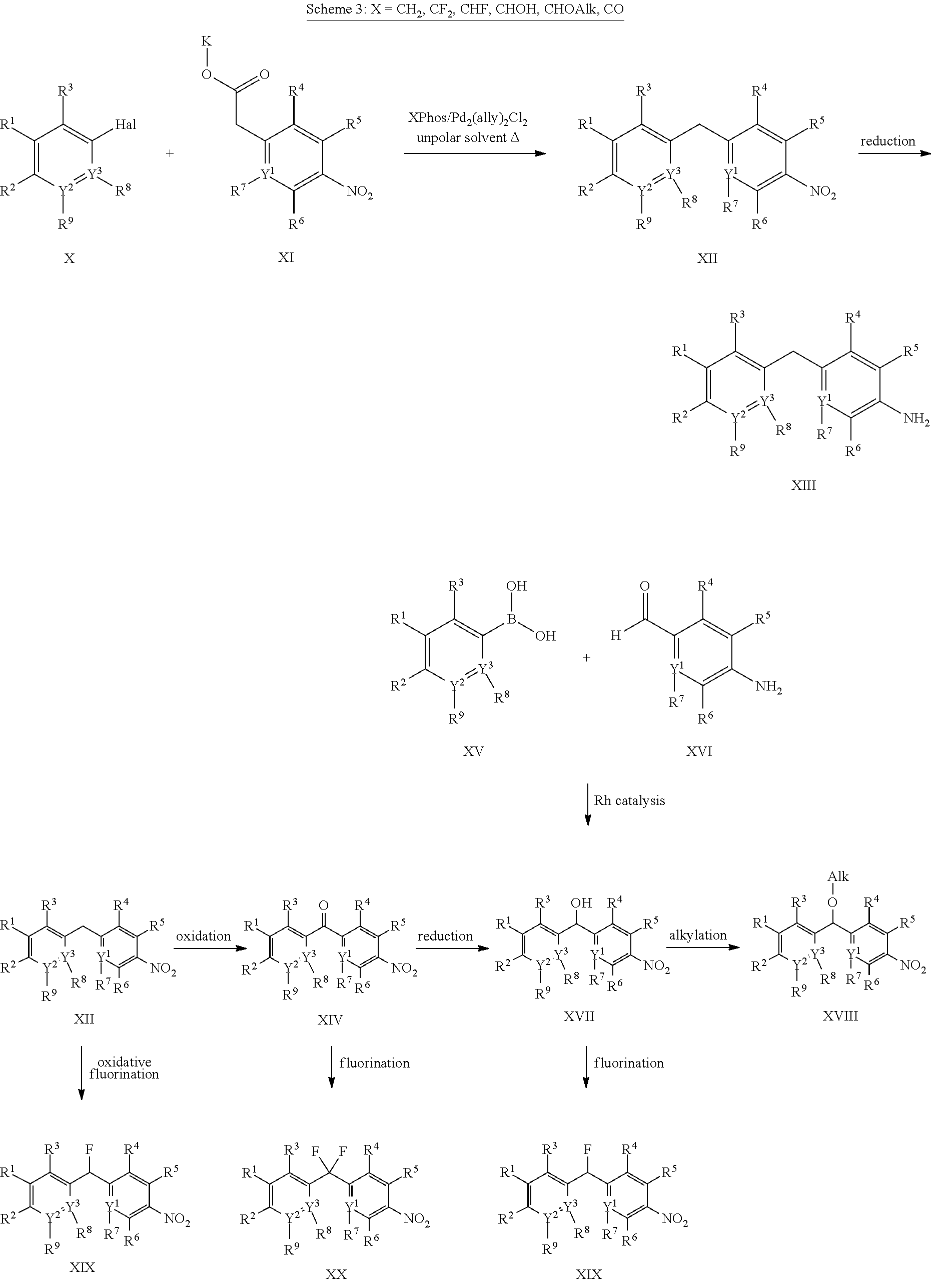

Scheme 3 illustrates the syntheses of carbon-bridged analogs (X═CH₂, CF₂, CHF, CHOH, CHOAlk, CO): The halogen-aryl moiety of formula (X) is decarboxylatively coupled to the aryl-acetate of formula (XI), catalyzed by a transition metal complex, yielding the nitro derivative of formula (XII). Preferred conditions are XPhos/Pd₂(allyl)₂Cl₂ as the catalyst in a unpolar solvent at elevated temperatures. The methylene bridge (X═CH₂) of formula (XII) can be oxidized to the respective di-aryl-ketone of formula (XIV). Preferred conditions are oxygen as the reagent in a mixture of acetic acid and DMSO at elevated temperature, catalyzed by FeCl₂.(H₂O)₄ [analogously to: Angew. Chem. Int. Ed. 51 (2012), 2745]. Reduction of the carbonyl group of formula (XIV) leads to the benzylic alcohol of formula (XVII). A preferred reducing reagent could be sodium borohydride. Alternatively, the benzylic alcohol of formula (XVII) can be obtained via cross coupling of a boronate of formula (XV) with an aryl-aldehyde of formula (XVI). Alkylation of the compound of formula (XVII) with an alkyl-iodide in presence of a strong base (e.g. NaH) in an aprotic polar solvent yields the alkoxy-derivative of formula (XVIII) [analogously to: Example 2 in U.S. Pat. No. 5,965,740]. The mono-fluoro derivative of formula (XIX) can be obtained either by oxidative fluorination of the compound of formula (XII) or hydroxy-substitution in a compound of formula (XVII). The oxidative fluorination can be done under conditions as Jacobsen salene complex, iodosylbenzene, base, tris(hydrogen fluoride) in a polar solvent at elevated temperatures [J. Am. Chem.Soc. 136 (2014), 6842]. Substitution of the benzylic hydroxy group by fluoride can be achieved by applying conditions like activation with trichloroacetimidate, 1,8-diazabicyclo[5.4.0]undec-7-ene in dichloromethane in presence of bis(1,5-cyclooctadiene)rhodium(I) tetrafluoroborate followed by triethylamine tris(hydrogen fluoride) in a mixture of F₃C—C₆H₅ and tetrahydrofurane at slightly elevated temperatures [Tetrahedron 71 (2015), 5932]. The keto-derivative of formula (XIV) can be converted to the difluoromethylen derivative of formula (XX) with [bis(2-methoxyethyl)amino]-sulfur trifluoride at elevated temperatures [analogously to: US2015246938; Step 2, Preparation of Compound 76; page 55]. Finally, the nitro-groups of compounds of formula (XII), (XIV) and (XVII)-(XX) can be reduced to the respective amino derivatives as described in Scheme 1.

Stereoisomers

Compounds of the present invention can exist as stereoisomers wherein asymmetric or chiral centers are present. These compounds are designated by the symbols“R” or“S”, depending on the configuration of sub stituents around the chiral carbon atom. The present invention contemplates various stereoisomers and mixtures thereof. Stereoisomers include enantiomers and diastereomers, and mixtures of enantiomers or diastereomers. Individual stereoisomers of compounds of the present invention can be prepared synthetically from commercially available starting materials which contain asymmetric or chiral centers or by preparation of racemic mixtures followed by resolution well-known to those of ordinary skill in the art.

These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns.

Geometric isomers can also exist in the compounds of the present invention. The present invention contemplates the various geometric isomers and mixtures thereof resulting from the arrangement of substituents around a carbon-carbon double bond or arrangement of substituents around a carbocyclic ring.

Compounds of the present invention can also exist as racemates which is given the descriptor “rac”. The term racemate, as used herein, means an equimolar mixture of a pair of enantiomers. A racemate is usually formed when synthesis results in the generation of a stereocenter. As used herein, the term racemic mixture means racemate. Compounds of the present invention can also exist as diastereomeric meso forms which is given the descriptor “rel”. The term diastereomeric meso form as used herein means achiral forms with a pseudostereogenic C-atom, which is given the descriptor “r” or “s”, respectively.

Salts

The compounds of the present invention may be used in the form of pharmaceutically-acceptable salts derived from inorganic or organic acids. By “pharmaceutically-acceptable salt”is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically-acceptable salts are well-known in the art. The salts may be prepared in situ during the final isolation and purification of the compounds of the invention or separately by reacting a free base function with a suitable acid.

Representative acid addition salts include, but are not limited to trifluoroacetic acid (TFA), acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides ; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically-acceptable basic addition salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Solvates/Hydrates

It should be appreciated that solvates and hydrates of the compound according to formula (I) are also within the scope of the present application. Methods of solvation are generally known in the art.

A further embodiment of the present invention may also include compounds, which are identical to the compounds of formula (I) except that one or more atoms are replaced by an atom having an atomic mass number or mass different from the atomic mass number or mass usually found in nature, e.g. compounds enriched in ²H (D), ³H, ¹³C, ¹²⁷I etc. These isotopic analogs and their pharmaceutical salts and formulations are considered useful agents in therapy and/or diagnosis, for example, but not limited to, where a fine-tuning of in vivo half-life time could lead to an optimized dosage regimen.

Pharmaceutical Compositions

In a further aspect the present invention provides a pharmaceutical composition comprising compounds of formula (I) according to the invention and a pharmaceutically acceptable diluent, excipient or carrier. In one embodiment the pharmaceutical composition further comprises another pharmaceutical active agent.

In one embodiment, the invention provides a pharmaceutical composition comprising a compound of formula (I) according to the invention and a pharmaceutically acceptable diluent, excipient or carrier, wherein said compound of formula (I) is present in a therapeutically effective amount.

Formulations and Modes of Administration

The compounds of the present invention may, in accordance with the invention, be administered in single or divided doses by oral, parenteral, inhalatory, rectal or topical administration including cutaneous, ophthalmic, mucosal scalp, sublingual, buccal and intranasal routes of administration; further, the compounds provided by the invention may be formulated to be used for the treatment of leukocyte populations ex vivo and in vitro.

When the compounds of the present invention are to be administered e.g. by the oral route, they may be administered as medicaments in the form of pharmaceutical compositions which contain them in association with a pharmaceutically acceptable diluent, excipient or carrier material. Thus the present invention also provides a pharmaceutical composition comprising the compounds according to the invention as described supra and one or more pharmaceutically acceptable diluent, excipient or carrier. The pharmaceutical compositions can be prepared in a conventional manner and finished dosage forms can be solid dosage forms, for example, tablets, dragees, capsules, and the like, or liquid dosage forms, for example solutions, suspensions, emulsions and the like. Pharmaceutically acceptable diluent, excipient or carrier include sterile aqueous solutions or dispersions and sterile powders for the extemporaneous preparation of sterile injectable solutions or dispersion. The use of such media and agents for pharmaceutically active substances is known in the art.

In one embodiment, the invention provides a pharmaceutical composition comprising a compound of formula (I) according to the invention and at least one pharmaceutically acceptable diluent, excipient or carrier, wherein the composition is a tablet or a capsule, preferably a tablet.

Dosing Regimen

An exemplary treatment regime entails administration once daily, twice daily, three times daily, every second day, twice per week, once per week. The composition of the invention is usually administered on multiple occasions. Intervals between single dosages can be, for example, less than a day, daily, every second day, twice per week, or weekly. The composition of the invention may be given as a continous uninterrupted treatment. In an exemplary treatment regime the compound of formula (I) according to the invention can be administered from 0.1-100 mg per day.

Therapeutic Use

The compounds according to the invention as described supra have preventive and therapeutic utility in human and veterinary diseases.

In one aspect of the present invention, the compounds according to the invention as described herein or the pharmaceutical composition as described herein may be used as a medicament, preferably for use in human medicine and/or veterinarian medicine. Accordingly the present invention provides the compounds according to the invention as described herein or a pharmaceutical composition as described herein, for use as a medicament.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prevention” comprises prophylactic treatments. In preventive applications, the pharmaceutical combination of the invention is administered to a subject suspected of having, or at risk for developing cancer. In therapeutic applications, the pharmaceutical combination is administered to a subject such as a patient already suffering from cancer, in an amount sufficient to cure or at least partially arrest the symptoms of the disease. Amounts effective for this use will depend on the severity and course of the disease, previous therapy, the subject's health status and response to the drugs, and the judgment of the treating physician.

The term “therapeutically effective amount” refers to an amount of a drug effective to treat a disease or disorder in a mammal. In the case of cancer, the therapeutically effective amount of the drug may reduce the number of tumor or cancer cells, reduce the tumor size; inhibit (i.e., slow to some extert and preferably stop) cancer cells infiltration into peripheral organs; inhibit (i.e., slow to some extert and preferably stop) tumor metastasis; inhibit, to some extert, tumor growth; and/or relieve to some extert one or more of the symptoms associated with the cancer. To the extert the compounds of the present invention may prevent growth and/or kill existing cancer cells, it may be cytostatic and/or cytotoxic.

The term “therapeutically effective amount” as used herein means an amount sufficient to prevent, or preferably reduce by at least about 30 percent, preferably by at least 50 percent, preferably by at least 70 percent, preferably by at least 80 percent, preferably by at least 90%, a clinically significant change in the growth or progression or mitotic activity of a target cellular mass, group of cancer cells, or other feature of pathology.

When provided preventively, compounds of the invention are provided in advance of established disease. The preventive administration of a compound of the present invention serves to prevent or attenuate the evolution of disease. The therapeutic administration of a compound of the present invention serves to attenuate established disease. Thus, in accordance with the invention, a compound of the present invention can be administered either prior to the onset of disease or during the course of disease.

In one aspect of the invention, there are provided the compounds of formula (I) according to the invention for use in a method for the prevention or treatment of oncovirus induced cancer in a subject.

Also provided is the use of the compounds according to the invention as described herein or the pharmaceutical composition as described herein for the manufacture of a medicament for the prevention or treatment of oncovirus induced cancer in a subject.

Also provided is the use of the compounds according to the invention as described herein or the pharmaceutical composition as described herein for the prevention or treatment of oncovirus induced cancer in a subject.

Also provided is a method for the prevention or treatment of oncovirus induced cancer in a subject, comprising administering to said subject a therapeutically effective amount of the compounds according to the invention as described herein or the pharmaceutical composition as described herein.

In a preferred embodiment the oncovirus inducing cancer is selected from Epstein-Barr Virus (EBV), Kaposi's Sarcoma Herpervirus (KSHV), Human Papillomavirus (HPV), Hepatitis B virus (HBV), Hepatitis C virus (HCV), Human T Cell Lymphotropic Virus-1 (HTLV-1) and Human Immunodeficiency Virus (HIV).

In an even more preferred embodiment the oncovirus inducing cancer is Epstein-Barr Virus (EBV) or Kaposi's Sarcoma Herpervirus (KSHV), in particular Epstein-Barr Virus (EBV). In a particular preferred embodiment the oncovirus induced cancer is selected from the group consisting of angio-immunoblastic T cell lymphomas, T/NK cell lymphomas, Burkitt lymphoma, classical Hodgkin lymphoma, post-transplant lymphoproliferative disorder (PTLD), non-Hodgkin lymphoma (NHL), Nasopharyngeal Carcinoma (NPC), lympho-epithelioma like gastric carcinomas, gastric adenocarcinomas, leiomypsarcomas, X-linked lymphoproliferative dieases, AIDS related lymphoproliferative disease, AIDS-related Kaposi's Sarcoma, classical Kaposi's Sarcoma (KS), Primary Effusion Lymphoma (PEL), Multicentric Castleman's disease (MCD), preferably oncovirus induced cancer selected from the group consisting of Burkitt lymphoma, classical Hodgkin lymphoma, post-transplant lymphoproliferative disorder (PTLD), non-Hodgkin lymphoma (NHL), Nasopharyngeal Carcinoma (NPC), lympho-epithelioma like gastric carcinomas, gastric adenocarcinomas, AIDS related lymphoproliferative disease, AIDS-related Kaposi's Sarcoma, classical Kaposi's Sarcoma (KS), Primary Effusion Lymphoma (PEL), Multicentric Castleman's disease (MCD), angio-immunoblastic T cell lymphomas, T/NK cell lymphomas, more preferably oncovirus induced cancer selected from the group consisting of Burkitt lymphoma, classical Hodgkin lymphoma, post-transplant lymphoproliferative disorder (PTLD), non-Hodgkin lymphoma (NHL), Nasopharyngeal Carcinoma (NPC), lympho-epithelioma like gastric carcinomas, gastric adenocarcinomas.

In a further aspect the present invention provides a kit of parts comprising a container and a package insert, wherein the first container comprises at least one dose of a medicament comprising a compound of formula (I) or a pharmaceutically-acceptable salt, hydrate, solvate, or stereoisomer thereof and optionally one or more pharmaceutically acceptable diluents, excipients or carrier, and the package insert comprises instructions for treating a subject for oncovirus induced cancer using the medicament.

In a further aspect the present invention provides the use of the compound of formula (I) or pharmaceutically-acceptable salt, hydrate, solvate, or stereoisomer thereof or a compound as described supra or a pharmaceutically-acceptable salt, hydrate, solvate, or stereoisomer thereof or the kit as described supra in diagnosing, predicting, and/or monitoring of oncovirus induced cancer in a subject.

### EXAMPLES

### Chemical Synthesis of Compounds

Abbreviations

AcOH acetic acid

brine saturated aqueous NaCl solution

CV column volumes

DCE 1,2-dichlorethane

DCM dichloromethane

DME 1,2-dimethoxyethane

DMF dimethylformamide

DMSO-d6 deuterated dimethyl sulfoxide

equiv equivalent(s)

EtOAc ethyl acetate

Et₂O diethyl ether

EtOH ethanol

expl. example

Fe iron

h hour(s)

HCl hydrochloric acid

M molar concentration

MeOH methanol

MgSO₄ magnesium sulfate

min minute(s)

mL milliliter(s)

Mw molecular weight

NaBH(OAc)₃ sodium triacetoxyborohydride

NaHCO₃ sodium bicarbonate

Na₂SO₄ sodium sulfate

Pd/C palladium on carbon

pTSA p-toluenesulfonic acid

RT room temperature

tBuOH tert.-butanol

tBuOK potassium tert.-butylate

TEA triethylamine

THF tetrahydrofurane

TLC thin layer chromatography (R_{f}. retertion factor)

### General Procedure A: Aromatic Nucleophilic Substitution with Substituted Phenol (Refers to Scheme 1)

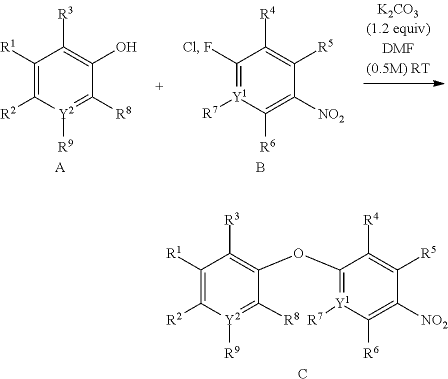

To the desired aryl alcohol A (1.1 equiv) and the corresponding 4-halo-nitroaryl B (1.0 equiv) in DMF (0.5 M), was added K₂CO₃ (1.2 equiv). The reaction was stirred at RT. After completion (monitored by TLC with EtOAc/hexanes or EtOAc/cyclohexane as eluent and stained with KMnO₄), usually observed after 14 h, the reaction mixture was poured into a mixture of Et₂O and a satured aqueous solution of NaHCO₃. The layers were separated and the aqueous phase extracted twice with Et₂O. The combined organic layers were washed with a saturated aqueous solution of NaHCO₃, dried over Na₂SO₄ or MgSO₄, filtered-off and concentrated under reduced pressure. The crude product was purified by combi flash column chromatography using EtOAc/hexanes or EtOAc/cyclohexane as the eluent, to afford the corresponding title compound C.

### General Procedure B: Nitro-Aromatic Reduction (Refers to Scheme 1)

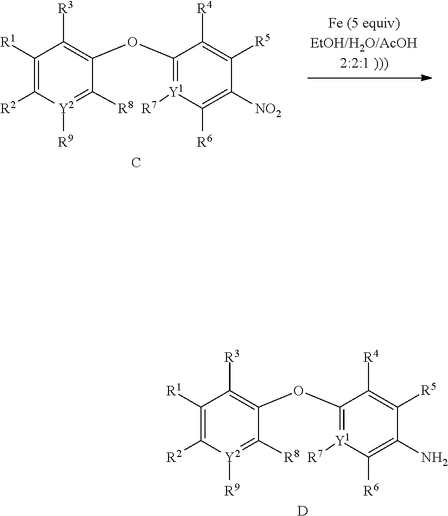

To the nitro compound C (1.0 equiv) were added Fe powder (5.0 equiv) and EtOH/H₂O/AcOH 2:2:1 (0.1 M). The reaction was sonicated until completion (monitored by TLC, with EtOAc/hexanes or EtOAc/cyclohexane as eluent and stained with KMnO₄). The obtained brown slurry was filtered through filter paper, rinsed with EtOAc and the organic solvents were evaporated. EtOAc was added followed by careful addition of a saturated aqueous solution of NaHCO₃. Layers were separated and the aqueous layer was extracted three times with EtOAc. The combined organic layers were dried over MgSO₄ or Na₂SO₄, filtered-off and the solvent was evaporated. The crude product was purified by combi flash column chromatography using EtOAc/hexanes or EtOAc/cyclohexane as the eluent, to afford the corresponding title compound D.

### General Procedure C: Reductive Amination of the Arylamine Hydrochloride (Refers to Scheme 2)

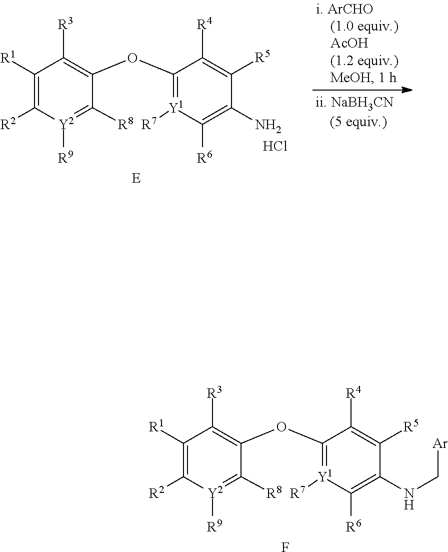

To the HCl salt of the arylamine (E) (1.0 equiv) and the aldehyde (1.0 equiv) in MeOH (0.18 M) under inert atmosphere, was added AcOH (1.2 equiv). After 1 h stirring, NaBH₃CN (5.0 equiv) was added. This mixture was stirred until completion (monitored by TLC, with EtOAc/hexanes or EtOAc/cyclohexane as eluent and stained with KMnO₄). The reaction was then neutralized by dropwise addition of a 1.0 M aqueous solution of NaOH and the solvent was evaporated. The obtained residue was poured into EtOAc and a 1.0 M aqueous solution of NaOH. The layers were separated and the aqueous layer was extracted twice with EtOAc. The combined organic layers were dried over MgSO₄ or Na₂SO₄, filtered-off and solvent was evaporated. The crude product was purified by combi flash column chromatography using EtOAc/hexanes or EtOAc/cyclohexane as the eluent, to afford the corresponding title compound F.

General Procedure D: Suzuki Coupling

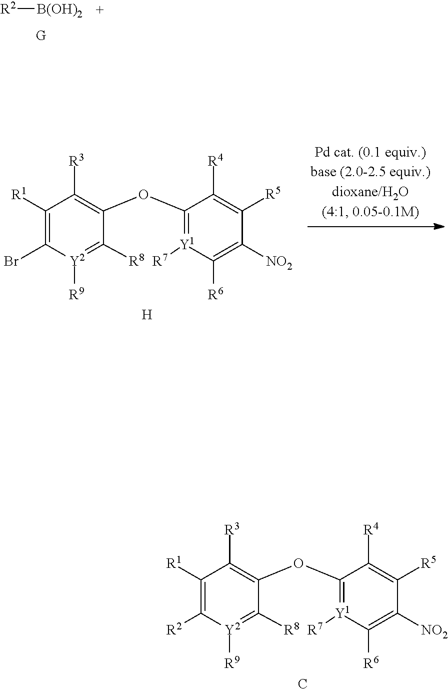

To a suspension of the desired boronic acid G (1.2 equiv), the desired bromoaryl H (1.0 equiv) and the accurate base (2.0-2.5 equiv) in dioxane/H₂O 4:1 (0.05-0.1 M), the palladium catalyst (10% mol) was added. The reaction mixture was stirred at reflux. After 16 h, EtOAc and H₂O were added. The layers were separated and the aqueous layer was extracted with EtOAc (2×). The combined organic layers were washed with brine, dried over MgSO₄ or Na₂SO₄, filtered through a pad of celite and concentrated under reduced pressure. The residue was purified by column chromatography using EtOAc/hexanes or EtOAc/cyclohexane as the eluent, to afford the target compound C.

N.B.: This procedure was also applied to Suzuki couplings between G as a bromoaryl derivative and H as aryl boronic acid/ester.

### General Procedure E: Suzuki Coupling

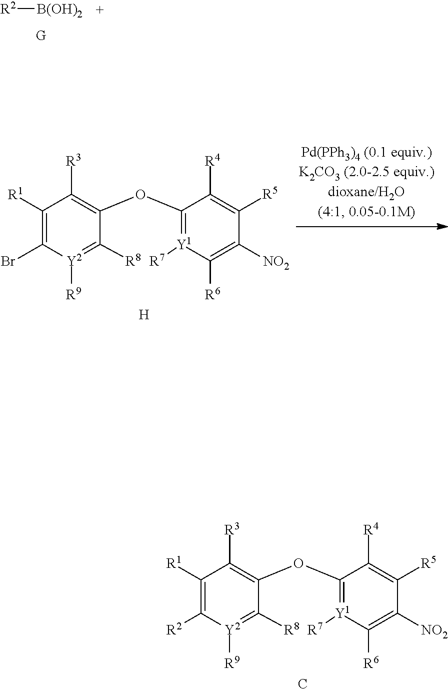

To a suspension of the desired boronic acid G (1.2 equiv), the desired bromoaryl H (1.0 equiv) and K₂CO₃ (2.0-2.5 equiv) in dioxane: H₂O 4:1 (0.05-0.1 M), tetrakis(triphenylphosphine)palladium(0) (10% mol) was added. The reaction mixture was stirred at reflux. After 16 h, EtOAc and H₂O were added. The layers were separated and the aqueous layer was extracted with EtOAc (2×). The combined organic layers were washed with brine, dried over MgSO₄ or Na₂SO₄, filtered through a pad of celite and concentrated under reduced pressure. The residue was purified by column chromatography using EtOAc/hexanes or EtOAc/cyclohexane as the eluent, to afford the target compound C.

### General Procedure F: Pyridyl Reductive Amination (Refers to Scheme 2)

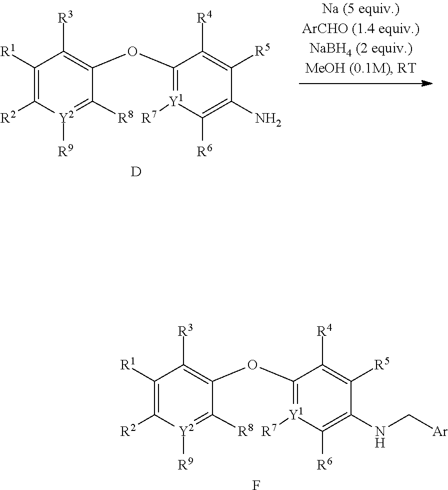

To a freshly prepared solution of sodium methoxide (Na 5.0 equiv, MeOH 0.1M) under inert atmosphere, the aminoaryl derivative D (1.0 equiv) was added. The reaction was stirred at RT (1 h). Then, the appropriate aldehyde (1.4 equiv) was added followed, after 16 h, by NaBH₄ (2.0 equiv). The mixture was stirred until completion (monitored by TLC). MeOH was evaporated and EtOAc followed by saturated aqueous NaHCO₃ were added. The layers were separated and the organic layer was washed with brine, dried over MgSO₄ or Na₂SO₄, filtered and evaporated under vacuum. The crude residue was purified by column chromatography using EtOAc/hexanes or EtOAc/cyclohexane as the eluent, to afford the target compound F

### General Procedure G: Nitro-Aromatic Reduction (Refers to Scheme 1)

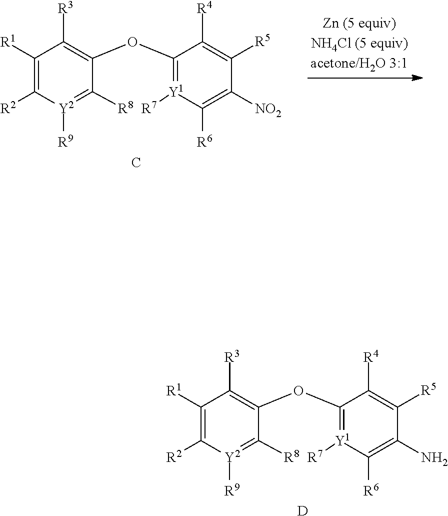

To a solution of the nitro compound C (1.0 equiv), at RT, in a 3:1 mixture of acetone/H₂O was added NH₄Cl (5 equiv). To this stirring solution Zn (5.0 equiv) was added in portions. The reaction mixture was stirred for 1 h (monitored by TLC, with EtOAc/hexanes or EtOAc/cyclohexane as eluent and stained with KMnO₄) and then concentrated under reduced pressure. The residue was suspended in EtOAc and filtered through a pad of celite which was washed with EtOAc. The filtrate was washed washed with NaHCO₃ (2×), dried over MgSO₄ or Na₂SO₄, filtered-off and the solvent was evaporated to afford the corresponding title compound D.

### General Procedure H: Pyridyl Reductive Amination (Refers to Scheme 2)

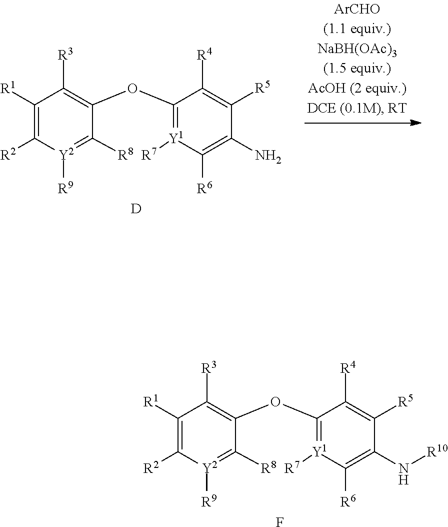

To a solution of the aminoaryl derivative D (1.0 equiv), in DCE (0.25M), at RT, was added the appropriate aldehyde (1.1 equiv). The mixture was stirred for 5 min before NaBH(OAc)₃ (1.5 equiv) was added followed by AcOH (1 equiv) addition. The reaction was stirred at RT overnight. The reaction mixture was quenched by adding 1M NaOH and diluted with H₂O and CH₂Cl₂. The two layers were separated and the aqueous layer was extracted with CH₂Cl₂ (2×). The combined organic layers were washed with brine, dried over MgSO₄ or Na₂SO₄, filtered and evaporated under vacuum. The crude residue was purified by column chromatography using EtOAc/hexanes or EtOAc/cyclohexane as the eluent, to afford the target compound F.

### Example 1

### 6-(4-(tert-Butyl)phenoxy)-N-(pyridin-4-ylmethyl)pyridin-3-amine

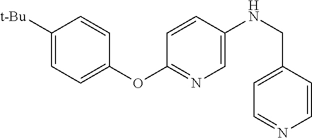 [SMILES: CC(C)(C)c1ccc(Oc2ccc(NCc3ccncc3)cn2)cc1]

Following the General procedure C, 6-(4-(tert-butyl)phenoxy)-N-(pyridin-4-ylmethyl)pyridin-3-amine was obtained in 72% yield (0.31 mmol, 103 mg) from the HCl salt of 6-(4-(tert-butyl)phenoxy)pyridin-3-amine (0.043 mmol, 118 mg).

C₂₁H₂₃N₃O; Mw=333.44 g.mol⁻¹; Yellowish sticky oil; ¹H NMR (400 MHz, CDCl₃) δ 8.60-8.52 (m, 2H), 7.61 (d, J=3.0 Hz, 1H), 7.38-7.31 (m, 2H), 7.31-7.27 (m, 2H), 7.01-6.92 (m, 3H), 6.76 (d, J=8.8 Hz, 1H), 4.36 (s, 2H), 4.09 (s, 1H), 1.30 (s, 9H); ¹³C NMR (101 MHz, CDCl₃) δ 156.73, 150.27, 148.28, 139.95, 132.21, 126.59, 124.80, 122.18, 119.52, 112.52, 77.48, 77.16, 76.84, 47.74, 34.47, 31.63.

The starting material was prepared as follows:

### Step 1: 2-(4-(tert-Butyl)phenoxy)-5-nitropyridine

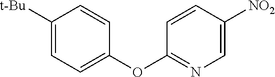 [SMILES: CC(C)(C)c1ccc(Oc2ccc([N+](=O)[O-])cn2)cc1]

Following the General procedure A, 2-(4-(tert-butyl)phenoxy)-5-nitropyridine was obtained in 72% yield (14.41 mmol, 3.92 g) from 4-(tert-butyl)phenol (19.97 mmol, 3.00 g) and 2-chloro-5-nitropyridine (19.97 mmol, 3.17 g).

C₁₅H₁₆N₂O₃; Mw=272.30 g.mol⁻¹; Yellow oil; ¹H NMR (400 MHz, CDCl₃) δ 9.07 (dd, J=2.8, 0.5 Hz, 1H), 8.46 (dd, J=9.1, 2.8 Hz, 1H), 7.50-7.39 (m, 2H), 7.11-7.05 (m, 2H), 7.01 (dd, J=9.1, 0.5 Hz, 1H), 1.36 (s, 9H).

### Step 2: 6-(4-(tert-Butyl)phenoxy)pyridin-3-amine

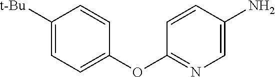 [SMILES: CC(C)(C)c1ccc(Oc2ccc(N)cn2)cc1]

6-(4-(tert-Butyl)phenoxy)pyridin-3-amine was obtained following the General procedure B.

C₁₅H₁₈N₂O; Mw=242.32 g.mol⁻¹; ¹H 1NMR (400 MHz, CDCl₃) δ 7.69 (d, J=3.0 Hz, 1H), 7.39-7.3 1 (m, 2H), 7.03 (dd, J=8.6, 3.0 Hz, 1H), 7.00-6.93 (m, 2H), 6.72 (d, J=8.6 Hz, 1H), 3.48 (s, NH₂), 1.31 (s, 9H).

### Example 2

### 6-(4-(tert-Butyl)phenoxy)-N-(pyridin-3-ylmethyl)pyridin-3-amine

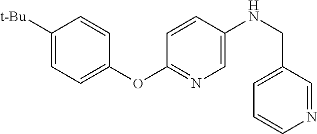 [SMILES: CC(C)(C)c1ccc(Oc2ccc(NCc3cccnc3)cn2)cc1]

Following the General procedure C, 6-(4-(tert-butyl)phenoxy)-N-(pyridin-3-ylmethyl)pyridin-3-amine was obtained in 86% yield (0.90 mmol, 300 mg) from the HCl salt of 6-(4-(tert-butyl)phenoxy)pyridin-3-amine (1.05 mmol, 291 mg).

C₂₁H₂₃N₃O; Mw=333.44 g.mol⁻¹; Yellowish sticky oil; ¹H NMR (400 MHz, CDCl₃) δ 8.63 (s, 1H), 8.54 (d, J=3.7 Hz, 1H), 7.67 (dd, J=12.7, 5.4 Hz, 2H), 7.39-7.31 (m, 2H), 7.31-7.26 (m, 1H), 7.04-6.94 (m, 3H), 6.77 (d, J=8.7 Hz, 1H), 4.34 (s, 2H), 4.07-3.88 (m, 1H), 1.31 (s, 9H); ¹³C NMR (101 MHz, CDCl₃) δ 156.67, 153.29, 149.28, 149.15, 146.51, 140.15, 135.27, 134.33, 132.28, 126.58, 124.95, 123.76, 119.46, 112.57, 77.48, 77.16, 76.84, 46.48, 34.46, 31.63.

### Example 3

### 6-(4-(tert-Butyl)phenoxy)-N-(pyridin-2-ylmethyl)pyridin-3-amine

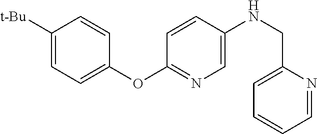 [SMILES: CC(C)(C)c1ccc(Oc2ccc(NCc3ccccn3)cn2)cc1]

Following the General procedure C, 6-(4-(tert-butyl)phenoxy)-N-(pyridin-2-ylmethyl)pyridin-3-amine was obtained in 65% yield (0.23 mmol, 75 mg) from the HCl salt of 6-(4-(tert-butyl)phenoxy)pyridin-3-amine (0.36 mmol, 100 mg).

C₂₁H₂₃N₃O; Solid; Mw=333.44 g.mol⁻¹; ¹H NMR (400 MHz, CDCl₃) δ 8.59 (d, J=4.3 Hz, 1H), 7.71 (d, J=3.0 Hz, 1H), 7.67 (td, J=7.7, 1.8 Hz, 1H), 7.34-7.30 (m, 3H), 7.21 (dd, J=7.0, 5.1 Hz, 1H), 7.07 (d, J=8.7, 3.1 Hz, 1H), 7.01-6.95 (m, 2H), 6.78 (d, J=8.7 Hz, 1H), 4.43 (s, 2H), 1.31 (s, 9H).

### Example 4

### N-Benzyl-6-(4-(tert-butyl)phenoxy)pyridin-3-amine

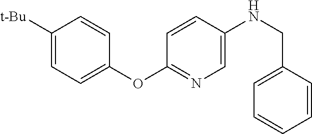 [SMILES: CC(C)(C)c1ccc(Oc2ccc(NCc3ccccc3)cn2)cc1]

Following the General procedure C, N-benzyl-6-(4-(tert-butyl)phenoxy)pyridin-3-amine was obtained in 17% yield (0.168 mmol, 56 mg) from the HCl salt of 6-(4-(tert-butyl)phenoxy)pyridin-3-amine (0.984 mmol, 274 mg).

C₂₂H₂₄N₂O; Mw=332.45 g.mol⁻¹; White solid; ¹H NMR (400 MHz, CDCl₃) δ 7.69 (d, J=3.0 Hz, 1H), 7.39-7.26 (m, 7 H), 7.05-6.94 (m, 3H), 6.76 (d, J=8.7 Hz, 1H), 4.31 (s, 2H), 1.31 (s, 9H); ¹³C NMR (101 MHz, CDCl₃) δ 156.27, 153.49, 146.36, 140.73, 138.81, 132.26, 128.89, 127.64, 127.63, 126.56, 124.72, 119.34, 112.55, 77.48, 77.16, 76.84, 48.95, 34.45, 31.64.

### Example 5

### 5-(((6-(4-(tert-Butyl)phenoxy)pyridin-3-yl)amino)methyl)pyrimidin-2-amine

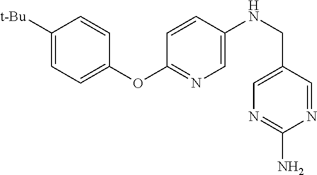 [SMILES: CC(C)(C)c1ccc(Oc2ccc(NCc3cnc(N)nc3)cn2)cc1]

To a suspension of the HCl salt of 6-(4-(tert-butyl)phenoxy)pyridin-3-amine (1.0 equiv, 1.26 mmol, 350 mg) and 2-aminopyrimidine-5-carbaldehyde (0.95 equiv, 1.19 mmol, 150 mg) in CH₂Cl₂ (0.2 M, 6.0 mL) under inert atmosphere, was added triethylamine (2.2 equiv, 2.76 mmol, 0.38 mL). The brown suspension was stirred for 1.5 h and NaBH(OAc)₃ (2.95 equiv, 3.71 mmol, 810 mg) was added. The so obtained beige suspension was then stirred for 4 h.

The reaction was poured into ice and EtOAc and the layers were separated. The aqueous layer was extracted with EtOAc. The combined organic layers were dried over Na₂SO₄, filtered-off and concentrated. The crude product was purified by combi flash column chromatography using Et0Ac/MeOH as the eluent, to afford 5-(((6-(4-(tert-butyl)phenoxy)pyridin-3-yl)amino)methyl)pyrimidin-2-amine (0.48 mmol, 166 mg, 38% yield).

C₂₀H₂₃N₅O; Mw=349.44 g.mol⁻¹; ¹H NMR (400 MHz, DMSO-d₆) δ 8.23 (s, 2H), 7.55 (d, J=2.9 Hz, 1H), 7.34 (d, J=8.7 Hz, 2H), 7.13 (dd, J=8.7, 3.0 Hz, 1H), 6.88 (d, J=8.7 Hz, 2H), 6.79 (d, J=8.7 Hz, 1H), 6.53 (s, 2H), 6.03 (t, J=5.9 Hz, 1H), 4.03 (d, J=5.8 Hz, 2H), 1.27 (s, 9H).

### Example 6

### 2-((6-(4-(tert-Butyl)phenoxy)pyridin-3-yl)amino)acetonitrile

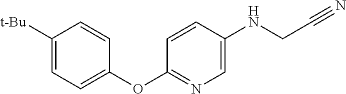 [SMILES: CC(C)(C)c1ccc(Oc2ccc(NCC#N)cn2)cc1]

To a suspension of the HCl salt of 6-(4-(tert-butyl)phenoxy)pyridin-3-amine (1.0 equiv, 1.04 mmol, 290 mg) and Cs₂CO₃ (2.5 equiv, 2.60 mmol, 847 mg) in DMF (0.4 M, 2.5 mL) under inert atmosphere, bromoacetonitrile (1.0 equiv, 1.04 mmol, 72 μL) was added. The yellow suspension was stirred 16 h at 80° C. The reaction mixture was poured into a stirred mixture of EtOAc and water. The layers were separated and the organic layer was washed with water, dried over Na₂SO₄ or MgSO₄, filtered-off and concentrated under reduced pressure. The crude product was purified by combi flash column chromatography using toluene/EtOAc as the eluent, to afford 2-((6-(4-(tert-butyl)phenoxy)pyridin-3-yl)amino)acetonitrile (0.10 mmol, 28 mg, 10% yield).

C₁₇H₁₉N₃O; Mw=281,36 g.mol⁻¹; ¹H NMR (400 MHz, DMSO-d₆) δ 7.78-7.75 (m, 1H), 7.40-7.36 (m, 2H), 7.14 (dd, J=8.8, 3.1 Hz, 1H), 7.04-6.99 (m, 2H), 6.87 (dd, J=8.7, 0.7 Hz, 1H), 4.10 (s, 2H), 1.32 (s, 9H).

### Example 7

### N-((2H-Tetrazol-5-yl)methyl)-6-(4-(tert-butyl)phenoxy)pyridin-3-amine

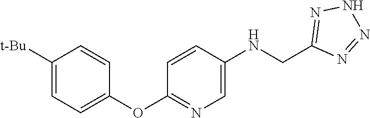 [SMILES: CC(C)(C)c1ccc(Oc2ccc(NCc3nn[nH]n3)cn2)cc1]

To a stirred 9:1 mixture of DMF and MeOH (0.4 M, 0.45 mL) in a microwave tube, 2-((6-(4-(tert-butyl)phenoxy)pyridin-3-yl)amino)acetonitrile (1.0 equiv, 0.17 mmol, 44 mg), copper(I) bromide (0.07 equiv, 12 μmol, 1.7 mg) and azidotrimethylsilane (1.5 equiv, 0.26 mmol, 34 μL) were added. The tube was sealed and N₂ was bubbled through the mixture. The yellow suspension was stirred 16 h at 85° C. The reaction mixture was poured into a stirred mixture of EtOAc and water. The layers were separated and the organic layer was washed with water, dried over Na₂SO₄ or MgSO₄, filtered-off and concentrated under reduced pressure. The crude product was purified by combi flash column chromatography using CH₂Cl₂/MeOH as the eluent, to afford N-((2H-tetrazol-5-yl)methyl)-6-(4-(tert-butyl)phenoxy)pyridin-3-amine (0.07 mmol, 22 mg, 40% yield).

C₁₇H₂₀N₆O; Mw=324.39 g.mol⁻¹; ¹H NMR (400 MHz, DMSO-d₆) δ 7.57 (d, J=3.0 Hz, 1H), 7.37-7.32 (m, 2H), 7.15 (dd, J=8.7, 3.1 Hz, 1H), 6.91-6.86 (m, 2H), 6.82 (d, J=8.7 Hz, 1H), 6.35 (t, J=5.9 Hz, 1H), 4.59 (d, J=5.8 Hz, 2H), 1.27 (s, 9H).

### Example 8

### N-((1,3,4-Oxadiazol-2-yl)methyl)-6-(4-(tert-butyl)phenoxy)pyridin-3-amine

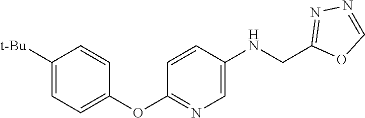 [SMILES: CC(C)(C)c1ccc(Oc2ccc(NCc3nnco3)cn2)cc1]

A solution of 2-((6-(4-(tert-butyl)phenoxy)pyridin-3-yl)amino)acetohydrazide (1.0 equiv, 350 μmol, 110 mg) and formic acid (1.0 equiv, 350 μmol, 13 μL) in DCE (0.3 M, 1 mL) in a sealed microwave vial was stirred 30 min, then POCl₃ (1.0 equiv, 350 μmol, 33 μL) was added. The yellow solution was stirred 16 h at 85° C. Volatiles were removed and the crude product was purified by combi flash column chromatography using cyclohexane/EtOAc as the eluent, to afford N-((1,3,4-oxadiazol-2-yl)methyl)-6-(4-(tert-butyl)phenoxy)pyridin-3-amine (43 μmol, 14 mg, 12% yield).

C₁₈H₂₀N₄O₂; Mw=324.38 g.mol⁻¹; ¹H I NMR (400 MHz, DMSO-d₆) δ 10.66 (s, 1H), 8.05 (d, J=2.6 Hz, 1H), 7.78 (dd, J=9.0, 3.1 Hz, 1H), 7.47 (s, 1H), 7.43-7.39 (m, 2H), 7.05 (dd, J=8.9, 0.5 Hz, 1H), 7.03-6.99 (m, 2H), 4.19 (s, 2H), 1.30 (s, 9H).

The starting material was prepared as follows:

### Step 1: Methyl (6-(4-(tert-butyl)phenoxy)pyridin-3-yl)glycinate

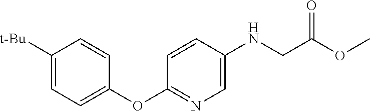 [SMILES: COC(=O)CNc1ccc(Oc2ccc(C(C)(C)C)cc2)nc1]

To a suspension of the HCl salt of 6-(4-(tert-butyl)phenoxy)pyridin-3-amine (1.0 equiv, 3.62 mmol, 1.0 g) and K₂CO₃ (1.3 equiv, 4.60 mmol, 640 mg) in DMF (0.3 M, 12 mL) under inert atmosphere, bromomethyl acetate (1.3 equiv, 4.60 mmol, 0.45 mL) was added. The pink suspension was stirred 60 h at RT. The reaction mixture was poured into a stirred mixture of EtOAc and water. The layers were separated and the organic layer was washed with water, dried over Na₂SO₄ or MgSO₄, filtered-off and concentrated under reduced pressure. The crude product was purified by combi flash column chromatography using cyclohexane/EtOAc as the eluent, to afford methyl (6-(4-(tert-butyl)phenoxy)pyridin-3-yl)glycinate (3.28 mmol, 1.0 g, 90% yield).

C₁₈H₂₂N₂O₃; Mw=314.39 g.mol⁻¹; ¹H NMR (400 MHz, CDCl₃) δ 7.64 (dd, J=3.2, 0.6 Hz, 1H), 7.38-7.33 (m, 2H), 7.02 (dd, J=8.8, 3.1 Hz, 1H), 7.00-6.96 (m, 2H), 6.79 (dd, J=8.7, 0.6 Hz, 1H), 3.90 (s, 2H), 3.79 (s, 3H), 1.31 (s, 9H).

### Step 2: 2-((6-(4-(tert-Butyl)phenoxy)pyridin-3-yl)amino)acetohydrazide

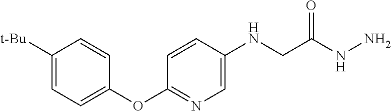 [SMILES: CC(C)(C)c1ccc(Oc2ccc(NCC(=O)NN)cn2)cc1]

To a solution of methyl (6-(4-(tert-butyl)phenoxy)pyridin-3-yl)glycinate (1.0 equiv, 1.72 mmol, 540 mg) in EtOH (0.1 M, 18 mL), hydrazine monohydrate (1.6 equiv, 2.78 mmol, 0.21 mL) was added. The colorless solution was stirred 5 h at 80° C. Volatiles were removed and the crude product was purified by combi flash column chromatography using CH₂Cl₂/MeOH as the eluent, to afford 2-((6-(4-(tert-butyl)phenoxy)pyridin-3-yl)amino)acetohydrazide (1.27 mmol, 400 mg, 74% yield).

C₁₇H₂₂N₄O₂; Mw=314.39 g.mol⁻¹; ¹H NMR (400 MHz, CDCl₃) δ 7.65 (d, J=3.1 Hz, 1H), 7.38-7.33 (m, 2H), 6.99 (m, 3H), 6.79 (d, J=8.8 Hz, 1H), 3.85 (s, 2H), 1.31 (s, 9H).

### Example 9

### 5-(((6-(4-(tert-Butyl)phenoxy)pyridin-3-yl)amino)methyl)-1,3,4-oxadiazol-2-amine

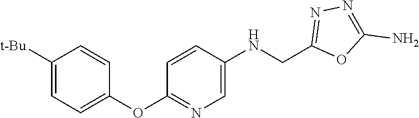 [SMILES: CC(C)(C)c1ccc(Oc2ccc(NCc3nnc(N)o3)cn2)cc1]

2-((6-(4-(tert-Butyl)phenoxy)pyridin-3-yl)amino)acetohydrazide (1.0 equiv, 80 μmol, 25 mg), BrCN (1.2 equiv, 95 μmol, 10 mg), and NaHCO₃ (1.2 equiv, 95 μmol, 8 mg) were added to a 5:2 mixture of dioxane and water (0.05 M, 1.4 mL) in a sealed microwave vial. The yellow suspension was sonicated for 5 h. The reaction mixture was poured into a mixture of EtOAc and sat. aq. NaHCO₃. The layers were separated and the organic layer was washed with brine, dried over Na₂SO₄ or MgSO₄, filtered-off and concentrated under reduced pressure. The crude product was purified by combi flash column chromatography using CH₂Cl₂/MeOH as the eluent, to afford 5-(((6-(4-(tert-butyl)phenoxy)pyridin-3-yl)amino)methyl)-1,3,4-oxadiazol-2-amine (35 μmol, 12 mg, 43% yield).

C₁₈H₂₁N₅O₂; Mw=339.40 g.mol⁻¹; ¹H NMR (400 MHz, DMSO-d₆) δ 7.60 (d, J=3.0 Hz, 1H), 7.37-7.32 (m, 2H), 7.18 (dd, J=8.8, 3.1 Hz, 1H), 6.96 (s, 2H), 6.91-6.86 (m, 2H), 6.82 (d, J=8.7 Hz, 1H), 6.29 (t, J=6.3 Hz, 1H), 4.32 (d, J=6.3 Hz, 2H), 1.27 (s, 9H).

### Example 10

### N-((1H-Imidazol-4-yl)methyl)-6-(4-(tert-butyl)phenoxy)pyridin-3-amine

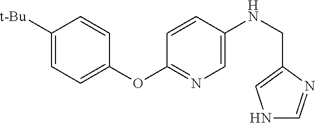 [SMILES: CC(C)(C)c1ccc(Oc2ccc(NCc3c[nH]cn3)cn2)cc1]

Following the General procedure C, N-((1H-imidazol-4-yl)methyl)-6-(4-(tert-butyl)phenoxy)pyridin-3-amine was obtained in 32% yield (0.12 mmol, 37 mg) from the HCl salt of 6-(4-(tert-butyl)phenoxy)pyridin-3-amine (0.36 mmol, 100 mg).

C₁₉H₂₂N₄O; Mw=322.41 g.mol⁻¹; ¹H NMR (400 MHz, CDCl₃) δ 7.69 (d, J=3.0 Hz, 1H), 7.62 (s, 1H), 7.37-7.32 (m, 2H), 7.07 (dd, J=8.7, 3.0 Hz, 1H), 6.99-6.94 (m, 3H), 6.77 (d, J=8.7 Hz, 1H), 4.29 (s, 2H), 1.31 (s, 9H).

### Example 11

### 6-((4′-Fluoro-[1,1′-biphenyl]-4-yl)oxy)-4-methyl-N-(pyridin-3-ylmethyl)pyridin-3-amine

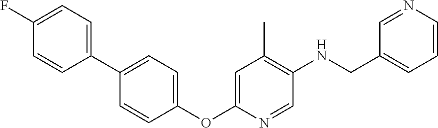 [SMILES: Cc1cc(Oc2ccc(-c3ccc(F)cc3)cc2)ncc1NCc1cccnc1]

Following the General procedure F, 6-((4′-fluoro-[1,1′-biphenyl]-4-yl)oxy)-4-methyl-N-(pyridin-3-ylmethyl)pyridin-3-amine was obtained in 45% yield (0.15 mmol, 0.047 g) from 6-((4′-fluoro-[1,1′-biphenyl]-4-yl)oxy)-4-methylpyridin-3-amine (0.340 mmol, 0.100 mg). C₂₄H₂₀FN₃O; Mw=385.43 g.mol⁻¹; ¹H NMR (400 MHz, CDCl₃) δ 8.58 (s, 1H), 8.49 (d, J=4.1 Hz, 1H), 7.64 (d, J=7.7 Hz, 1H), 7.46-7.39 (m, 4H), 7.22 (dd, J=7.8, 4.9 Hz, 2H), 7.07-6.99 (m, 4H), 6.70 (s, 1H), 4.33 (s, 2H), 3.58 (s, 1H), 2.15 (s, 3H).

The starting materials were prepared as followed:

### Step 1: 2-(4-Bromophenoxy)-4-methyl-5-nitropyridine

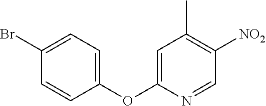 [SMILES: Cc1cc(Oc2ccc(Br)cc2)ncc1[N+](=O)[O-]]

Following the General procedure A, 2-(4-bromophenoxy)-4-methyl-5-nitropyridine was obtained in 40% yield (11 mmol, 3.50 g) from 4-bromophenol (31.9 mmol, 5.51 g) and 2-chloro-4-methyl-5-nitropyridine (29 mmol, 5.00 g).

C₁₂H₉BrN₂O₃; Mw=309.12 g.mol⁻¹; ¹H NMR (400 MHz, CDCl₃) δ 8.86 (s, 1H), 7.55 (d, J=8.9 Hz, 2H), 7.04 (d, J=8.9 Hz, 2H), 6.86 (d, J=1.0 Hz, 1H), 2.68 (app d, J=1.0 Hz, 3H).

### Step 2: 2-((4′-Fluoro-[1,1′-biphenyl]-4-yl)oxy)-4-methyl-5-nitropyridine

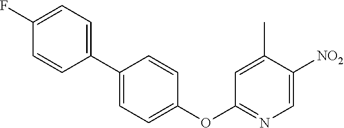 [SMILES: Cc1cc(Oc2ccc(-c3ccc(F)cc3)cc2)ncc1[N+](=O)[O-]]

Following the General procedure E, 2-((4′-fluoro-[1,1′-biphenyl]-4-yl)oxy)-4-methyl-5-nitropyridine was obtained in 82% yield (1.13 mmol, 0.30 g) from 2-(4-bromophenoxy)-4-methyl-5-nitropyridine (1.13 mmol, 0.350 mg) and (4-fluorophenyl)boronic acid (1.70 mmol, 0.238 mg).

C₁₈H₁₃FN₂O₃; Mw=324.31 g.mol⁻¹; ¹H NMR (400 MHz, CDCl₃) δ 8.91 (s, 1H), 7.63-7.58 (m, 2H), 7.57-7.52 (m, 2H), 7.23-7.19 (m, 2H), 7.17-7.11 (m, 2H), 6.89 (d, J=0.9 Hz, 1H), 2.69 (s, 3H).

### Step 3: 6-((4′-Fluoro-[1,1′-biphenyl]-4-yl)oxy)-4-methylpyridin-3-amine

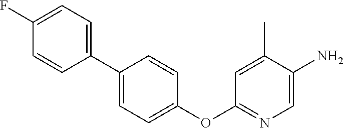 [SMILES: Cc1cc(Oc2ccc(-c3ccc(F)cc3)cc2)ncc1N]

Following the General procedure B, 6-((4′-fluoro-[1,1′-biphenyl]-4-yl)oxy)-4-methylpyridin-3-amine was obtained in 86% yield (476 mmol, 0.140 g) from 24(4′-fluoro-[1,1′-biphenyl]-4-yl)oxy)-4-methyl-5-nitropyridine (555 mmol, 0.180 g).

C₁₈H₁₅FN₂O; Mw=294.33 g.mol⁻¹; ¹H NMR (400 MHz, CDCl₃) δ 7.69 (s, 1H), 7.54-7.46 (m, 4H), 7.14-7.06 (m, 4H), 6.72 (s, 1H), 2.20 (s, 3H), 2.07 (s, 2H).

### Example 12

### 64(4′-Fluoro-[1,1′-biphenyl]-4-yl)oxy)-2-methyl-N-(pyridin-3-ylmethyl)pyridin-3-amine

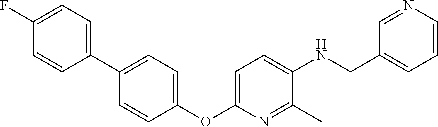 [SMILES: Cc1nc(Oc2ccc(-c3ccc(F)cc3)cc2)ccc1NCc1cccnc1]

Following the General procedure F, 6-((4′-fluoro-[1,1′-biphenyl]-4-yl)oxy)-2-methyl-N-(pyridin-3-ylmethyl)pyridin-3-amine was obtained in 32% yield (0.088 mmol, 34 mg) from 6-((4′-fluoro-[1,1′-biphenyl]-4-yl)oxy)-2-methylpyridin-3-amine (0.27 mmol, 80 mg).

C₂₄H₂₀FN₃O; Mw=385.44 g mol⁻¹; ¹H NMR (400 MHz, DMSO-d₆) δ 8.63-8.57 (m, 1H), 8.44 (dd, J=4.8, 1.7 Hz, 1H), 7.77 (dt, J=7.8, 1.9 Hz, 1H), 7.71-7.63 (m, 2H), 7.62-7.54 (m, 2H), 7.35 (ddd, J=7.8, 4.8, 0.8 Hz, 1H), 7.31-7.21 (m, 2H), 7.01-6.94 (m, 2H), 6.91 (d, J=8.6 Hz, 1H), 6.68 (d, J=8.5 Hz, 1H), 5.85 (t, J=6.1 Hz, 1H), 4.39 (d, J=6.1 Hz, 2H), 2.30 (s, 3H).

The starting materials were prepared as followed:

### Step 1: 6-(4-Bromophenoxy)-2-methyl-3-nitropyridine

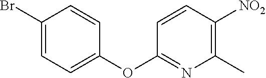 [SMILES: Cc1nc(Oc2ccc(Br)cc2)ccc1[N+](=O)[O-]]

Following the General procedure A, 6-(4-bromophenoxy)-2-methyl-3-nitropyridine was obtained in 93% yield (26.9 mmol, 8.31 g) from 6-chloro-2-methyl-3-nitropyridine (29 mmol, 5.00 g) and 4-bromophenol (32 mmol, 5.50 g).

C₁₂H₉BrN₂O₃; Mw=309.12 g mol⁻¹; ^{l}NMR (400 MHz, CDCl₃) δ 8.38 (d, J=8.9 Hz, 1H), 7.72-7.42 (m, 2H), 7.15-6.99 (m, 2H), 6.84 (d, J=8.9 Hz, 1H), 2.73 (s, 3H).

### Step 2: 64(4′-Fluoro-[1,1′-biphenyl]-4-yl)oxy)-2-methyl-3-nitropyridine

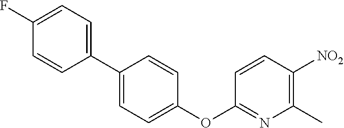 [SMILES: Cc1nc(Oc2ccc(-c3ccc(F)cc3)cc2)ccc1[N+](=O)[O-]]

Following the General procedure E, a mixture of (4-fluorophenyl)boronic acid (4.85 mmol, 679 mg), 6-(4-bromophenoxy)-2-methyl-3-nitropyridine (3.23 mmol, 1.00 g), K₂CO₃ (8.09 mmol, 1.12 g), and Pd(PPh₃)₄ (0.162 mmol, 187 mg, 5% mol) in 4:1 mixture of dioxane/H₂O (0.1 M) was converted to 6-((4′-fluoro-[1,1′-biphenyl]-4-yl)oxy)-2-methyl-3-nitropyridine in 90% yield (2.94 mmol, 900 mg).

C₁₈H₁₃FN₂O₃; Mw=324.31 g.mol⁻¹; ¹H NMR (400 MHz, CDCl₃) δ 8.38 (d, J=8.9 Hz, 1H), 7.62-7.58 (m, 2H), 7.58-7.53 (m, 2H), 7.25-7.21 (m, 2H), 7.19-7.11 (m, 2H), 6.84 (dd, J=8.9, 0.7 Hz, 1H), 2.77 (s, 3H).

### Step 3: 64((4′-Fluoro-[1,1′-biphenyl]-4-yl)oxy)-2-methylpyridin-3-amine

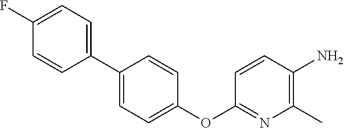 [SMILES: Cc1nc(Oc2ccc(-c3ccc(F)cc3)cc2)ccc1N]

Following the General procedure B, 6-((4′-fluoro-[1,1′-biphenyl]-4-yl)oxy)-2-methylpyridin-3-amine was obtained in 72% yield (1.11 mmol, 330 mg) from 6-((4′-fluoro-[1,1′-biphenyl]-4-yl)oxy)-2-methyl-3-nitropyridine (1.54 mmol, 500 mg).

C₁₈H₁₅FN₂O; Mw=294.33 g.mol⁻¹; ¹H NMR (400 MHz, DMSO-d₆) δ 7.71-7.63 (m, 2H), 7.63-7.56 (m, 2H), 7.32-7.22 (m, 2H), 7.08 (d, J=8.3 Hz, 1H), 7.02-6.96 (m, 2H), 6.67 (d, J=8.3 Hz, 1H), 4.90 (s, 2H), 2.18 (s, 3H).

### Example 13

### 4-Methyl-N-(pyridin-3-ylmethyl)-6-(4-(thiazol-5-yl)phenoxy)pyridin-3-amine

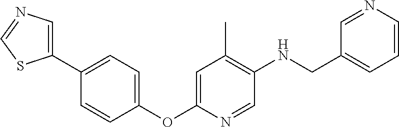 [SMILES: Cc1cc(Oc2ccc(-c3cncs3)cc2)ncc1NCc1cccnc1]

Following the General procedure F, 4-methyl-N-(pyridin-3-ylmethyl)-6-(4-(thiazol-5-yl)phenoxy)pyridin-3-amine was obtained in 28% yield (80 mmol, 0.030 g) from 4-methyl-6-(4-(thiazol-5-yl)phenoxy)pyridin-3-amine (0.28 mmol, 0.080 g).

C₂₁H₁₈N₄OS; Mw=374.46 g.mol⁻¹; ¹H NMR (400 MHz, CDCl₃) δ 8.72 (s, 1H), 8.66 (s, 1H), 8.56 (s, 1H), 8.00 (s, 1H), 7.71 (dt, J=7.9, 1.9 Hz, 1H), 7.57-7.49 (m, 3H), 7.30 (dd, J=7.8, 4.8 Hz, 1H), 7.12-7.06 (m, 2H), 6.77 (s, 1H), 4.40 (d, J=3.2 Hz, 2H), 3.68 (s, 1H), 2.22 (s, 3H).

The starting material was prepared as follows:

### Step 1: 2-(4-Bromophenoxy)-4-methyl-5-nitropyridine

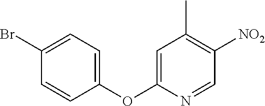 [SMILES: Cc1cc(Oc2ccc(Br)cc2)ncc1[N+](=O)[O-]]

Following the General procedure A, 2-(4-bromophenoxy)-4-methyl-5-nitropyridine was obtained in 40% yield (11 mmol, 3.50 g) from 4-bromophenol (31.9 mmol, 5.51 g) and 2-chloro-4-methyl-5-nitropyridine (29 mmol, 5.00 g).

C₁₂H₉BrN₂O₃; Mw=309.12 g.mol⁻¹; ¹H NMR (400 MHz, CDCl₃) δ 8.86 (s, 1H), 7.55 (d, J=8.9 Hz, 2H), 7.04 (d, J=8.9 Hz, 2H), 6.86 (d, J=1.0 Hz, 1H), 2.68 (app d, J=1.0 Hz, 3H).

### Step 2: 5-(4-((4-Methyl-5-nitropyridin-2-yl)oxy)phenyl)thiazole

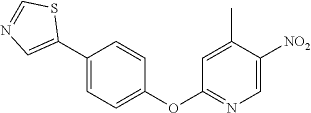 [SMILES: Cc1cc(Oc2ccc(-c3cncs3)cc2)ncc1[N+](=O)[O-]]

Following the General procedure E, 5-(4-((4-methyl-5-nitropyridin-2-yl)oxy)phenyl)thiazole was obtained in 40% yield (1.02 mmol, 0.320 g) from 5-(4,4,5,5-tetramethyl-1,3-dioxolan-2-yl)thiazole (3.78 mmol, 0.799 mg) and 2-(4-bromophenoxy)-4-methyl-5-nitropyridine (2.52 mmol, 0.780 g).

C₁₅H₁₁N₃O₃S; Mw=313.33 g.mol⁻¹; ¹H NMR (400 MHz, CDCl₃) δ 8.89 (s, 1H), 8.78 (s, 1H), 8.07 (s, 1H), 7.65 (d, J=8.7 Hz, 2H), 7.21 (d, J=8.7 Hz, 2H), 6.90 (s, 1H), 2.69 (s, 3H).

### Step 3: 4-Methyl-6-(4-(thiazol-5-yl)phenoxy)pyridin-3-amine

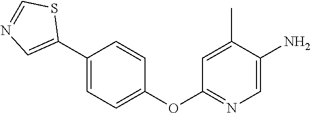 [SMILES: Cc1cc(Oc2ccc(-c3cncs3)cc2)ncc1N]

Following the General procedure B, 4-methyl-6-(4-(thiazol-5-yl)phenoxy)pyridin-3-amine was obtained in 33% yield (0.424 mmol, 0.120 g) from 5-(4-((4-methyl-5-nitropyridin-2-yl)oxy)phenyl)thiazole (1.02 mmol, 0.320 g).

C₁₅H₁₃N₃OS; Mw=283.35 g.mol⁻¹; ^{l}H NMR (400 MHz, CDCl₃) δ 8.72 (s, 1H), 8.00 (s, 1H), 7.67 (s, 1H), 7.54 (d, J=8.7 Hz, 2H), 7.09 (d, J=8.7 Hz, 2H), 6.73 (s, 1H), 2.21 (s, 3H), 2.04 (s, 2H).

### Example 14

### 2-Methyl-N-(pyridin-3-ylmethyl)-6-(4-(thiazol-5-yl)phenoxy)pyridin-3-amine

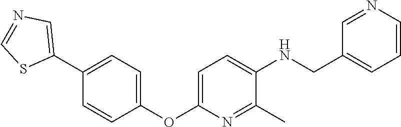 [SMILES: Cc1nc(Oc2ccc(-c3cncs3)cc2)ccc1NCc1cccnc1]

Following the General procedure F, 2-methyl-N-(pyridin-3-ylmethyl)-6-(4-(thiazol-5-yl)phenoxy)pyridin-3-amine was obtained in 61% yield (0.15 mmol, 56 mg) from 2-methyl-6-(4-(thiazol-5-yl)phenoxy)pyridin-3-amine (0.25 mmol, 70 mg).

C₂₁H₁₈N₄OS; Mw=374.46 g mol⁻¹; ¹H NMR (400 MHz, DMSO-d₆) δ 9.03 (d, J=0.7 Hz, 1H), 8.67-8.52 (m, 1H), 8.44 (dd, J=4.8, 1.7 Hz, 1H), 8.22 (d, J=0.7 Hz, 1H), 7.77 (dt, J=7.9, 2.0 Hz, 1H), 7.63 (d, J=8.7 Hz, 2H), 7.35 (ddd, J=7.9, 4.8, 0.9 Hz, 1H), 7.00-6.95 (m, 2H), 6.91 (d, J=8.6 Hz, 1H), 6.69 (d, J=8.6 Hz, 1H), 5.87 (t, J=6.1 Hz, 1H), 4.39 (d, J=6.1 Hz, 2H), 2.30 (s, 3H).

The starting material was prepared as follows:

### Step 1: 5-(4-((6-Methyl-5-nitropyridin-2-yl)oxy)phenyl)thiazole

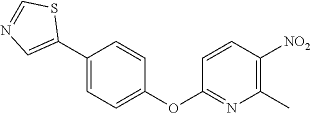 [SMILES: Cc1nc(Oc2ccc(-c3cncs3)cc2)ccc1[N+](=O)[O-]]

Following the General procedure D, a mixture of 5-(4,4,5,5-tetramethyl-1,3,2-dioxaborolan-2-yl)thiazole (4.89 mmol, 1.03 g), 6-(4-bromophenoxy)-2-methyl-3-nitropyridine (3.26 mmol, 1.01 g; expl. 12, Step 1), Na₂CO₃ (8.15 mmol, 864 mg), and Pd(PPh₃)₄ (0.163 mmol, 188 mg, 5% mol) in a 4:1 mixture of dioxane/H₂O (0.06 M) was converted to 5-(4-((6-methyl-5-nitropyridin-2-yl)oxy)phenyl)thiazole in 20% yield (0.67 mmol, 209 mg).

C₁₅H₁₁N₃O₃S; Mw=313.33 g.mol⁻¹; ¹H NMR (400 MHz, DMSO-d₆) δ 9.10 (app d, J=0.7 Hz, 1H), 8.52 (d, J=8.9 Hz, 1H), 8.33 (s, 1H), 7.91-7.71 (m, 2H), 7.40-7.21 (m, 2H), 7.11 (d, J=8.9 Hz, 1H), 2.60 (s, 3H).

### Step 2: 2-Methyl-6-(4-(thiazol-5-yl)phenoxy)pyridin-3-amine

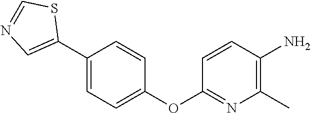 [SMILES: Cc1nc(Oc2ccc(-c3cncs3)cc2)ccc1N]

Following the General procedure B, 2-methyl-6-(4-(thiazol-5-yl)phenoxy)pyridin-3-amine was obtained in 64% yield (0.42 mmol, 120 mg) from 5-(4-((6-methyl-5-nitropyridin-2-yl)oxy)phenyl)thiazole (0.66 mmol, 206 mg).

C₁₅H₁₃N₃OS; Mw=283.35 g.mol⁻¹; ^{l}NMR (400 MHz, DMSO-d₆) δ 9.04 (d, J=0.7 Hz, 1H), 8.22 (d, J=0.7 Hz, 1H), 7.63 (d, J=8.8 Hz, 2H), 7.08 (d, J=8.3 Hz, 1H), 6.99 (d, J=8.7 Hz, 2H), 6.68 (d, J=8.4 Hz, 1H), 4.92 (s, 2H), 2.17 (s, 3H).

### Example 15

### 64(2,2′-Dimethyl-[1,1′-biphenyl]-4-yl)oxy)-2-methyl-N-(pyridin-3-ylmethyl)pyridin-3-amine

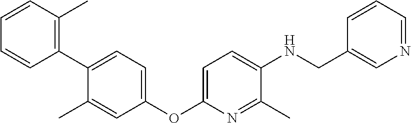 [SMILES: Cc1ccccc1-c1ccc(Oc2ccc(NCc3cccnc3)c(C)n2)cc1C]

Following the General procedure F, 6-((2,2′-dimethyl-[1,1′-biphenyl]-4-yl)oxy)-2-methyl-N-(pyridin-3-ylmethyl)pyridin-3-amine was obtained in 30% yield (0.09 mmol, 35 mg) from 6-((2,2′-dimethyl-[1,1′-biphenyl]-4-yl)oxy)-2-methylpyridin-3-amine (0.30 mmol, 90 mg).

C₂₆H₂₅N₃O; Mw=395.51 g mol⁻¹; ¹H NMR (400 MHz, CDCl₃) δ 8.69 (s, 1H), 8.59 (d, J=4.3 Hz, 1H), 7.82 (d, J=7.8 Hz, 1H), 7.40 (dd, J=7.7, 5.0 Hz, 1H), 7.25-7.18 (m, 3H), 7.10 (d, J=6.8 Hz, 1H), 7.03 (d, J=8.4 Hz, 1H), 6.93 (d, J=2.5 Hz, 1H), 6.89-6.82 (m, 2H), 6.64 (d, J=8.4 Hz, 1H), 4.44 (s, 2H), 3.88 (s, 1H), 2.43 (s, 3H), 2.07 (s, 3H), 2.01 (s, 3H).

The starting material was prepared as follows:

### Step 1: 6-(4-Bromo-3-methylphenoxy)-2-methyl-3-nitropyridine

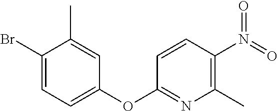 [SMILES: Cc1cc(Oc2ccc([N+](=O)[O-])c(C)n2)ccc1Br]

Following the General procedure A, 6-(4-bromo-3-methylphenoxy)-2-methyl-3-nitropyridine was obtained in 94% yield (5.46 mmol, 1.76 g) from 6-chloro-2-methyl-3-nitropyridine (5.79 mmol, 1.00 g) and 4-bromo-3-methylphenol (6.37 mmol, 1.19 g).

C₁₃H₁₁BrN₂O₃; Mw=323.15 g mol⁻¹; ¹H NMR (400 MHz, CDCl₃) δ 8.36 (d, J=8.9 Hz, 1H), 7.56 (d, J=8.6 Hz, 1H), 7.05 (d, J=2.6 Hz, 1H), 6.88 (dd, J=8.6, 2.6 Hz, 1H), 6.80 (d, J=8.9 Hz, 1H), 2.74 (s, 3H), 2.42 (s, 3H).

### Step 2: 6-((2,2′-Dimethyl-[1,1′-biphenyl]-4-yl)oxy)-2-methyl-3-nitropyridine

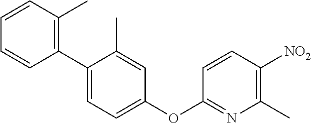 [SMILES: Cc1ccccc1-c1ccc(Oc2ccc([N+](=O)[O-])c(C)n2)cc1C]

Following the General procedure D, a mixture of o-tolylboronic acid (3.71 mmol, 505 mg), 6-(4-bromo-3-methylphenoxy)-2-methyl-3-nitropyridine (2.48 mmol, 800 mg), Na₂CO₃ (4.95 mmol, 525 mg), and Pd(PPh₃)₄ (0.124 mmol, 143 mg, 5% mol) in a 3:1 mixture of DME/H₂O (0.43 M) was converted to 64(2,2′-dimethyl-[1,1′-biphenyl]-4-yl)oxy)-2-methyl-3-nitropyridine in 90% yield (2.24 mmol, 750 mg).

C₂₀H₁₈N₂O₃; Mw=334.38 g.mol⁻¹; ¹H NMR (400 MHz, CDCl₃) δ 8.38 (d, J=8.9 Hz, 1H), 7.33-7.21 (m, 3H), 7.19-7.09 (m, 2H), 7.07 (d, J=2.5 Hz, 1H), 7.02 (dd, J=8.2, 2.5 Hz, 1H), 6.81 (d, J=8.9 Hz, 1H), 2.81 (s, 3H), 2.10 (s, 3H), 2.08 (s, 3H).

### Step 3: 6-((2,2′-Dimethyl-[1,1′-biphenyl]-4-yl)oxy)-2-methylpyridin-3-amine

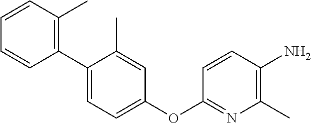 [SMILES: Cc1ccccc1-c1ccc(Oc2ccc(N)c(C)n2)cc1C]

Following the General procedure B, 6-((2,2′-dimethyl-[1,1′-biphenyl]-4-yl)oxy)-2-methylpyridin-3-amine was obtained in 80% yield (0.60 mmol, 183 mg) from 64(2,2′-dimethyl-[1,1′-biphenyl]-4-yl)oxy)-2-methyl-3-nitropyridine (0.75 mmol, 250 mg).

C₂₀H₂₀N₂O; Mw=304.39 g.mol⁻¹; ¹H NMR (400 MHz, methanol-d₄) δ 7.29-7.16 (m, 4H), 7.06-7.03 (m, 1H), 7.01 (d, J=8.3 Hz, 1H), 6.88 (d, J=2.6 Hz, 1H), 6.80 (dd, J=8.3, 2.6 Hz, 1H), 6.63 (dd, J=8.5, 0.7 Hz, 1H), 2.33 (s, 3H), 2.05 (s, 3H), 1.99 (s, 3H).

### Example 16

### 2-Methyl-N-(pyridin-3-ylmethyl)-6-(4-(thiazol-2-yl)phenoxy)pyridin-3-amine

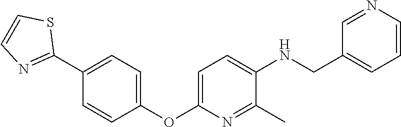 [SMILES: Cc1nc(Oc2ccc(-c3nccs3)cc2)ccc1NCc1cccnc1]

Following the General procedure F, 2-methyl-N-(pyridin-3-ylmethyl)-6-(4-(thiazol-2-yl)phenoxy)pyridin-3-amine was obtained in 35% yield (0.16 mmol, 0.060 g) from 2-methyl-6-(4-(thiazol-2-yl)phenoxy)pyridin-3-amine (452 mmol, 0.128 g).

C₂₁H₁₈N₄OS; Mw=374.46 g.mol⁻¹; ¹H NMR (400 MHz, CDCl₃) δ 8.70-8.67 (m, 1H), 8.60 (dd, J=4.8, 1.3 Hz, 1H), 7.96-7.90 (m, 2H), 7.85 (d, J=3.3 Hz, 1H), 7.76-7.69 (m, 1H), 7.34 (dd, J=7.6, 4.9 Hz, 1H), 7.30 (d, J=3.3 Hz, 1H), 7.11-7.05 (m, 2H), 6.94 (d, J=8.6 Hz, 1H), 6.71 (d, J=8.6 Hz, 1H), 4.43 (d, J=2.7 Hz, 2H), 3.87 (s, 1H), 2.41 (s, 3H).

The starting material was prepared as follows:

### Step 1: 2-(4-((6-Methyl-5-nitropyridin-2-yl)oxy)phenyl)thiazole

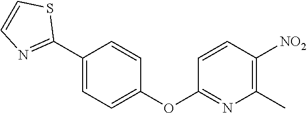 [SMILES: Cc1nc(Oc2ccc(-c3nccs3)cc2)ccc1[N+](=O)[O-]]

Following the General procedure A, 2-(4-((6-methyl-5-nitropyridin-2-yl)oxy)phenyl)thiazole was obtained in 51% yield (1.44 mmol, 0.450 g) from 4-(2-thiazolyl)phenol (2.82 mmol, 500 mg) and 6-chloro-2-methyl-3-nitropyridine (3.10 mmol, 536 mg).

C₁₅H₁₁N₃O₃S; Mw=313.33 g.mol⁻¹; ¹H NMR (400 MHz, CDCl₃) δ 8.39 (d, J=8.9 Hz, 1H), 8.04 (d, J=8.5 Hz, 2H), 7.88 (s, 1H), 7.36 (s, 1H), 7.25 (d, J=8.2 Hz, 2H), 6.87 (d, J=8.9 Hz, 1H), 2.74 (s, 3H).

### Step 2: 2-Methyl-6-(4-(thiazol-2-yl)phenoxy)pyridin-3-amine

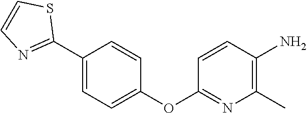 [SMILES: Cc1nc(Oc2ccc(-c3nccs3)cc2)ccc1N]

Following the General procedure B, 2-methyl-6-(4-(thiazol-2-yl)phenoxy)pyridin-3-amine was obtained in 68% yield (0.434 mmol, 123 mg) from 2-(4-((6-methyl-5-nitropyridin-2-yl)oxy)phenyl)thiazole (0.638 mmol, 200 mg).

C₁₅H₁₃N₃OS; Mw=283.35 g.mol⁻¹; ¹H NMR (400 MHz, CDCl₃) δ 7.85 (d, J=8.8 Hz, 2H), 7.76 (d, J=3.3 Hz, 1H), 7.22 (d, J=3.3 Hz, 1H), 7.01 (d, J=8.8 Hz, 2H), 6.97 (d, J=8.4 Hz, 1H), 6.59 (d, J=8.4 Hz, 1H), 2.30 (s, 3H).

### Example 17

### 2-Methyl-6-((6-phenylpyridin-3-yl)oxy)-N-(pyridin-3-ylmethyl)pyridin-3-amine

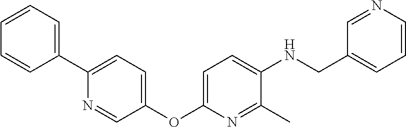 [SMILES: Cc1nc(Oc2ccc(-c3ccccc3)nc2)ccc1NCc1cccnc1]

Following the General procedure F, 2-methyl-6-((6-phenylpyridin-3-yl)oxy)-N-(pyridin-3-ylmethyl)pyridin-3-amine was obtained in 71% yield (0.20 mmol, 75 mg) from 2-methyl-6-((6-phenylpyridin-3-yl)oxy)pyridin-3-amine (0.29 mmol, 80 mg).

C₂₃H₂₀N₄O; Mw=368.44 g mol⁻¹; ¹H NMR (400 MHz, DMSO-d₆) δ 8.61 (d, J=2.2 Hz, 1H), 8.44 (dd, J=4.8, 1.6 Hz, 1H), 8.39 (d, J=2.8 Hz, 1H), 8.10-8.01 (m, 2H), 7.93 (d, J=8.8 Hz, 1H), 7.77 (dd, J=7.9, 2.0 Hz, 1H), 7.52-7.44 (m, 3H), 7.43-7.40 (m, 1H), 7.39-7.32 (m, 1H), 6.94 (d, J=8.5 Hz, 1H), 6.76 (d, J=8.5 Hz, 1H), 5.88 (t, J=6.2 Hz, 1H), 4.40 (d, J=6.1 Hz, 2H), 2.29 (s, 3H).

The starting material was prepared as follows:

### Step 1: 6-Phenylpyridin-3-ol

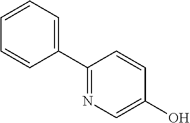 [SMILES: Oc1ccc(-c2ccccc2)nc1]

Following the General procedure D, a mixture of phenylboronic acid (11.49 mmol, 1.40 g), 6-bromopyridin-3-ol (5.75 mmol, 1.00 g), Na₂CO₃ (11.49 mmol, 1.22 g), and Pd(PPh₃)₄ (0.287 mmol, 332 mg) in a 3:1 mixture of DME/H₂O (0.32 M) was converted to 6-phenylpyridin-3-ol in 61% yield (3.50 mmol, 600 mg).

C₁₁H₉NO; Mw=171.20 g mol⁻¹; ¹H NMR (400 MHz, DMSO-d₆) δ 9.19 (s, 1H), 7.40-7.34 (m, 1H), 7.15-7.09 (m, 2H), 6.95 (dd, J=8.6, 0.7 Hz, 1H), 6.65-6.57 (m, 2H), 6.54-6.49 (m, 1H), 6.40 (dd, J=8.6, 2.9 Hz, 1H).

### Step 2: 2-Methyl-3-nitro-6-((6-phenylpyridin-3-yl)oxy)pyridine

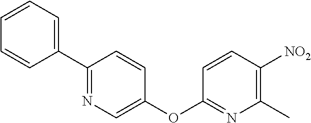 [SMILES: Cc1nc(Oc2ccc(-c3ccccc3)nc2)ccc1[N+](=O)[O-]]

Following the General procedure A, 2-methyl-3-nitro-6((6-phenylpyridin-3-yl)oxy)pyridine was obtained in 95% yield (1.22 mmol, 374 mg) from 6-chloro-2-methyl-3-nitropyridine (1.27 mmol, 220 mg).

C₁₇H₁₃N₃O₃; Mw=307.31 g.mol⁻¹; ¹H NMR (400 MHz, CDCl₃) δ 8.60 (dd, J=2.8, 0.7 Hz, 1H), 8.42 (d, J=8.9 Hz, 1H), 8.09-7.93 (m, 2H), 7.82 (dd, J=8.7, 0.7 Hz, 1H), 7.62 (dd, J=8.7, 2.8 Hz, 1H), 7.56-7.40 (m, 3H), 6.96 (d, J=8.9 Hz, 1H), 2.73 (s, 3H).

### Step 3: 2-Methyl-6-((6-phenylpyridin-3-yl)oxy)pyridin-3-amine

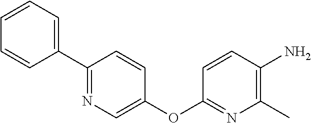 [SMILES: Cc1nc(Oc2ccc(-c3ccccc3)nc2)ccc1N]

Following the General procedure B, 2-methyl-6((6-phenylpyridin-3-yl)oxy)pyridin-3-amine was obtained in 89% yield (1.08 mmol, 300 mg) from 2-methyl-3-nitro-6-((6-phenylpyridin-3-yl)oxy)pyridine (1.22 mmol, 374 mg).

C₁₇H₁₅N₃O; Mw=277.33 g.mol⁻¹; ¹H NMR (400 MHz, DMSO-d₆) δ 8.40 (d, J=2.5 Hz, 1H), 8.15-7.98 (m, 2H), 7.94 (d, J=8.7 Hz, 1H), 7.55-7.45(m, 3H), 7.44-7.37 (m, 1H), 7.10 (d, J=8.4 Hz, 1H), 6.75 (d, J=8.4 Hz, 1H), 4.93 (s, 2H), 2.17 (s, 3H).

### Example 18

### 4-(4-(tert-Butyl)phenoxy)-3-fluoro-N-(pyridin-3-ylmethyl)aniline

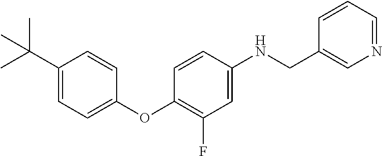 [SMILES: CC(C)(C)c1ccc(Oc2ccc(NCc3cccnc3)cc2F)cc1]

Following the General procedure H, 4-(4-(tert-butyl)phenoxy)-3-fluoro-N-(pyridin-3-ylmethyl)aniline was obtained in 40% yield (0.23 mmol, 80 mg) from 4-(4-(tert-butyl)phenoxy)-3-fluoroaniline (0.578 mmol, 150 mg; preparation see: WO 2013 093885).

C₂₂H₂₃FN₂O; Mw=350.44 g mol⁻¹; ¹H NMR (400 MHz, CDCl₃) δ 8.65-8.63 (m, 1H), 8.57-8.53 (m, 1H), 7.73-7.68 (m, 1H), 7.32-7.26 (m, 3H), 6.94 (t, J=8.9 Hz, 1H), 6.86-6.80 (m, 2H), 6.46-6.41 (m, 1H), 6.39-6.34 (m , 1H), 4.34 (s, 2H), 4.14 (s, 1H), 1.29 (s, 9H).

### Example 19

### 4-(4-Cyclohexylphenoxy)-3-fluoro-N-(pyridin-3-ylmethyl)aniline

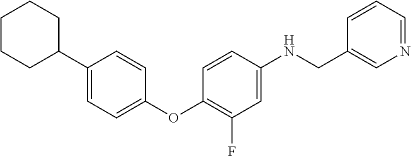 [SMILES: Fc1cc(NCc2cccnc2)ccc1Oc1ccc(C2CCCCC2)cc1]

Following the General procedure H, 4-(4-cyclohexylphenoxy)-3-fluoro-N-(pyridin-3-ylmethyl)aniline was obtained in 61% yield (0.21 mmol, 80 mg) from 4-(4-cyclohexylphenoxy)-3-fluoroaniline (0.35 mmol, 100 mg; preparation see: WO 2013 093885).

C₂₄H₂₅FN₂O; Mw=376.48 g mol⁻¹; ¹H NMR (400 MHz, CDCl₃) δ 8.64 (s, 1H), 8.58-8.54 (m, 1H), 7.74-7.69 (m, 1H), 7.33-7.28 (m, 1H), 7.12-7.07 (m, 2H), 6.93 (t, J=8.9 Hz, 1H), 6.86-6.79 (m, 2H), 6.47-6.41 (m, 1H), 6.38-6.34 (m, 1H), 4.35 (s, 2H), 4.10 (bs, 1H), 2.49-2.41(m, 1H), 1.88-1.78 (m, 4H), 1.77-1.70 (m, 2H), 1.41-1.32 (m, 4H).

### Example 20

### 6-([1,1r-Biphenyl]-4-yloxy)-N-(pyridin-3-ylmethyl)pyridin-3-amine

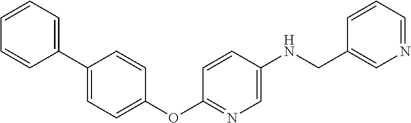 [SMILES: c1ccc(-c2ccc(Oc3ccc(NCc4cccnc4)cn3)cc2)cc1]

Following the General procedure H, 6-([1,1′-biphenyl]-4-yloxy)-N-(pyridin-3-ylmethyl)pyridin-3-amine was obtained in 25% yield (0.14 mmol, 50 mg) from 6-([1,1′-biphenyl]-4-yloxy)pyridin-3-amine (0.572 mmol, 150 mg).

C₂₃H₁₉N₃O; Mw=253.43 g mol⁻¹; ¹H NMR¹H NMR (400 MHz, CDCl₃) δ 8.64 (s, 1H), 8.55 (d, J=5.0 Hz, 1H), 7.73-7.66 (m, 2H), 7.60-7.52 (m, 4H), 7.47-7.38 (m, 2H), 7.35-7.27 (m, 2H), 7.14-7.10 (m, 2H), 7.06-7.01 (m, 1H), 6.85-6.81 (m, 1H), 4.36 (s, 2H), 4.02 (s, 1H).

The starting material was prepared as follows:

### Step 1: 2-([1,1′-Biphenyl]-4-yloxy)-5-nitropyridine

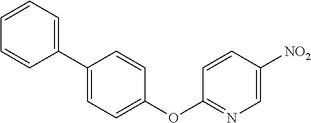 [SMILES: O=[N+]([O-])c1ccc(Oc2ccc(-c3ccccc3)cc2)nc1]

Following the General procedure A, 2-([1,1′-biphenyl]-4-yloxy)-5-nitropyridine was obtained in 99% yield without purification (4.65 mmol, 1.36 g) from [1,1′-biphenyl]-4-ol (4.70 mmol, 800 mg) and 2-chloro-5-nitropyridine (4.70 mmol, 745 mg).

C₁₇H₁₂N₂O₃; Mw=292.29 g.mol⁻¹; ¹H NMR (400 MHz, CDCl₃) δ 9.08 (d, J=2.8 Hz, 1H), 8.50 (dd, J=9.1, 2.8 Hz, 1H), 7.67 (d, J=8.8 Hz, 2H), 7.60 (dd, J=8.3, 1.2 Hz, 2H), 7.46 (dd, J=8.2, 6.8 Hz, 2H), 7.40-7.33 (m, 1H), 7.26-7.20 (m, 2H), 7.08 (dd, J=9.1, 0.5 Hz, 1 H).

### Step 2: 6-([1,1′-Biphenyl]-4-yloxy)pyridin-3-amine

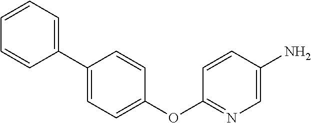 [SMILES: Nc1ccc(Oc2ccc(-c3ccccc3)cc2)nc1]

Following the General procedure B, 6-([1,1′-biphenyl]-4-yloxy)pyridin-3-amine was obtained in 97% yield (1.66 mmol, 434 mg) from 2-([1,1′-biphenyl]-4-yloxy)-5-nitropyridine (1.71 mmol, 500 mg).

C₁₇H₁₄N₂O; Mw=262.31 g.mol⁻¹; ¹H NMR (400 MHz, CDCl₃) δ 7.75 (dd, J=3.0, 0.6 Hz, 1H), 7.61-7.52 (m, 4 H), 7.47-7.39 (m, 2H), 7.36-7.29 (m, 1H), 7.17-7.06 (m, 3H), 6.82 (dd, J=8.6, 0.6 Hz, 1H).

### Example 21

### 6-((6-Phenylpyridin-3-yl)oxy)-N-(pyridin-3-ylmethyl)pyridin-3-amine

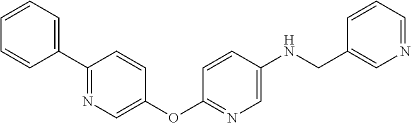 [SMILES: c1ccc(-c2ccc(Oc3ccc(NCc4cccnc4)cn3)cn2)cc1]

Following the General procedure H, 6-((6-phenylpyridin-3-yl)oxy)-N-(pyridin-3-ylmethyl)pyridin-3-amine was obtained in 49% yield (0.282 mmol, 100 mg) from 6-((6-phenylpyridin-3-yl)oxy)pyridin-3-amine (0.576 mmol, 152 mg).

C₂₂H₁₈N₄O; Mw=354.41 g mol⁻¹; ¹H NMR (400 MHz, CDCl₃) δ 8.66 (s, 1H), 8.59-8.54 (m, 1H), 8.52 (d, J=2.8 Hz, 1H), 7.97-7.92 (m, 2H), 7.79-7.74 (m, 1H), 7.72 (d, J=8.7 Hz, 1H), 7.62 (d, J=3.0 Hz, 1H), 7.54-7.50 (m, 1H), 7.49-7.42 (m, 2H), 7.42-7.33 (m, 2H), 7.09-7.04 (m, 1H), 6.89 (d, J=8.7 Hz, 1H), 4.40 (s, 2H), 4.06 (s, 1H).

The starting material was prepared as follows:

### Step 1: 5-Nitro-2-((6-phenylpyridin-3-yl)oxy)pyridine

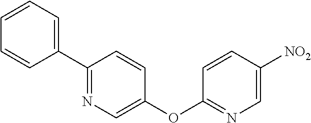 [SMILES: O=[N+]([O-])c1ccc(Oc2ccc(-c3ccccc3)nc2)nc1]

Following the General procedure A, 5-nitro-2-((6-phenylpyridin-3-yl)oxy)pyridine was obtained in 93% yield (2.63 mmol, 0.77 g) from 6-phenylpyridin-3-ol (2.92 mmol, 0.51 g) and 2-chloro-5-nitropyridine (2.84 mmol, 0.45 g).

C₁₆H₁₁N₃O₃; Mw=293.28 g.mol⁻¹; Solid; ¹H NMR (400 MHz, CDCl₃) δ 9.04 (d, J=2.7 Hz, 1H), 8.59 (d, J=2.6 Hz, 1H), 8.54 (dd, J=9.0, 2.8 Hz, 1H), 8.04-7.97 (m, 2H), 7.83 (d, J=8.6 Hz, 1H), 7.62 (dd, J=8.6, 2.8 Hz, 1H), 7.52-7.47 (m, 2H), 7.45 (dd, J=4.9, 3.6 Hz, 1H), 7.16 (d, J=9.0 Hz, 1H).

### Step 2: 6-((6-Phenylpyridin-3-yl)oxy)pyridin-3-amine

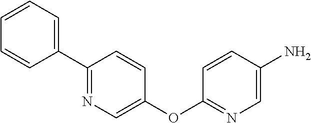 [SMILES: Nc1ccc(Oc2ccc(-c3ccccc3)nc2)nc1]

Following the General procedure B, 6-((6-phenylpyridin-3-yl)oxy)pyridin-3-amine was obtained in 93% yield (2.43 mmol, 0.64 g) from 5-nitro-2-((6-phenylpyridin-3-yl)oxy)pyridine (2.63 mmol, 0.77 g).

C₁₆H₁₃N₃O; Mw=263.30 g.mol⁻¹; Solid; ¹H NMR (400 MHz, CDCl₃) δ 8.53 (d, J=2.7 Hz, 1H), 7.96 (d, J=7.3 Hz, 2H), 7.71 (dd, J=7.9, 5.9 Hz, 2H), 7.51 (dd, J=8.6, 2.7 Hz, 1H), 7.46 (t, J=7.5 Hz, 2H), 7.39 (t, J=7.3 Hz, 1H), 7.13 (dd, J=8.6, 2.9 Hz, 1H), 6.86 (d, J=8.6 Hz, 1H), 3.63 (s, 2H).

### Example 22

### 6-([1,1′-Biphenyl]-4-yloxy)-4-methyl-N-(pyridin-3-ylmethyl)pyridin-3-amine

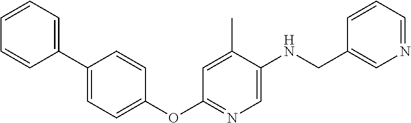 [SMILES: Cc1cc(Oc2ccc(-c3ccccc3)cc2)ncc1NCc1cccnc1]

Following the General procedure H, 6-([1,1′-biphenyl]-4-yloxy)-4-methyl-N-(pyridin-3-ylmethyl)pyridin-3-amine was obtained in 44% yield (0.24 mmol, 90 mg) from 6-([1,1′-biphenyl]-4-yloxy)-4-methylpyridin-3-amine (0.543 mmol, 150 mg).

C₂₄H₂₁N₃O; Mw=367.45 g mol⁻¹; ¹H NMR (400 MHz, CDCl₃) δ 8.65 (d, J=2.2 Hz, 1H), 8.57-8.53 (m, 1H), 7.73-7.69 (m, 1H), 7.58-7.51 (m, 5H), 7.45-7.38 (m, 2H), 7.35-7.27 (m, 2H), 7.15-7.09 (m, 2H), 6.79-6.75 (m, 1H), 4.40 (s, 2H), 3.66 (s, 1H), 2.22 (s, 3H).

The starting material was prepared as follows:

### Step 1: 6-([1,1′-Biphenyl]-4-yloxy)-4-methyl-3-nitropyridine

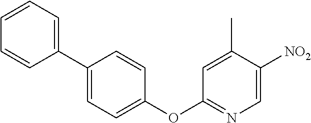 [SMILES: Cc1cc(Oc2ccc(-c3ccccc3)cc2)ncc1[N+](=O)[O-]]

Following the General procedure A, 6-([1,1′-biphenyl]-4-yloxy)-4-methyl-3-nitropyridine was obtained in 38% yield (2.3 mmol, 0.71 g) from [1,1′-biphenyl]-4-ol (5.9 mmol, 1.00 g) and 6-chloro-4-methyl-3-nitropyridine (5.8 mmol, 1.00 g).

C₁₈H₁₄N₂O₃; Mw=306.32 g.mol⁻¹; ¹H NMR (400 MHz, CDCl₃) δ 8.92 (s, 1H), 7.69-7.63 (m, 2H), 7.63-7.57 (m, 2H), 7.49-7.42 (m, 2H), 7.40-7.34 (m, 1H), 7.25-7.20 (m, 2H), 6.88 (d, J=0.9 Hz, 1H), 2.69 (d, J=0.8 Hz, 3H).

### Step 2: 6-([1,1′-Biphenyl]-4-yloxy)-4-methylpyridin-3-amine

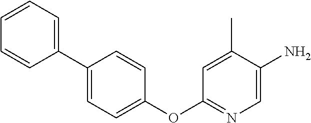 [SMILES: Cc1cc(Oc2ccc(-c3ccccc3)cc2)ncc1N]

Following the General procedure B, 6-([1,1′-biphenyl]-4-yloxy)-4-methylpyridin-3-amine was obtained in 74% yield (1.5 mmol, 410 mg) from 6-([1,1′-biphenyl]-4-yloxy)-4-methyl-3-nitropyridine (2.0 mmol, 600 mg).

C₁₈H₁₆N₂O; Mw=276.34 g.mol⁻¹; ¹HNMR (400 MHz, CDCl₃) δ 7.69 (s, 1H), 7.61-7.52 (m, 4H), 7.42 (t, J=7.6 Hz, 2H), 7.33 (dt, J=9.2, 4.3 Hz, 1H), 7.12 (d, J=8.7 Hz, 2H), 6.72 (s, 1H), 3.03 (s, 2H), 2.21 (d, J=0.5 Hz, 3H).

### Example 23

### 6-([1,1′-Biphenyl]-4-yloxy)-2-methyl-N-(pyridin-3-ylmethyl)pyridin-3-amine

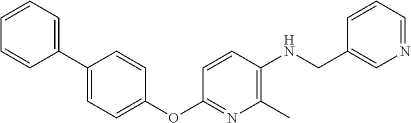 [SMILES: Cc1nc(Oc2ccc(-c3ccccc3)cc2)ccc1NCc1cccnc1]

Following the General procedure H, 6-([1,1′-biphenyl]-4-yloxy)-2-methyl-N-(pyridin-3-ylmethyl)pyridin-3-amine was obtained in 60% yield (0.326 mmol, 120 mg) from 6-([1,1′-biphenyl]-4-yloxy)-2-methylpyridin-3-amine (0.543 mmol, 150 mg).

C₂₄H₂₁N₃O; Mw=367.45 g mol⁻¹; ¹H NMR (400 MHz, CDCl₃) δ 8.67-8.65 (m, 1H), 8.59-8.54 (m, 1H), 7.75-7.71 (m, 1H), 7.58-7.51 (m, 4H), 7.45-7.39 (m, 2H), 7.35-7.29 (m, 2H), 7.12-7.05 (m, 2H), 6.91-6.87 (m, 1H), 6.67-6.63 (m, 1H), 4.40 (s, 2H), 3.83 (s, 1H), 2.40 (s, 3H).

The starting material was prepared as follows:

### Step 1: 6-([1,1′-Biphenyl]-4-yloxy)-2-methyl-3-nitropyridine

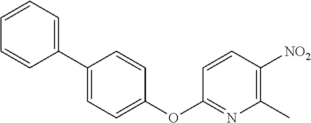 [SMILES: Cc1nc(Oc2ccc(-c3ccccc3)cc2)ccc1[N+](=O)[O-]]

Following the General procedure A, 6-([1,1′-biphenyl]-4-yloxy)-2-methyl-3-nitropyridine was obtained in 89% yield (5.3 mmol, 1.63 g) from [1,1′-biphenyl]-4-ol (5.9 mmol, 1.00 g) and 6-chloro-2-methyl-3-nitropyridine (5.8 mmol, 1.00 g).

C₁₈H₁₄N₂O₃; Mw=306.32 g.mol⁻¹; ¹HNMR (400 MHz, CDCl₃) δ 8.38 (d, J=8.9 Hz, 1H), 7.63 (dd, J=16.5, 7.9 Hz, 4H), 7.46 (t, J=7.6 Hz, 2H), 7.37 (dd, J=8.3, 6.4 Hz, 1H), 7.24 (d, J=8.7 Hz, 2H), 6.84 (d, J=8.9 Hz, 1H), 2.78 (s, 3H).

### Step 2: 6-([1,1′-Biphenyl]-4-yloxy)-2-methylpyridin-3-amine

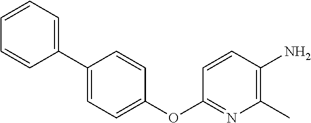 [SMILES: Cc1nc(Oc2ccc(-c3ccccc3)cc2)ccc1N]

Following the General procedure B, 6-([1,1′-biphenyl]-4-yloxy)-2-methylpyridin-3-amine was obtained in 90% yield (4.7 mmol, 1.31 g) from 6-([1,1′-biphenyl]-4-yloxy)-2-methyl-3-nitropyridine (5.3 mmol, 1.61 g).

C₁₈H₁₆N₂O; Mw=276.34 g.mol⁻¹; ¹HNMR (400 MHz, CDCl₃) δ 7.59-7.52 (m, 4H), 7.42 (t, J=7.6 Hz, 2H), 7.32 (ddd, J=7.4, 3.9, 1.2 Hz, 1H), 7.10 (d, J=8.7 Hz, 2H), 7.02 (d, J=8.4 Hz, 1H), 6.63 (d, J=8.4 Hz, 1H), 3.49 (s, 2H), 2.37 (s, 3H).

### Example 24

### 6-((4′-Fluoro-[1,1′-biphenyl]-4-yl)oxy)-N-(pyridin-3-ylmethyl)pyridin-3-amine

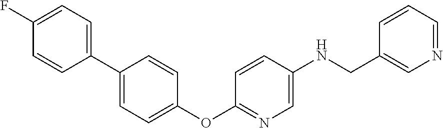 [SMILES: Fc1ccc(-c2ccc(Oc3ccc(NCc4cccnc4)cn3)cc2)cc1]

Following the General procedure H, 6-((4′-fluoro-[1,1′-biphenyl]-4-yl)oxy)-N-(pyridin-3-ylmethyl)pyridin-3-amine was obtained in 51% yield (0.18 mmol, 68 mg) from 6-((4′-fluoro-[1,1′-biphenyl]-4-yl)oxy)pyridin-3-amine (0.35 mmol, 100 mg).

C₂₃H₁₈FN₃O; Mw=371.42 g mol⁻¹; ¹H NMR (400 MHz, CDCl₃) δ 8.66 (s, 1H), 8.56 (d, J=4.9 Hz, 1H), 7.75 (d, J=7.8 Hz, 1H), 7.66 (d, J=2.9 Hz, 1H), 7.59-7.46 (m, 4H), 7.34 (dd, J=7.9, 4.9 Hz, 1H), 7.17-7.00 (m, 5H), 6.84 (dd, J=8.8, 0.6 Hz, 1H), 4.38 (s, 2H), 4.02 (s, 1H).

The starting material was prepared as follows:

### Step 1: 2-(4-Bromophenoxy)-5-nitropyridine

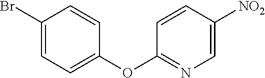 [SMILES: O=[N+]([O-])c1ccc(Oc2ccc(Br)cc2)nc1]

Following the General procedure A, 2-(4-bromophenoxy)-5-nitropyridine was obtained in 89% yield (37.2 mmol, 10.97 g) from 4-bromophenol (42.8 mmol, 7.40 g) and 2-chloro-5-nitropyridine (41.6 mmol, 6.60 g).

C₁₁H₇BrN₂O₃; Mw=295.09 g.mol⁻¹; ¹H NMR (400 MHz, CDCl₃) δ 9.03 (d, J=2.6 Hz, 1H), 8.49 (dd, J=9.0, 2.8 Hz, 1H), 7.57 (d, J=8.8 Hz, 2H), 7.06 (m, 3H).

### Step 2: 24((4′-Fluoro-[1,1′-biphenyl]-4-yl)oxy)-5-nitropyridine

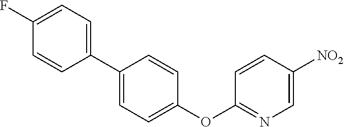 [SMILES: O=[N+]([O-])c1ccc(Oc2ccc(-c3ccc(F)cc3)cc2)nc1]

Following the General procedure E, a mixture of 4-fluorophenylboronic acid (8.9 mmol, 1.25 g), 2-(4-bromophenoxy)-5-nitropyridine (7.8 mmol, 2.30 g), K₂CO₃ (19.5 mmol, 2.70 g) and Pd(PPh₃)₄ (10% mol) in dioxane/H₂O 4:1 (0.05-0.1 M) was converted to 24(4′-fluoro-[1,1′-biphenyl]-4-yl)oxy)-5-nitropyridine in 87% yield (6.8 mmol, 2.10 g).

C₁₇H₁₁FN₂O₃; Mw=310.28 g.mol⁻¹; ¹H NMR (400 MHz, CDCl₃) δ 9.07 (d, J=2.7 Hz, 1H), 8.50 (dd, J=9.0, 2.7 Hz, 1H), 7.62 (d, J=8.5 Hz, 2H), 7.55 (dd, J=8.5, 5.4 Hz, 2H), 7.23 (d, J=8.6 Hz, 2H), 7.14 (t, J=8.6 Hz, 2H), 7.09 (d, J=9.0 Hz, 1H).

### Step 3: 6-((4′-Fluoro-[1,1′-biphenyl]-4-yl)oxy)pyridin-3-amine

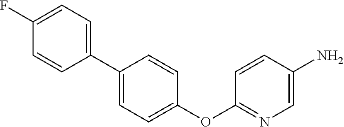 [SMILES: Nc1ccc(Oc2ccc(-c3ccc(F)cc3)cc2)nc1]

Following the General procedure B, 6-((4′-fluoro-[1,1′-biphenyl]-4-yl)oxy)pyridin-3-amine was obtained in 58% yield (3.9 mmol, 1.10 g) from 2-((4′-fluoro-[1,1′-biphenyl]-4-yl)oxy)-5-nitropyridine (6.8 mmol, 2.10 g).

C₁₇H₁₃FN₂O; Mw=280.30 g.mol⁻¹; ¹H NMR (400 MHz, CDCl₃) δ 7.75 (d, J=3.0 Hz, 1H), 7.51 (m, 4H), 7.16-7.07 (m, 5H), 6.82 (d, J=8.6 Hz, 1H).

### Example 25

### 3-Fluoro-4-(4-(pyridin-2-yl)phenoxy)-N-(pyridin-3-ylmethyl)aniline

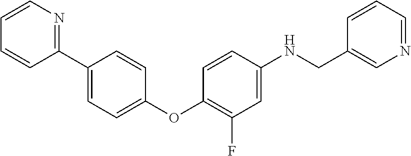 [SMILES: Fc1cc(NCc2cccnc2)ccc1Oc1ccc(-c2ccccn2)cc1]

Following the General procedure H, 3-fluoro-4-(4-(pyridin-2-yl)phenoxy)-N-(pyridin-3-ylmethyl)aniline was obtained in 78% yield (0.27 mmol, 103 mg) from 3-fluoro-4-(4-(pyridin-2-yl)phenoxy)aniline (0.35 mmol, 100 mg).

C₂₃H₁₈FN₃O; Mw=371.42 g mol⁻¹; ¹H NMR (400 MHz, CDCl₃) δ 8.65 (ddd, J=4.8, 1.8, 1.0 Hz, 2H), 8.57 (d, J=4.9 Hz, 1H), 7.99-7.87 (m, 2H), 7.79-7.62 (m, 3H), 7.32 (dd, J=7.9, 4.8 Hz, 1H), 7.19 (ddd, J=7.2, 4.9, 1.4 Hz, 1H), 7.05-6.92 (m, 3H), 6.56-6.35 (m, 2H), 4.37 (s, 2H), 4.16 (s, 1H).

The starting material was prepared as follows:

### Step 1: 2-(4-(2-Fluoro-4-nitrophenoxy)phenyl)pyridine

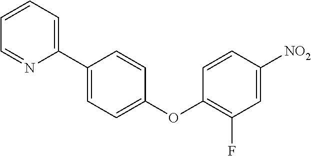 [SMILES: O=[N+]([O-])c1ccc(Oc2ccc(-c3ccccn3)cc2)c(F)c1]

Following the General procedure A, 2-(4-(2-fluoro-4-nitrophenoxy)phenyl)pyridine was obtained in 30% yield (4.38 mmol, 1.36 g) from 4-(pyridin-2-yl)phenol (14.6 mmol, 2.50 g) and 1,2-difluoro-4-nitrobenzene (14.3 mmol, 2.28 g).

C₁₇H₁₁FN₂O₃; Mw=310.28 g.mol⁻¹; ¹H NMR (400 MHz, CDCl₃) δ 8.71 (ddd, J=4.9, 1.8, 0.9 Hz, 1H), 8.12 (dd, J=10.2, 2.6 Hz, 1H), 8.10-8.04 (m, 2H), 8.01 (ddd, J=9.0, 2.6, 1.5 Hz, 1H), 7.84-7.76 (m, 1H), 7.76-7.71 (m, 1H), 7.29 (s, 1H), 7.23-7.16 (m, 2H), 7.06 (dd, J=9.1, 7.9 Hz, 1H).

### Step 2: 3-Fluoro-4-(4-(pyridin-2-yl)phenoxy)aniline

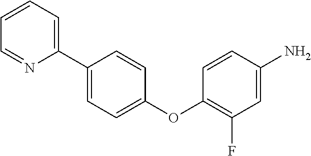 [SMILES: Nc1ccc(Oc2ccc(-c3ccccn3)cc2)c(F)c1]

Following the General procedure B, 3-fluoro-4-(4-(pyridin-2-yl)phenoxy)aniline was obtained in 42% yield (1.8 mmol, 0.51 g) from 2-(4-(2-fluoro-4-nitrophenoxy)phenyl)pyridine (4.38 mmol, 1.36 g).

C₁₇H₁₃FN₂O; Mw=280.30 g.mol⁻¹; ¹H NMR (400 MHz, CDCl₃) δ 8.63 (d, J=5.0 Hz, 1H), 7.90 (d, J=8.5 Hz, 2H), 7.75 (s, 1H), 7.66 (d, J=8.0 Hz, 1H), 7.28 (m, 1H), 7.02-6.94 (m, 2H), 6.90 (t, J=8.8 Hz, 1H), 6.46 (dd, J=12.0, 2.7 Hz, 1H), 6.39 (ddd, J=8.6, 2.7, 1.3 Hz, 1H).

### Example 26

### 3-Fluoro-4-(4-(pyridin-3-yl)phenoxy)-N-(pyridin-3-ylmethyl)aniline

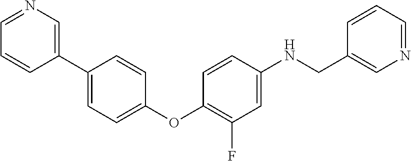 [SMILES: Fc1cc(NCc2cccnc2)ccc1Oc1ccc(-c2cccnc2)cc1]

Following the General procedure H, 3-fluoro-4-(4-(pyridin-3-yl)phenoxy)-N-(pyridin-3-ylmethyl)aniline was obtained in 63% yield (0.22 mmol, 84 mg) from 3-fluoro-4-(4-(pyridin-3-yl)phenoxy)aniline (0.35 mmol, 100 mg).

C₂₃H₁₈FN₃O; Mw=371.42 g mol⁻¹; ¹H NMR (400 MHz, CDCl₃) δ 8.80 (d, J=2.3 Hz, 1H), 8.71-8.51 (m, 3H), 7.83 (ddd, J=7.9, 2.4, 1.6 Hz, 1H), 7.72 (dd, J=7.8, 1.9 Hz, 1H), 7.54-7.43 (m, 2H), 7.33 (ddd, J=12.4, 7.8, 4.8 Hz, 2H), 7.07-6.93 (m, 3H), 6.54-6.34 (m, 2H), 4.37 (s, 2H).

The starting material was prepared as follows:

### Step 1: 3-(4-(2-Fluoro-4-nitrophenoxy)phenyl)pyridine

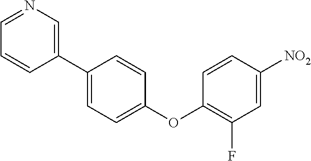 [SMILES: O=[N+]([O-])c1ccc(Oc2ccc(-c3cccnc3)cc2)c(F)c1]

Following the General procedure A, 3-(4-(2-fluoro-4-nitrophenoxy)phenyl)pyridine was obtained in 79% yield (17.7 mmol, 5.48 g) from 4-(pyridin-3-yl)phenol (22.4 mmol, 3.84 g) and 1,2-difluoro-4-nitrobenzene (22.0 mmol, 3.50 g).

C₁₇H₁₁FN₂O₃; Mw=310.28 g.mol⁻¹; ¹H NMR (400 MHz, CDCl₃) δ 8.88 (s, 1H), 8.64 (d, J=5.0 Hz, 1H), 8.13 (dd, J=10.2, 2.7 Hz, 1H), 8.09-7.98 (m, 2H), 7.69-7.61 (m, 2H), 7.55-7.48 (m, 1H), 7.23-7.17 (m, 2H), 7.11 (dd, J=9.0, 7.8 Hz, 1H).

### Step 2: 3-Fluoro-4-(4-(pyridin-3-yl)phenoxy)aniline

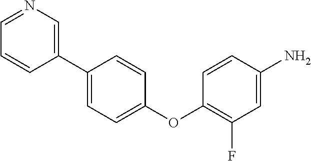 [SMILES: Nc1ccc(Oc2ccc(-c3cccnc3)cc2)c(F)c1]

Following the General procedure B, 3-fluoro-4-(4-(pyridin-3-yl)phenoxy)aniline was obtained in 63% yield (11.1 mmol, 3.1 g) from 3-(4-(2-fluoro-4-nitrophenoxy)phenyl)pyridine (17.7 mmol, 5.48 g).

C₁₇H₁₃FN₂O; Mw=280.30 g.mol⁻¹; ¹H NMR (400 MHz, CDCl₃) δ 8.82 (d, J=2.4 Hz, 1H), 8.57 (dd, J=5.0, 1.5 Hz, 1H), 7.95 (s, 1H), 7.52-7.47 (m, 2H), 7.45 (s, 1H), 7.06-7.00 (m, 2H), 6.97 (t, J=8.7 Hz, 1H), 6.54 (dd, J=12.0, 2.7 Hz, 1H), 6.46 (ddd, J=8.6, 2.7, 1.2 Hz, 1H).

### Example 27

### N-(pyridin-3-ylmethyl)-6-(4-(thiazol-5-yl)phenoxy)pyridin-3-amine

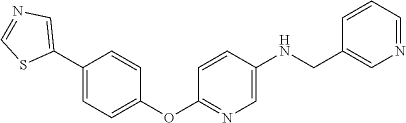 [SMILES: c1cncc(CNc2ccc(Oc3ccc(-c4cncs4)cc3)nc2)c1]

Following the General procedure H, N-(pyridin-3-ylmethyl)-6-(4-(thiazol-5-yl)phenoxy)pyridin-3-amine was obtained in 31% yield (0.11 mmol, 42 mg) from 6-(4-(thiazol-5-yl)phenoxy)pyridin-3-amine (0.37 mmol, 100 mg).

C₂₀H₁₆N₄OS; Mw=360.44 g mol⁻¹; ¹H NMR (400 MHz, CDCl₃) δ 8.72 (s, 1H), 8.68-8.51 (m, 2H), 8.00 (s, 1H), 7.78-7.63 (m, 2H), 7.59-7.50 (m, 2H), 7.30 (d, J=5.8 Hz, 1H), 7.14-7.01 (m, 3H), 6.84 (dd, J=8.7, 0.6 Hz, 1H), 4.36 (s, 2H).

The starting material was prepared as follows:

### Step 1: 5-Nitro-2-(4-(4,4,5,5-tetramethyl-1,3,2-dioxaborolan-2-yl)phenoxy)pyridine

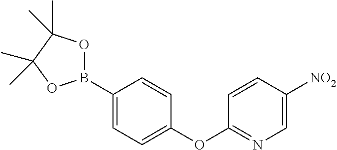 [SMILES: CC1(C)OB(c2ccc(Oc3ccc([N+](=O)[O-])cn3)cc2)OC1(C)C]

To a suspension of 2-(4-bromophenoxy)-5-nitropyridine (20.6 mmol, 6.08 g; expl. 24, Step 1), CH₃COOK (61.1 mmol, 6.00 g) and bis(pinacolato)diboron (30.5 mmol, 7.75 g) in dioxane (0.1 M, 150 mL) under inert atmosphere, Pd(dppf)Cl₂′CH₂Cl₂ (5% mol) was added. The red mixture was stirred 16 h at 105° C. The reaction mixture was filtered through a pad of celite. Water was added to the filtrate and the mixture was extracted with EtOAc (2x). The organic layer was washed with sat. aq. NaHCO₃, brine, dried over Na₂SO₄, filtered-off and concentrated. The crude product was purified by combi flash column chromatography using EtOAc/cyclohexane (1% to 20%) as the eluent, to afford 5-nitro-2-(4-(4,4,5,5-tetramethyl-1,3,2-dioxaborolan-2-yl)phenoxy)pyridine (15.6 mmol, 5.93 g, 76% yield).

C₁₇H₁₉BN₂O₅; Mw=342.16 g.mol⁻¹; ¹H NMR (400 MHz, CDCl₃) δ 9.04 (d, J=2.5 Hz, 1H), 8.47 (dd, J=9.1, 2.8 Hz, 1H), 7.94-7.88 (m, 2H), 7.20-7.13 (m, 2H), 7.06-7.01 (m, 1H), 1.35 (s, 12H).

### Step 2: 5-(4-((5-Nitropyridin-2-yl)oxy)phenyl)thiazole

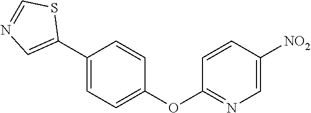 [SMILES: O=[N+]([O-])c1ccc(Oc2ccc(-c3cncs3)cc2)nc1]

Following the General procedure D, a mixture of 5-nitro-2-(4-(4,4,5,5-tetramethyl-1,3,2-dioxaborolan-2-yl)phenoxy)pyridine (9.2 mmol, 3.50 g), 5-bromothiazole (7.4 mmol, 1.25 g), Cs₂CO₃ (15.0 mmol, 4.90 g) and PdCl₂(PPh₃)₂ (10% mol) in dioxane/H₂O 4:1 (0.05-0.1 M) was converted to 5-(4-((5-nitropyridin-2-yl)oxy)phenyl)thiazole in 52% yield (3.8 mmol, 1.15 g).

C₁₄H₉N₃O₃S; Mw=299.30 g.mol⁻¹; ¹H NMR (400 MHz, CDCl₃) δ 9.05 (d, J=2.6 Hz, 1H), 8.78 (s, 1H), 8.51 (dd, J=9.1, 2.8 Hz, 1H), 8.08 (s, 1H), 7.67 (d, J=8.2 Hz, 2H), 7.23 (d, J=8.4 Hz, 2H), 7.10 (d, J=9.0 Hz, 1H).

### Step 3: 6-(4-(Thiazol-5-yl)phenoxy)pyridin-3-amine

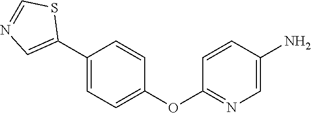 [SMILES: Nc1ccc(Oc2ccc(-c3cncs3)cc2)nc1]

Following the General procedure B, 6-(4-(thiazol-5-yl)phenoxy)pyridin-3-amine was obtained in 46% yield (1.8 mmol, 0.48 g) from 5-(4-((5-nitropyridin-2-yl)oxy)phenyl)thiazole (3.8 mmol, 1.15 g).

C₁₄H₁₁N₃OS; Mw=269.32 g.mol⁻¹; ¹H NMR (400 MHz, CDCl₃) δ 8.67 (s, 1H), 7.95 (s, 1H), 7.67 (d, J=3.0 Hz, 1H), 7.49 (d, J=8.7 Hz, 2H), 7.05 (m, 3H), 6.76 (d, J=8.6 Hz, 1H).

### Example 28

### 4-((2,2′-Dimethyl-[1,1′-biphenyl]-4-yl)oxy)-N-(pyridin-3-ylmethy)aniline

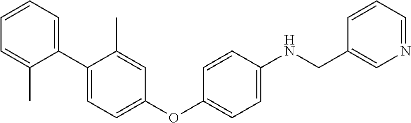 [SMILES: Cc1ccccc1-c1ccc(Oc2ccc(NCc3cccnc3)cc2)cc1C]

Following the General procedure H, 4-((2,2′-dimethyl-[1,1′-biphenyl]-4-yl)oxy)-N-(pyridin-3-ylmethyl)aniline was obtained in 51% yield (0.15 mmol, 68 mg) from 44((2,2′-dimethyl-[1,1′-biphenyl]-4-yl)oxy)aniline (0.34 mmol, 100 mg).

C₂₆H₂₄N₂O; Mw=380.49 g mol⁻¹; ¹H NMR (400 MHz, CDCl₃) δ 8.67 (dd, J=2.3, 0.8 Hz, 1H), 8.56 (dd, J=4.9, 1.7 Hz, 1H), 7.83-7.75 (m, 1H), 7.34 (ddd, J=7.9, 4.9, 0.8 Hz, 1H), 7.26-7.23 (m, 2H), 7.23-7.18 (m, 1H), 7.12-7.06 (m, 1H), 7.03-6.92 (m, 3H), 6.87-6.83 (m, 1H), 6.76 (ddd, J=8.3, 2.6, 0.6 Hz, 1H), 6.68-6.61 (m, 2H), 4.39 (s, 2H), 2.06 (s, 3H), 2.00 (s, 3H).

The starting material was prepared as follows:

### Step 1: 1-Bromo-2-methyl-4-(4-nitrophenoxy)benzene

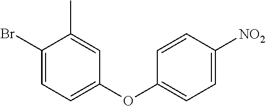 [SMILES: Cc1cc(Oc2ccc([N+](=O)[O-])cc2)ccc1Br]

Following the General procedure A, 1-bromo-2-methyl-4-(4-nitrophenoxy)benzene was obtained in 91% yield (27.1 mmol, 8.34 g) from 4-bromo-3-methyl-phenol (31.5 mmol, 6.01 g) and 4-fluoro-nitrobenzene (29.9 mmol, 4.22 g).

C₁₃H₁₀BrNO₃; Mw=308.13 g.mol⁻¹; ¹H NMR (400 MHz, CDCl₃) δ 8.24-8.18 (m, 2H), 7.57 (d, J=8.6 Hz, 1H), 7.05-6.96 (m, 3H), 6.80 (dd, J=8.6, 2.9 Hz, 1H), 2.41 (s, 3H).

### Step 2: 2,2′-Dimethyl-4-(4-nitrophenoxy)-1,1′-biphenyl

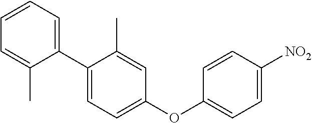 [SMILES: Cc1ccccc1-c1ccc(Oc2ccc([N+](=O)[O-])cc2)cc1C]

Following the General procedure E, a mixture of o-tolylboronic acid (30.9 mmol, 4.20 g), 1-bromo-2-methyl-4-(4-nitrophenoxy)benzene (27.1 mmol, 8.34 g), K₂CO₃ (55.0 mmol, 7.60 g) and Pd(PPh₃)₄ (10% mol) in dioxane/H₂O 4:1 (0.05-0.1 M) was converted to 2,2′-dimethyl-4-(4-nitrophenoxy)-1,1′-biphenyl in 92% yield (24.9 mmol, 7.96 g). C₂₀H₁₇NO₃; Mw=319.36 g.mol⁻¹; ¹H NMR (400 MHz, CDCl₃) δ 8.26-8.21 (m, 2H), 7.31-7.27 (m, 2H), 7.24 (d, J=4.8 Hz, 1H), 7.14 (dd, J=14.2, 7.6 Hz, 2H), 7.11-7.04 (m, 2H), 7.00 (d, J=2.5 Hz, 1H), 6.95 (dd, J=8.2, 2.2 Hz, 1H), 2.09 (s, 3H), 2.07 (s, 3H).

### Step 3: 4-((2,2′-Dimethyl-[1,1′-biphenyl]-4-yl)oxy)aniline

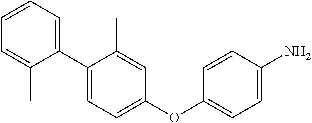 [SMILES: Cc1ccccc1-c1ccc(Oc2ccc(N)cc2)cc1C]

Following the General procedure B, 4-((2,2′-dimethyl-[1,1′-biphenyl]-4-yl)oxy)aniline was obtained in 88% yield (21.9 mmol, 6.33 g) from 2,2′-dimethyl-4-(4-nitrophenoxy)-1,1′-biphenyl (24.9 mmol, 7.96 g).

C₂₀H₁₉NO; Mw=289.38 g.mol⁻¹; ¹H NMR (400 MHz, CDCl₃) δ 7.26-7.24 (m, 2H), 7.23-7.18 (m, 1H), 7.10 (d, J=6.8 Hz, 1H), 7.00 (d, J=8.3 Hz, 1H), 6.96-6.91 (m, 2H), 6.84 (d, J=2.5 Hz, 1H), 6.77 (dd, J=8.4, 2.6 Hz, 1H), 6.75-6.71 (m, 2H), 2.07 (s, 3H), 2.00 (s, 3H).

### Example 29

### 6-((2,2′-dimethyl-[1,1′-biphenyl]-4-yl)oxy)-N-(pyridin-3-ylmethyl)pyridin-3-amine

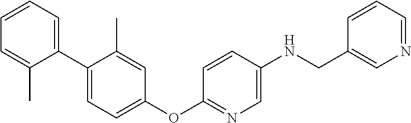 [SMILES: Cc1ccccc1-c1ccc(Oc2ccc(NCc3cccnc3)cn2)cc1C]

Following the General procedure H, 6-((2,2′-dimethyl-[1,1′-biphenyl]-4-yl)oxy)-N-(pyridin-3-ylmethyl)pyridin-3-amine was obtained in 49% yield (0.17 mmol, 65 mg) from 6-((2,2′-dimethyl-[1,1′-biphenyl]-4-yl)oxy)pyridin-3-amine (0.345 mmol, 100 mg).

C₂₅H₂₃N₃O; Mw=381.48 g mol⁻¹; ¹H NMR (400 MHz, CDCl₃) δ 8.67 (s, 1H), 8.60-8.56 (m 1H), 7.76-7.72 (m, 2H), 7.36-7.31 (m, 1H), 7.30-7.26 (m, 2H), 7.24-7.20 (m, 1H), 7.15-7.11 (m, 1H), 7.10-7.04 (m, 2H), 6.99-6.96 (m, 1H), 6.94-6.89 (m, 1H), 6.87-6.84 (m, 1H), 4.40 (s, 2H), 4.02 (s, 1H), 2.10 (s, 3H), 2.04 (s, 3H).

The starting material was prepared as follows:

### Step 1: 2-(4-Bromo-3-methylphenoxy)-5-nitropyridine

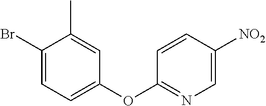 [SMILES: Cc1cc(Oc2ccc([N+](=O)[O-])cn2)ccc1Br]

Following the General procedure A, 2-(4-bromo-3-methylphenoxy)-5-nitropyridine was obtained in 98% yield (6.2 mmol, 1.93 g) from 4-bromo-3-methyl-phenol (6.8 mmol, 1.28 g) and 2-chloro-5-nitropyridine (6.4 mmol, 1.01 g).

C₁₂H₉BrN₂O₃; Mw=309.12 g.mol⁻¹; ¹H NMR (400 MHz, CDCl₃) δ 9.04 (d, J=2.7 Hz, 1H), 8.49 (dd, J=9.0, 2.9 Hz, 1H), 7.59 (d, J=8.6 Hz, 1H), 7.10-7.01 (m, 2H), 6.88 (dd, J=8.6, 2.8 Hz, 1H), 2.42 (s, 3H).

### Step 2: 24((2,2′-Dimethyl-[1,1′-biphenyl]-4-yl)oxy)-5-nitropyridine

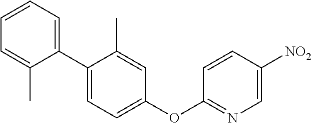 [SMILES: Cc1ccccc1-c1ccc(Oc2ccc([N+](=O)[O-])cn2)cc1C]

Following the General procedure E, a mixture of o-tolylboronic acid (1.9 mmol, 264 mg), 2-(4-bromo-3-methylphenoxy)-5-nitropyridine (1.3 mmol, 400 mg), K₂CO₃ (2.6 mmol, 358 mg) and Pd(PPh₃)₄ (10% mol) in dioxane/H₂O 4:1 (0.05-0.1 M) was converted to 2-((2,2′-dimethyl-[1,1′-biphenyl]-4-yl)oxy)-5-nitropyridine in 95% yield (1.2 mmol, 392 mg).

C₁₉H₁₆N₂O₃; Mw=320.35 g.mol⁻¹; ¹H NMR (400 MHz, CDCl₃) δ 9.11 (d, J=2.8 Hz, 1H), 8.49 (dd, J=9.1, 2.8 Hz, 1H), 7.35-7.31 (m, 1H), 7.29 (m, 1H), 7.25-7.22 (m, 1H), 7.18 (d, J=8.2 Hz, 1H), 7.15 (dt, J=7.0, 1.2 Hz, 1H), 7.09-7.00 (m, 3H), 2.10 (s, 3H), 2.09 (s, 3H).

### Step 3: 6-((2,2′-Dimethyl-[1,1′-biphenyl]-4-yl)oxy)pyridin-3-amine

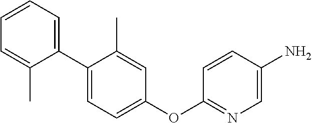 [SMILES: Cc1ccccc1-c1ccc(Oc2ccc(N)cn2)cc1C]

Following the General procedure B, 6-((2,2′-dimethyl-[1,1′-biphenyl]-4-yl)oxy)pyridin-3-amine was obtained in 37% yield (0.5 mmol, 132 mg) from 2-((2,2′-dimethyl-[1,1′-biphenyl]-4-yl)oxy)-5-nitropyridine (1.2 mmol, 392 mg).

C₁₉H₁₈N₂O; Mw=290.37 g.mol⁻¹; ¹H NMR (400 MHz, CDCl₃) δ 7.79 (d, J=2.8 Hz, 1H), 7.25 (s, 2H), 7.23-7.18 (m, 1H), 7.12 (t, J=6.2 Hz, 2H), 7.06 (d, J=8.2 Hz, 1H), 6.96 (d, J=1.9 Hz, 1H), 6.90 (dd, J=8.3, 2.3 Hz, 1H), 6.82 (d, J=8.6 Hz, 1H), 2.85 (s, 2H), 2.08 (s, 3H), 2.02 (s, 3H).

### Example 30

### (4′-Fluoro-[1,1′-biphenyl]-4-yl)(4-((pyridin-3-ylmethyl)amino)phenyl)methanol

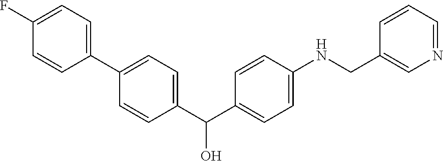 [SMILES: OC(c1ccc(NCc2cccnc2)cc1)c1ccc(-c2ccc(F)cc2)cc1]

Following the General procedure H, (4′-fluoro-[1,1′-biphenyl]-4-yl)(4-((pyridin-3-ylmethyl)-amino)phenyl)methanol was obtained in 78% yield with purification (0.20 mmol, 0.078 g) from (4-aminophenyl)(4′-fluoro-[1,1′-biphenyl]-4-yl)methanol (0.51 mmol, 0.150 g).

C₂₅H₂₁FN₂O; Mw=384.45 g.mol⁻¹; ¹H NMR (400 MHz, CDCl₃) δ 8.65-8.58 (m, 1H), 8.52 (dd, J=4.8, 1.6 Hz, 1H), 7.69 (dt, J=7.9, 1.9 Hz, 1H), 7.55-7.45(m, 4H), 7.47-7.41 (m, 2H), 7.30-7.26 (m, 1H), 7.23-7.17 (m, 2H), 7.16-7.07 (m, 2H), 6.66-6.57 (m, 2H), 5.80 (s, 1H), 4.36 (s, 2H).

The starting material was prepared as follows:

### Step 1: (4′-Fluoro-[1,1′-biphenyl]-4-yl)boronic acid

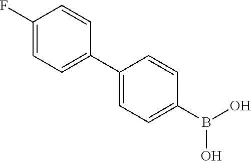 [SMILES: OB(O)c1ccc(-c2ccc(F)cc2)cc1]

To a solution of 4-bromo-4′-fluoro-1,1′-biphenyl (1.99 mmol, 0.500 g), in dry THF (19.9 ml), at −78° C., under inert atmosphere, tert-butyllithium (2.390 mmol, 1.4 ml) was added dropwise. The reaction mixture was stirred at −78° C. for 20 min before trimethyl borate (1.99 mmol, 0.222 ml) was added dropwise. After stirring for 1 h the reaction mixture was brought to RT and quenched with 1N HCl and stirred for 30 min. The reaction mixture was concentrated under vacuo and the observed precipitate was filtered off and air dried to get (4′-fluoro-[1,1′-biphenyl]-4-yl)boronic acid (1.852 mmol, 0.400 g) in 93% yield as a white powder. The NMR spectra was identical to the previously reported one (Neya, et al., WO 2003 022842).

### Step 2: (4′-Fluoro-[1,1′-biphenyl]-4-yl)(4-nitrophenyl)methanol

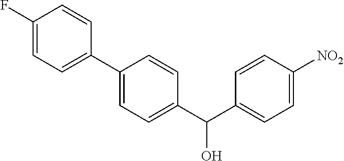 [SMILES: O=[N+]([O-])c1ccc(C(O)c2ccc(-c3ccc(F)cc3)cc2)cc1]

To a solution of chloro(1,5-cyclooctadiene)rhodium(I) dimer (0.093 mmol, 0.046 g) in dry dioxane (12.3 ml), at RT, under inert atmosphere, was added potassium hydroxide (1.852 mmol, 1.234 ml) and the mixture stirred for 3 min. To this solution (4′-fluoro-[1,1′-biphenyl]-4-yl)boronic acid (1.852 mmol, 0.400 g) was added followed by 4-nitrobenzaldehyde (3.760 mmol, 0.560 g). The mixture was stirred for 14 h at RT and then quenched by addition of brine. The mixture was extracted with EtOAc (3×). The combined organic layers were dried over Na₂SO₄, filtered and concentrated under reduced pressure. The residue was purified by flash chromatography (Biotage KP-Sil 50 g, hexane/EtOAc, 0-20%) to afford (4′-fluoro-[1,1′-biphenyl]-4-yl)(4-nitrophenyl)methanol (1.46 mmol, 0.473 g) in 79% yield as a white solid. C₁₉H₁₄FNO₃; Mw=323.32 g.mol⁻¹; ¹H NMR (400 MHz, CDCl₃) δ 8.25-8.16 (m, 2H), 7.65-7.59 (m, 2H), 7.56-7.48 (m, 4H), 7.44-7.39 (m, 2H), 7.17-7.06 (m, 2H), 5.97 (s, 1H), 2.41 (s, 1H). ¹⁹F NMR (377 MHz, CDCl₃) δ-115.19.

### Step 3: (4-Aminophenyl)(4′-fluoro-[1,1′-biphenyl]-4-yl)methanol

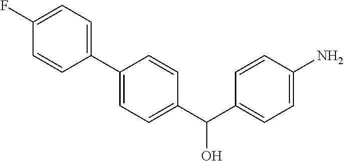 [SMILES: Nc1ccc(C(O)c2ccc(-c3ccc(F)cc3)cc2)cc1]

Following the General procedure G, (4-aminophenyl)(4′-fluoro-[1,1′-biphenyl]-4-yl)methanol was obtained in 98% yield with purification (1.55 mmol, 0.445 g) from (4′-fluoro-[1,1′-biphenyl]-4-yl)(4-nitrophenyl)methanol (1.52 mmol, 0.500 g).

C₁₉H₁₆FNO; Mw=293.33 g.mol⁻¹; ¹H NMR (400 MHz, CDCl₃) δ 7.56-7.48 (m, 4H), 7.47-7.41 (m, 2H), 7.22-7.15 (m, 2H), 7.13-7.07 (m, 2H), 6.71-6.60 (m, 2H), 5.80 (s, 1H).

### Example 31

### 4-((4′-Fluoro-[1,1′-biphenyl]-4-yl)(methoxy)methyl)-N-(pyridin-3-ylmethyl)aniline

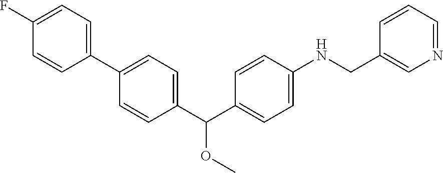 [SMILES: COC(c1ccc(NCc2cccnc2)cc1)c1ccc(-c2ccc(F)cc2)cc1]

Following the General procedure H, 4-((4′-fluoro-[1,1′-biphenyl]-4-yl)(methoxy)methyl)-N-(pyridin-3-ylmethyl)aniline was obtained in 52% yield with purification (0.25 mmol, 0.102 g) from 4-((4′-fluoro-[1,1′-biphenyl]-4-yl)(methoxy)methyl)aniline (0.51 mmol, 0.150 g).

C₂₆H₂₃FN₂O; Mw=398.47 g.mol⁻¹; ¹H NMR (400 MHz, CDCl₃) δ 8.62 (d, J=2.2 Hz, 1H), 8.52 (dd, J=4.9, 1.6 Hz, 1H), 7.69 (dtd, J=7.8, 1.7, 1.0 Hz, 1H), 7.55-7.46 (m, 4H), 7.43-7.36 (m, 2H), 7.26 (s, 1H), 7.19-7.14 (m, 2H), 7.13-7.07 (m, 2H), 6.62-6.57 (m, 2H), 5.19 (s, 1H), 4.35 (s, 2H), 3.38 (s, 3H).

The starting material was prepared as follows:

### Step 1: 4-Fluoro-4′-(methoxy(4-nitrophenyl)methyl)-1,1′-biphenyl

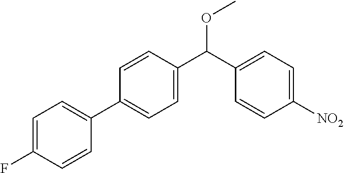 [SMILES: COC(c1ccc(-c2ccc(F)cc2)cc1)c1ccc([N+](=O)[O-])cc1]

To a solution of (4′-fluoro-[1,1′-biphenyl]-4-yl)(4-nitrophenyl)methanol (0.464 mmol, 0.15 g; expl. 30, Step 2), in acetone (4.64 ml), was added Cs₂CO₃ (1.392 mmol, 0.453 g) followed by iodomethane (0.696 mmol, 0.044 ml). The reaction mixture was refluxed for 4 h in a sealed tube at 60° C. The cooled reaction mixture was directly loaded on silica and the residue purified by flash chromatograpy (Biotage KP-Sil 25 g, hexane/EtOAc, 0-20%) to afford 4-fluoro-4′-(methoxy(4-nitrophenyl)methyl)-1,1′-biphenyl (0.406 mmol, 0.137 g) in 88% yield.

C₂₀H₁₆FNO₃; Mw=337.34 g.mol⁻¹; ¹H NMR (400 MHz, CDCl₃) δ 8.22-8.17 (m, 2H), 7.61-7.48 (m, 6H), 7.41-7.36 (m, 2H), 7.15-7.07 (m, 2H), 5.36 (s, 1H), 3.43 (s, 3H).

### Step 2: 4-((4′-Fluoro-[1,1′-biphenyl]-4-yl)(methoxy)methyl)aniline

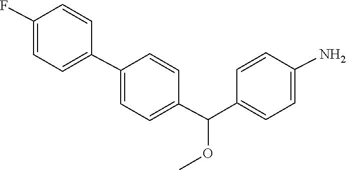 [SMILES: COC(c1ccc(N)cc1)c1ccc(-c2ccc(F)cc2)cc1]

Following the General procedure G, 4-((4′-fluoro-[1,1′-biphenyl]-4-yl)(methoxy)methyl)-aniline was obtained in 78% yield with purification (1.30 mmol, 0.400 g) from 4-fluoro-4′-(methoxy(4-nitrophenyl)methyl)-1,1′-biphenyl (1.78 mmol, 0.600 g).

C₂₀H₁₈FNO; Mw=307.37 g.mol⁻¹; ¹H NMR (300 MHz, Chloroform-d) δ 7.59-7.44 (m, 4H), 7.45-7.33 (m, 2H), 7.22-7.00 (m, 4H), 6.76-6.55 (m, 2H), 5.19 (s, 1H), 3.38 (s, 3H).

### Example 32

### 4-((4′-Fluoro-[1,1′-biphenyl]-4-yl)methyl)-N-(pyridin-3-ylmethyl)aniline

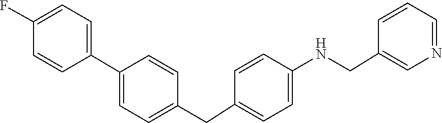 [SMILES: Fc1ccc(-c2ccc(Cc3ccc(NCc4cccnc4)cc3)cc2)cc1]

Following the General procedure H, 4-((4′-fluoro-[1,1′-biphenyl]-4-yl)methyl)-N-(pyridin-3-ylmethyl)aniline was obtained in 87% yield (0.27 mmol, 115 mg) from 4-((4′-fluoro-[1,1′-biphenyl]-4-yl)methyl)aniline (0.36 mmol, 100 mg).

C₂₅H₂₁FN₂; Mw=368.46 g mol⁻¹; ¹H NMR (400 MHz, CDCl₃) δ 8.64 (s, 1H), 8.54 (d, J=4.9 Hz, 1H), 7.76 (d, J=8.0 Hz, 1H), 7.55-7.47 (m, 2H), 7.48-7.41 (m, 2H), 7.31 (t, J=6.3 Hz, 1H), 7.26-7.21 (m, 3H), 7.15-7.07 (m, 2H), 7.06-7.00 (m, 2H), 6.62-6.54 (m, 2H), 4.37 (s, 2H), 3.91 (s, 2H).

The starting material was prepared as follows:

### Step 1: 4-Fluoro-4′-(fluoro(4-nitrophenyl)methyl)-1,1′-biphenyl

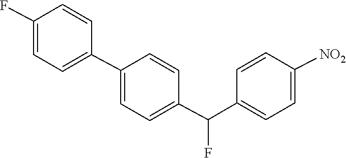 [SMILES: O=[N+]([O-])c1ccc(C(F)c2ccc(-c3ccc(F)cc3)cc2)cc1]

To a solution of (4′-fluoro-[1,1′-biphenyl]-4-yl)(4-nitrophenyl)methanol (1.54 mmol, 0.500 g; expl. 30, Step 2), in dry DCM (7.73 ml), under inert atmosphere, at −78° C., was added dropwise diethylamino-sulfur-trifluoride (1.85 mmol, 0.245 ml). The reaction mixture was stirred at the same temperature for 2 h then brought to RT. The reaction was then quenched using saturated NaHCO₃ solution. The two layers were separated, and the aqueous layer was extracted with DCM (3×). The combined organic layers were dried over Na₂SO₄, filtered and concentrated under reduced pressure to afford 4-fluoro-4′-(fluoro(4-nitrophenyl)methyl)-1,1′-biphenyl (1.38 mmol, 0.450 g) in 89% yield as yellow solid.

C₁₉H₁₃F₂NO₂; Mw=325.31 g.mol⁻¹; ¹H NMR (400 MHz, CDCl₃) δ 8.29-8.22 (m, 2H), 7.59-7.49 (m, 6H), 7.42-7.36 (m, 2H), 7.17-7.09 (m, 2H), 6.58 (d, J=47.0 Hz, 1H).

### Step 2: 4-((4′-Fluoro-[1,1′-biphenyl]-4-yl)methyl)aniline

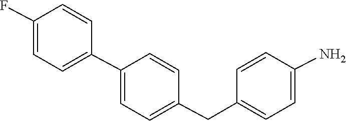 [SMILES: Nc1ccc(Cc2ccc(-c3ccc(F)cc3)cc2)cc1]

Following the General procedure G, 4((4′-fluoro-[1,1′-biphenyl]-4-yl)methyl)aniline was obtained in 60% yield with purification (1.30 mmol, 0.154 g) from 4-fluoro-4′-(fluoro(4-nitrophenyl)methyl)-1,1′-biphenyl (0.92 mmol, 0.300 g).

C₁₉H₁₆FN; Mw=277.34 g.mol⁻¹; ¹H NMR (400 MHz, CDCl₃) δ 7.56-7.50 (m, 2H), 7.49-7.44 (m, 2H), 7.27-7.24 (m, 2H), 7.16-7.09 (m, 2H), 7.06-7.01 (m, 2H), 6.71-6.66 (m, 2H), 3.94 (s, 2H).

### Example 33

### 6-((6-(4-Fluorophenyl)pyridin-3-yl)oxy)-2-methyl-N-(pyridin-3-ylmethyl)pyridin-3-amine

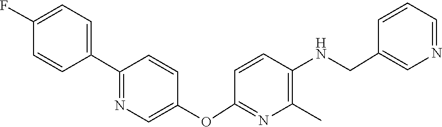 [SMILES: Cc1nc(Oc2ccc(-c3ccc(F)cc3)nc2)ccc1NCc1cccnc1]

Following the General procedure H, 6-((6-(4-fluorophenyl)pyridin-3-yl)oxy)-2-methyl-N-(pyridin-3-ylmethyl)pyridin-3-amine was obtained in 20% yield with purification (0.22 mmol, 86 mg) from 6-((6-(4-fluorophenyl)pyridin-3-yl)oxy)-2-methylpyridin-3-amine (1.155 mmol, 0.341 g) and nicotinaldehyde (1.60 mmol, 1.40 eq.).

C₂₃H₁₉FN₄O; MW=386.43 g mol-1; ¹H NMR (400 MHz, DMSO-d₆) δ 8.60 (s, 1H), 8.44 (d, J=4.7 Hz, 1H), 8.39-8.34 (m, 1H), 8.10-8.05 (m, 2H), 7.92 (d, J=8.7 Hz, 1H), 7.77 (d, J=7.8 Hz, 1H), 7.47 (dd, J=8.7, 2.8 Hz, 1H), 7.36-7.26 (m, 3H), 6.98-6.90 (m, 1H), 6.76 (d, J=8.5 Hz, 1H), 5.88 (t, J=6.1 Hz, 1H), 4.40 (d, J=6.0 Hz, 2H), 2.29 (s, 3H).

The starting material was prepared as follows:

### Step 1: 2-Bromo-5-(methoxymethoxy)pyridine

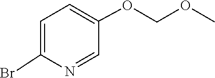

To a solution of 6-bromopyridin-3-ol (29 mmol, 5.0 g), in dry DMF (29 mL), under N₂, at 0° C., was added portionwise NaH (29 mmol, 1.1 g, 60% wt). The mixture was stirred at 0° C. for 1 h. Methyl chloromethyl ether (29 mmol, 2.3 g, 2.2 mL,) was then slowly added. The mixture was stirred at 0° C. for 1 h and then allowed to warm to RT over the weekend. The reaction mixture was cooled to 0° C. and saturated NaHCO₃ solution was added. The mixture was warmed to RT and diluted with H₂O. The mixture was extracted with AcOEt (3×). The combined organic layers were washed with H₂O (3×) and brine. The mixture was dried over MgSO₄, filtered and concentrated under reduced pressure. The product, 2-bromo-5-(methoxymethoxy)pyridine (29 mmol, 6.3 g), was isolated as a colorless oil in quantitative yield.

C₇H₈BrNO₂; Mw=218.05 g mol⁻¹; ¹H NMR (400 MHz, CDCl₃) δ 8.15 (dd, J=3.1, 0.6 Hz, 1H), 7.35 (dd, J=8.7, 0.6 Hz, 1H), 7.27-7.20 (m, 1H), 5.15 (s, 2H), 3.46 (s, 3H).

### Step 2: 2-(4-Fluorophenyl)-5-(methoxymethoxy)pyridine

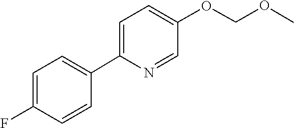 [SMILES: COCOc1ccc(-c2ccc(F)cc2)nc1]

Following the General procedure D, a mixture of (4-fluorophenyl)boronic acid (32.0 mmol, 4.4 g), 2-bromo-5-(methoxymethoxy)pyridine (29.0 mmol, 6.3 g), K₂CO₃ (58.0 mmol, 8.0 g), and Pd(PPh₃)₂Cl₂ (2.9 mmol, 2.0 g) in 4:1 mixture of 2-propanol/H₂O (0.1 M) was converted to 2-(4-fluorophenyl)-5-(methoxymethoxy)pyridine in 56% yield (16.20 mmol, 3.79 g).

C₁₃H₁₂FNO₂; Mw=233.24 g mol⁻¹; ¹H NMR (400 MHz, CDCl₃) δ 8.46 (dd, J=2.9, 0.7 Hz, 1H), 7.97-7.83 (m, 2H), 7.60 (dd, J=8.7, 0.7 Hz, 1H), 7.43 (dd, J=8.7, 2.9 Hz, 1H), 7.13 (dd, J=8.9, 8.5 Hz, 2H), 5.23 (s, 2H), 3.51 (s, 3H).

### Step 3: 6-(4-Fluorophenyl)pyridin-3-ol

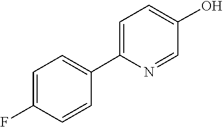 [SMILES: Oc1ccc(-c2ccc(F)cc2)nc1]

To a solution of 2-(4-fluorophenyl)-5-(methoxymethoxy)pyridine (16.20 mmol, 3.79 g), in dioxane (20 mL), at RT was added a 4M HCl (146 mmol, 36.6 mL) dioxane solution. The mixture was heated at 80° C. overnight. The reaction mixture was cooled to RT and concentrated under reduced pressure. The residue was dissolved in H₂O and extracted with DCM (3×). The aqueous layer was neutralized to pH=6-7 with solid Na₂CO₃. The precipitated white solid was filtered off, washed with hexane and dried under vacuo for 2 h, to afford 6-(4-fluorophenyl)pyridin-3-ol (15.0 mmol, 2.83 g) in 92% yield.

C₁₁H₈FNO; Mw=189.19 g mol⁻¹; ¹H NMR (400 MHz, DMSO-d₆) δ 10.04 (s, 1H), 8.20 (dd, J=2.9, 0.7 Hz, 1H), 8.09-7.93 (m, 2H), 7.78 (dd, J=8.7, 0.7 Hz, 1H), 7.33-7.15 (m, 3H).

### Step 4: 6-((6-(4-Fluorophenyl)pyridin-3-yl)oxy)-2-methyl-3-nitropyridine

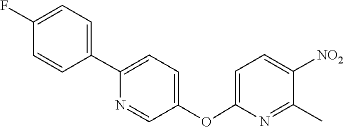 [SMILES: Cc1nc(Oc2ccc(-c3ccc(F)cc3)nc2)ccc1[N+](=O)[O-]]

Following the General procedure A, 6-((6-(4-fluorophenyl)pyridin-3-yl)oxy)-2-methyl-3-nitropyridine was obtained in 100% yield (3.00 mmol, 900 mg) from 6-(4-fluorophenyl)pyridin-3-ol (3.00 mmol, 600 mg) and 6-chloro-2-methyl-3-nitropyridine (3.00 mmol, 500 mg).

C₁₇H₁₂FN₃O₃; Mw=325.29 g mol⁻¹; ¹H NMR (400 MHz, CDCl₃) δ 8.58 (dd, J=2.7, 0.7 Hz, 1H), 8.42 (d, J=8.9 Hz, 1H), 8.03-7.96 (m, 2H), 7.76 (dd, J=8.7, 0.7 Hz, 1H), 7.60 (dd, J=8.7, 2.7 Hz, 1H), 7.20-7.14 (m, 2H), 6.96 (dd, J=8.9, 0.7 Hz, 1H), 2.72 (s, 3H).

### Step 5: 6-((6-(4-Fluorophenyl)pyridin-3-yl)oxy)-2-methylpyridin-3-amine

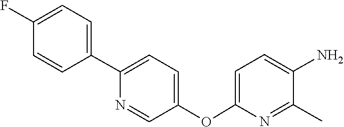 [SMILES: Cc1nc(Oc2ccc(-c3ccc(F)cc3)nc2)ccc1N]

Following the General procedure B, 6-((6-(4-fluorophenyl)pyridin-3-yl)oxy)-2-methylpyridin-3-amine was obtained in 89% yield (2.80 mmol, 732 mg) from 6-((6-(4-fluorophenyl)pyridin-3-yl)oxy)-2-methyl-3-nitropyridine (2.80 mmol, 910 mg).

C₁₇H₁₄FN₃O; Mw=295.31 g mol⁻¹; ¹H NMR (400 MHz, CDCl₃) δ 8.49 (dd, J=2.8, 0.7 Hz, 1H), 7.98-7.88 (m, 2H), 7.64 (dd, J=8.4, 0.7 Hz, 1H), 7.45 (dd, J=8.7, 2.8 Hz, 1H), 7.19-7.08 (m, 2H), 7.04 (d, J=8.4 Hz, 1H), 6.68 (dd, J=8.4, 0.7 Hz, 1H), 3.50 (s, 2H), 2.33 (s, 3H).

### Example 34

### 6-((6-(4-Fluorophenyl)pyridin-3-yl)oxy)-4-methyl-N-(pyridin-3-ylmethyl)pyridin-3-amine

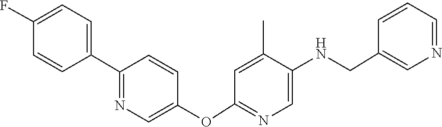 [SMILES: Cc1cc(Oc2ccc(-c3ccc(F)cc3)nc2)ncc1NCc1cccnc1]

Following the General procedure H, 6-((6-(4-fluorophenyl)pyridin-3-yl)oxy)-4-methyl-N-(pyridin-3-ylmethyl)pyridin-3-amine was obtained in 84% yield after purification (0.964 mmol, 0.373 g) from 6-((6-(4-fluorophenyl)pyridin-3-yl)oxy)-4-methylpyridin-3-amine (1.14 mmol, 0.337 g) and nicotinaldehyde (1.617 mmol, 1.40 eq.).

C₂₃H₁₉FN₄O; MW=386.43 g mol-1; ¹H NMR (400 MHz, DMSO-d₆) δ 8.60 (d, J=1.8 Hz, 1H), 8.44 (dd, J=4.8, 1.5 Hz, 1H), 8.37 (d, J=2.7 Hz, 1H), 8.11-8.02 (m, 2H), 7.91 (d, J=8.7 Hz, 1H), 7.77 (d, J=7.8 Hz, 1H), 7.50 (dd, J=8.7, 2.8 Hz, 1H), 7.37-7.24 (m, 4H), 6.89 (s, 1H), 5.80 (t, J=6.1 Hz, 1H), 4.39 (d, J=6.1 Hz, 2H), 2.24 (s, 3H).

The starting material was prepared as follows:

### Step 1: 2-((6-(4-Fluorophenyl)pyridin-3-yl)oxy)-4-methyl-5-nitropyridine

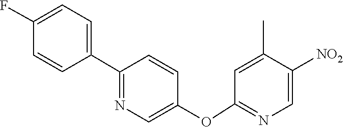 [SMILES: Cc1cc(Oc2ccc(-c3ccc(F)cc3)nc2)ncc1[N+](=O)[O-]]

Following the General procedure A, 2-((6-(4-fluorophenyl)pyridin-3-yl)oxy)-4-methyl-5-nitropyridin was obtained in 90% yield (2.70 mmol, 900 mg) from 6-(4-fluorophenyl)pyridin-3-01 (3.00 mmol, 600 mg; expl. 33, Step 3) and 2-chloro-4-methyl-5-nitropyridine (5.00 mmol, 800 mg).

C₁₇H₁₂FN₃O₃; Mw=325.29 g mol⁻¹; H NMR (400 MHz, CDCl₃) δ 8.86 (s, 1H), 8.55 (dd, J=2.8, 0.6 Hz, 1H), 8.01-7.95 (m, 2H), 7.76 (dd, J=8.7, 0.6 Hz, 1H), 7.60 (dd, J=8.6, 2.8 Hz, 1H), 7.17 (app t, J=8.8 Hz, 2H), 6.97 (s, 1H), 2.72 (s, 3H).

### Step 2: 6-((6-(4-Fluorophenyl)pyridin-3-yl)oxy)-4-methylpyridin-3-amine

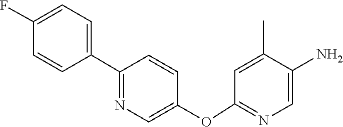 [SMILES: Cc1cc(Oc2ccc(-c3ccc(F)cc3)nc2)ncc1N]

Following the General procedure B, 6-((6-(4-fluorophenyl)pyridin-3-yl)oxy)-4-methylpyridin-3-amine was obtained in 96% yield (2.58 mmol, 763 mg) from 2-((6-(4-fluorophenyl)pyridin-3-yl)oxy)-4-methyl-5-nitropyridine (2.70 mmol, 900 mg).

C₁₇H₁₄FN₃O; Mw=295.31 g mol⁻¹; ¹H NMR (400 MHz, CDCl₃) δ 8.48 (dd, J=2.8, 0.6 Hz, 1H), 7.95-7.89 (m, 2H),7.66 (dd, J=8.1, 0.6 Hz, 1H), 7.64 (s, 1H), 7.47 (dd, J=8.7, 2.8 Hz, 1H), 7.19 (app t, J=8.7 Hz, 2H), 6.77 (s, 1H), 3.48 (s, 2H), 2.22 (s, 3H).

### BIOLOGICAL PROPERTIES OF COMPOUNDS

Identification of Novel Compounds to Target Oncovirus Positive and Driven Human Cancers

In order to identify novel compounds with an ability to block growth of oncovirus driven human cancers, the two EBV positive cell lines LCL070903 and HG-3 (Rosén A, Bergh AC, Gogok P, Evaldsson C, Myhrinder A L, Hellqvist E, Rasul A, Bjorkholm M, Jansson M, Mansouri L, Liu A, Teh B T, Rosenquist R, Klein E. Lymphoblastoid cell line with B1 cell characteristics established from a chronic lymphocytic leukemia clone by in vitro EBV infection Oncoimmunology. 2012 Jan. 1; 1(1):18-27) were used as representative cell lines. A set of compounds were tested for their ability to block growth and downregulate EBV target (cellular and viral) genes as described in following sections.

### Materials and Methods

Cell Culture

One million human EBV positive lymphoma cell lines HG3 and LCL070903 (Rosén et al., 2012) were cultured in RPMI1640 media supplemented with 10% FCS. For RNA expression analysis cells were treated with compounds at 10 μM concentration and for proliferation assay cells were treated at a concentration range of 0.01-100 μM. Following treatment cells were harvested and washed with 1× PBS. Total RNA was extracted as described below.

RNA Extraction

Total RNA was extracted from cells using TRIzol® extraction kit (Invitrogen). Briefly, 1×10⁶ cells were washed with ice-cold 1× PBS and lysed in 1 ml of TRIzol® solution for 5 minutes at room temperature to dissociate nucleoprotein complexes. Lysed cells were then treated with 200 μl of chloroform and shaked vigorously for 15-30 seconds and incubated at room temperature for 2-3 minutes. The samples were centrifuged at 14000 rpm using Eppendorf table top centrifuge for 10 minutes at 4° C. Following centrifugation, upper aqueous phase was transferred to new eppendorf tubes. To precipitate total RNA 500 μl of isomyl alcohol was added to the separated aqueous phase and incubated at room temperature for 10 minutes. A RNA pellet was obtained by centrifuging the samples at 4° C. for 10 minutes. RNA pellet obtained was washed with 1 ml ice cold 75% ethanol and spun down at 14000 rpm at 4° C. RNA pellet was dried off of excess of ethanol and resuspended in 40 _{I}A DPEC water.

cDNA Synthesis

Total RNA extracted from the cell was used to synthesize cDNA by reverse transcription reaction. Reverse transcription was performed according to one of the two following protocols.

In first protocol, SuperScript™ RT (Invitrogen) was used for reverse transcription reaction. RNA concentration was measured using NanoDrop®ND-1000 spectrophotometer (Witec AG) and 500 ng of total RNA was mixed with a 10 mM mix of dNTPs and 100 ng of random primers. The reaction mix was incubated at 65° C. for 5 minutes and quickly incubated on ice for 1 minute. Following incubation on ice, 5× first strand buffer and 0.1M DTT were added and mix was incubated for 2 minutes at 25° C. To start the reverse transcription reaction, 200U of SuperScrip™ II RT was added to the reaction mix and incubated at 42° C. for 50 minutes. The reaction was stopped by incubating the reaction mix at 75° C. for 15 minutes.

In second protocol, reverse transcription was performed using PrimeScript RT Master Mix (Takara). RNA concentration was measured using NanoDrop®ND-1000 spectrophotometer (Witec AG) and 1 μg of total RNA was mixed with 4 μL 5X PrimeScript RT Master Mix in a total reaction volume of 20 μL. The reaction mix was incubated at 37° C. for 15 minutes followed by heat inactivation at 85° C. for 5 seconds.

Quantitative Real Time PCR Analyses

QRT-PCR was carried out using 7900 HT Fast Real-Time PCR system (Applied Biosystems) or QuantStudio 3 system (ThermoFisher). Briefly, 12.5 ng of template cDNA was used with a primer concentration of 0.5 μM each and lx SYBR Green dye in a final volume of 10 μL in a 96 well or 384 well plate format.

Alamarblue/Prestoblue Proliferation Assay

Alamarblue® and PrestoBlue proliferation assays were performed to determine the growth kinetics of EBV inhibitor treated cells. Alamar blue® and PrestoBlue consists of a cell permeable substrate resazurin. In metabolically active and proliferating cells, resazurin is converted to resorufin due to an intrinsic reducing power of live cells and produces a red fluorescence. Therefore production of resorufin serves as an indicator of the viability of the cell population.

Proliferation assays were performed by seeding 5000 cells/well in a 96 well plate. Cells were treated with DMSO or compounds for 72 hours using concentration ranges of 0.01-100 μM. Each concentration was tested in 4 replicates. To determine the growth kinetics, 10 μl of Alamar blue® or PrestoBlue (Invitrogen) was added to each well and incubated for 4 hours. Readout was taken using Tecan F500 (Tecan) multiplate reader or Varioskan LUX (ThermoFisher) multiplate reader.

### Example 11

### Compounds Block Proliferation of EBV Positive Human Cancer Cells

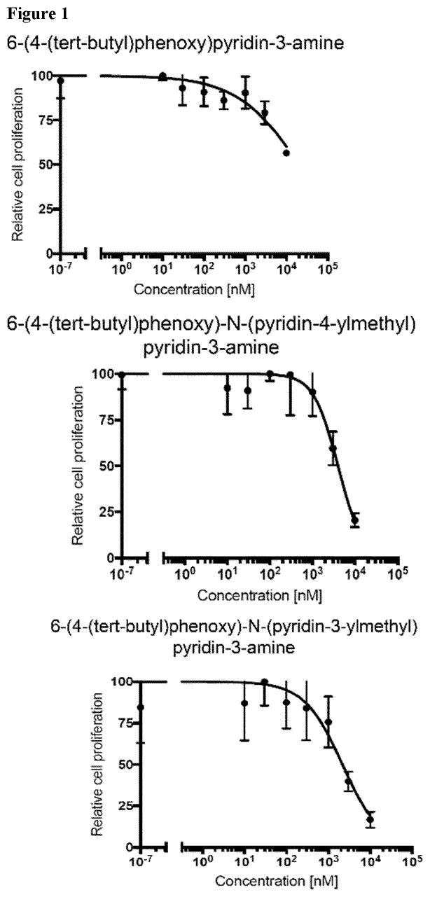

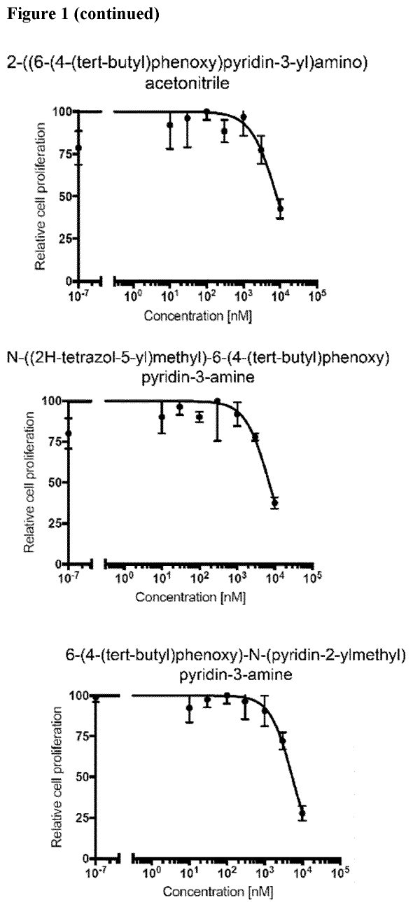

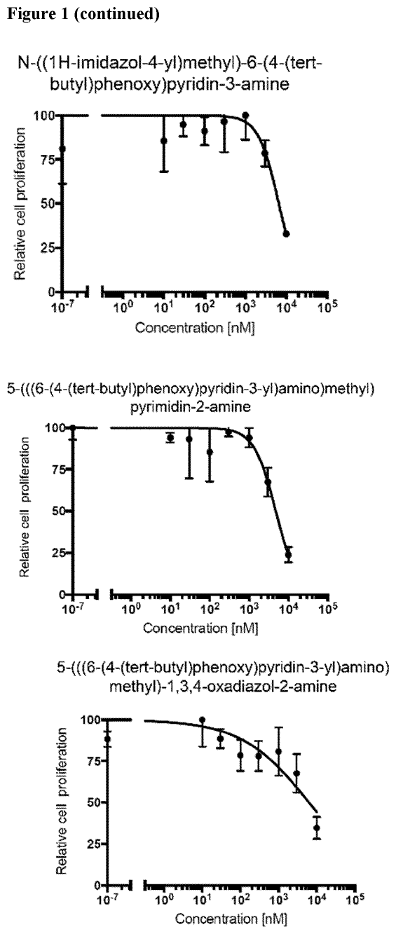

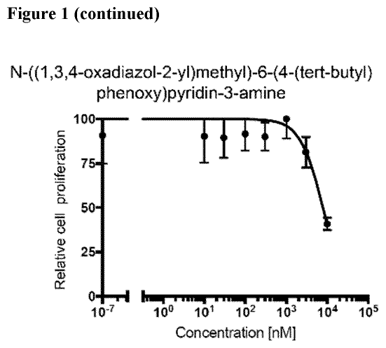

In order to determine anti-cancer activity of compounds in oncoviral driven cells, an EBV infected human Chronic Lymphocytic Leukemia cell line HG-3 was used (Rosen et al., 2012). In brief, HG-3 cells were plated in a 96 well plate and treated with an increasing concentration of compounds. As shown in FIG. 1 and table 1, 6-(4-(tert-Butyl)phenoxy)-N-(pyridin-4-ylmethyl)pyridin-3-amine, 6-(4-(tert-Butyl)phenoxy)-N-(pyridin-3-ylmethyl)pyridin-3-amine, 2-((6-(4-(tert-Butyl)phenoxy)pyridin-3-yl)amino)acetonitrile, N-((2H-Tetrazol-5-yl)methyl)-6-(4-(tert-butyl)phenoxy)pyridin-3-amine, 6-(4-(tert-Butyl)phenoxy)-N-(pyridin-2-ylmethyl)pyridin-3-amine, N-((1H-Imidazol-4-yl)methyl)-6-(4-(tert-butyl)phenoxy)pyridin-3-amine, 5-(((6-(4-(tert-Butyl)phenoxy)pyridin-3-yl)amino)methyl)pyrimidin-2-amine, 5-(((6-(4-(tert-Butyl)phenoxy)pyridin-3-yl)amino)methyl)-1,3,4-oxadiazol-2-amine and N-((1,3,4-Oxadiazol-2-yl)methyl)-6-(4-(tert-butyl)phenoxy)pyridin-3-amine demonstrate anti-proliferative effect on HG-3 cells. Moreover, the above mentioned compounds exhibit an enhanced potency compared to compound 6-(4-tert-Butylphenoxy)pyridin-3-amine described in WO 2013/093885.

TABLE 1
Anti-proliferative effect of compounds EBV positive human
lymphoma HG-3 cells.

| | Anti-proliferative IC₅₀ |
|---|---|
| Compounds | values (μM) |
| 6-(4-tert-Butylphenoxy)pyridin-3-amine* | >10 (outside the tested |
| | concentration range) |
| 6-(4-(tert-Butyl)phenoxy)-N- | 4.0 |
| (pyridin-4-ylmethyl)pyridin-3-amine | |
| 6-(4-(tert-Butyl)phenoxy)-N- | 2.2 |
| (pyridin-3-ylmethyl)pyridin-3-amine | |
| 2-((6-(4-(tert-Butyl)phenoxy) | 8 |
| pyridin-3-yl)amino)acetonitrile | |
| N-((2H-Tetrazol-5-yl)methyl)-6- | 7.1 |
| (4-(tert-butyl)phenoxy)pyridin-3-amine | |
| 6-(4-(tert-Butyl)phenoxy)-N- | 5.4 |
| (pyridin-2-ylmethyl)pyridin-3-amine | |
| N-((1H-Imidazol-4-yl)methyl)-6- | 6.6 |
| (4-(tert-butyl)phenoxy)pyridin-3-amine | |
| 5-(((6-(4-(tert-Butyl)phenoxy)pyridin-3-yl) | 4.8 |
| amino)methyl)pyrimidin-2-amine | |
| 5-(((6-(4-(tert-Butyl)phenoxy)pyridin-3-yl) | 6.3 |
| amino)methyl)-1,3,4-oxadiazol-2-amine | |
| N-((1,3,4-Oxadiazol-2-yl)methyl)- | 7.9 |
| 6-(4-(tert-butyl)phenoxy)pyridin-3-amine | |

*Comparative compound described in WO2013/093885
Cells were treated with compounds (concentration range 0.01-10 μM) for 72 hours. Anti-proliferative effect was measured using Alamar blue assay (See Material and methods for details). IC₅₀ values were calculated using Graph prism software. Data shows that compared to 6-(4-tert-Butylphenoxy)pyridin-3-amine, compounds 6-(4-(tert-Butyl)phenoxy)-N-(pyridin-4-ylmethyl)pyridin-3-amine, 6-(4-(tert-Butyl)phenoxy)-N-(pyridin-3-ylmethyl)pyridin-3-amine, 6-(4-(tert-Butyl)phenoxy)-N-(pyridin-2-ylmethyl)pyridin-3-amine, N-((1H-Imidazol-4-yl)methyl)-6-(4-(tert-butyl)phenoxy)pyridin-3-amine and 5-(((6-(4-(tert-Butyl)phenoxy)pyridin-3-yl)amino)methyl)pyrimidin-2-amine, 5-(((6-(4-(tert-Butyl)phenoxy)pyridin-3-yl)amino)methyl)-1,3,4-oxadiazol-2-amine and N-((1,3,4-Oxadiazol-2-yl)methyl)-6-(4-(tert-butyl)phenoxy)pyridin-3-amine block proliferation of EBV positive human cancer cells.

### Example 12

### Downregulation of EBV Target Genes

Upon infection of human cells, EBV is known to induce cancerous transformation of cells via upregulation of host as well as viral genes (e.g LMP1, RUNX3, EBNA2, BATF1 and CD21).

To determine the ability of compounds to downregulate EBV driven gene expression, HG-3 cells were treated with selected compounds and percentage inhibition of EBV target genes was determined by quantitative PCR. As shown in Table 2, compounds down regulate EBV target genes such as LMP1, RUNX3, EBNA2, BATF1 and CD21. To further determine the anti-oncoviral specificity of compounds, these compounds were tested for their ability to downregulate a NOTCH target gene HES1 in a NOTCH1 positive (EBV negative) human leukemic cell line RPMI8402. As shown in table 3, while a comparative compound 6-(4-tert-Butylphenoxy)pyridin-3-amine described in WO 2013/093885 effectively downregulates HES1, compounds 6-(4-(tert-Butyl)phenoxy)-N-(pyridin-4-ylmethyl)pyridin-3-amine, 6-(4-(tert-Butyl)phenoxy)-N-(pyridin-3-ylmethyl)pyridin-3-amine, 6-(4-(tert-Butyl)phenoxy)-N-(pyridin-2-ylmethyl)pyridin-3-amine), N-((1H-Imidazol-4-yl)methyl)-6-(4-(tert-butyl)phenoxy)pyridin-3-amine and 5-(((6-(4-(tert-Butyl)phenoxy)pyridin-3-yl)amino)methyl)pyrimidin-2-amine do not modulate expression of this gene. This data confirms specificity of these compounds in targeting EBV driven tumors.

TABLE 2
Effect of compounds on EBV target genes in
human HG-3 lymphoma cells.

| | EBV target gene (viral and cellular targets) | | | | |
|---|---|---|---|---|---|
| | downregulation (% inhibition) | | | | |
| Compounds | LMP1 | RUNX3 | EBNA2 | BATF1 | CD21 |
| 6-(4-(tert-Butyl) | 30 | 27 | ND | ND | ND |
| phenoxy)-N-(pyridin-4- | | | | | |
| ylmethyl)pyridin-3-amine | | | | | |
| 6-(4-(tert-Butyl) | ND | ND | ND | >90 | >90 |
| phenoxy)-N-(pyridin-3- | | | | | |
| ylmethyl)pyridin-3-amine | | | | | |
| 6-(4-(tert-Butyl) | 37 | 55 | 16 | ND | ND |
| phenoxy)-N-(pyridin-2- | | | | | |
| ylmethyl)pyridin-3-amine | | | | | |
| N-((1H-Imidazol-4-yl) | 15 | 37 | ND | ND | ND |
| methyl)-6-(4-(tert-butyl) | | | | | |
| phenoxy)pyridin-3-amine | | | | | |
| 5-(((6-(4-(tert-Butyl) | 22 | 57 | 46 | ND | ND |
| phenoxy)pyridin-3-yl) | | | | | |
| amino)methyl)pyrimidin- | | | | | |
| 2-amine | | | | | |

Percentage inhibition of EBV target genes achieved following treatment of cells with compounds and mRNA expression quantified by qPCR.
ND: not determined.

TABLE 3
Effect of compounds on NOTCH target gene HES1 in
RPMI 8402 cells.

| | Percentage inhibition of |
|---|---|
| Compounds | NOTCH target gene HES1 |
| 6-(4-tert-Butylphenoxy) | 80% |
| pyridin-3-amine* | |
| 6-(4-(tert-Butyl)phenoxy)- | 0% |
| N-(pyridin-4-ylmethyl) | |
| pyridin-3-amine | |
| 6-(4-(tert-Butyl)phenoxy)- | 0% |
| N-(pyridin-3-ylmethyl) | |
| pyridin-3-amine | |
| 6-(4-(tert-Butyl)phenoxy)- | 0% |
| N-(pyridin-2-ylmethyl) | |
| pyridin-3-amine | |
| N-((1H-Imidazol-4-yl) | 0% |
| methyl)-6-(4-(tert-butyl) | |
| phenoxy)pyridin-3-amine | |
| 5-(((6-(4-(tert-Butyl) | 0% |
| phenoxy)pyridin-3-yl) | |
| amino)methyl)pyrimidin- | |
| 2-amine | |

*Comparative compound described in WO2013/093885
NOTCH inhibitor 6-(4-tert-Butylphenoxy)pyridin-3-amine downregulates NOTCH target gene HES1 by about 80%. However compounds 6-(4-(tert-Butyl)phenoxy)-N-(pyridin-4-ylmethyl)pyridin-3-amine, 6-(4-(tert-Butyl)phenoxy)-N-(pyridin-3-ylmethyl)pyridin-3-amine, 6-(4-(tert-Butyl)phenoxy)-N-(pyridin-2-ylmethyl)pyridin-3-amine, N-((1H-Imidazol-4-yl)methyl-6-(4-(tert-butyl)phenoxy)pyridin-3-amine and 5-(((6-(4-(tert-Butyl)phenoxy)pyridin-3-yl)amino)methyl)pyrimidin-2-amine did not show any downregulation of the NOTCH pathway target gene HES1.

### Example 13

### Compounds Block Proliferation of Ebv Positive Human Cancer Cells

In order to determine anti-cancer activity of compounds in oncoviral driven cells, EBV infected human Chronic Lymphocytic Leukemia cell line HG-3 was used (Rosen et al., 2012).

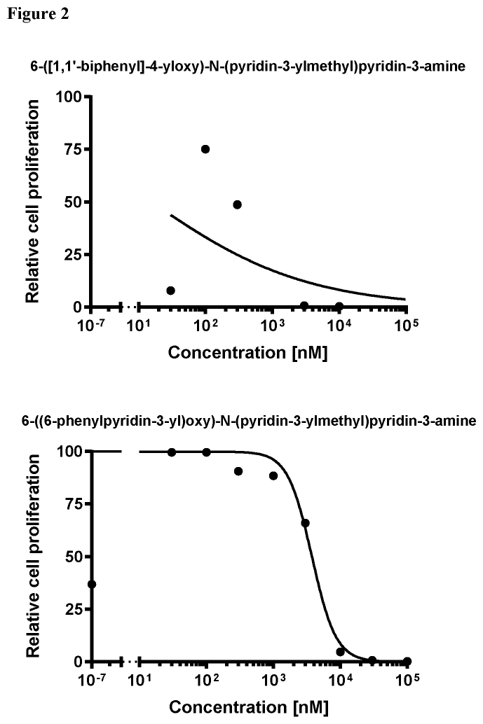

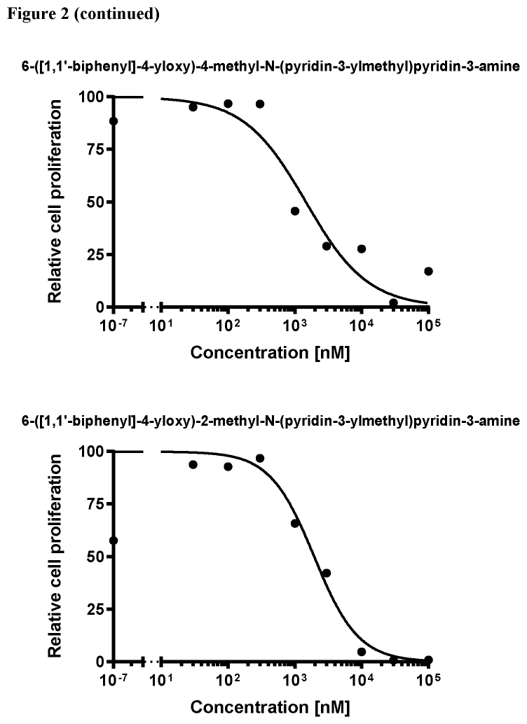

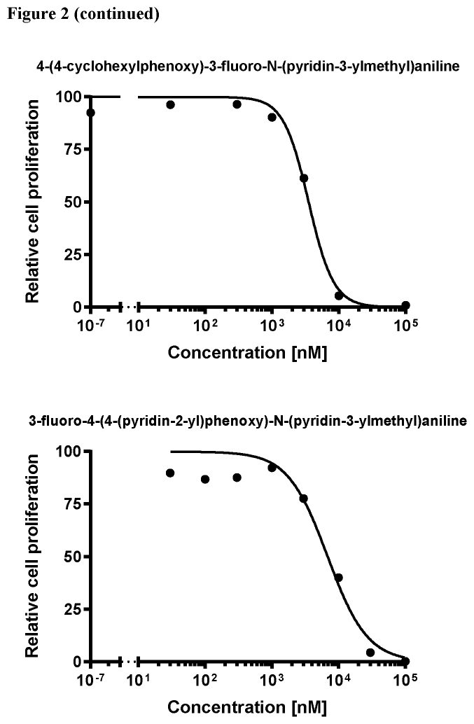

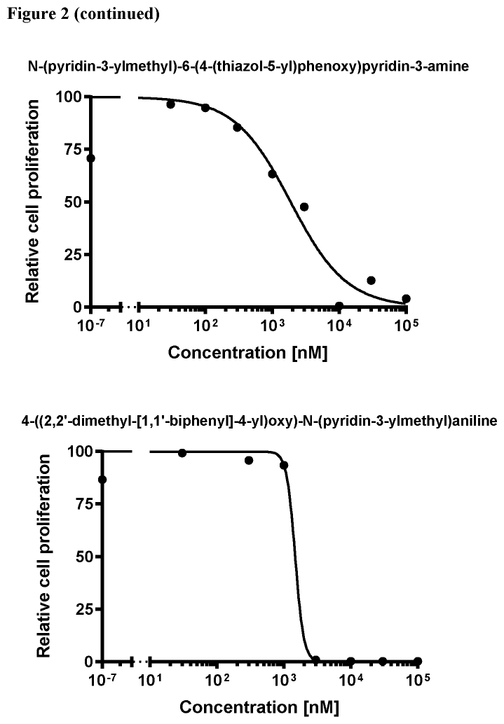

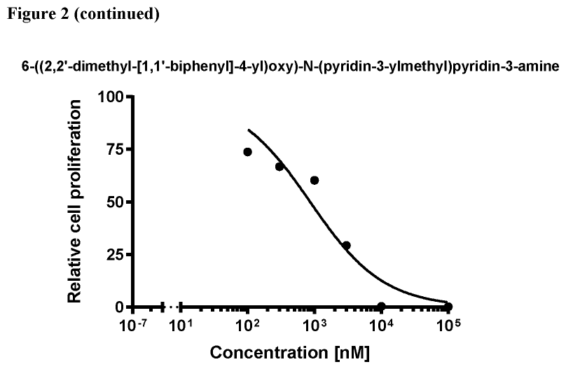

In brief, HG-3 cells were plated in a 96 well plate and treated with an increasing concentration of compounds. As shown in FIG. 2 and table 4, 6-([1,1′-biphenyl]-4-yloxy)-N-(pyridin-3-ylmethyl)pyridin-3-amine, 6-((6-phenylpyridin-3-yl)oxy)-N-(pyridin-3-ylmethyl)pyridin-3-amine, 6-([1,1′-biphenyl]-4-yloxy)-4-methyl-N-(pyridin-3-ylmethyl)pyridin-3-amine, 6-([1,1′-biphenyl]-4-yloxy)-2-methyl-N-(pyridin-3-ylmethyl)pyridin-3-amine, 4-(4-cyclohexylphenoxy)-3-fluoro-N-(pyridin-3-ylmethyl)aniline, 3-fluoro-4-(4-(pyridin-2-yl)phenoxy)-N-(pyridin-3-ylmethyl)aniline, N-(pyridin-3-ylmethyl)-6-(4-(thiazol-5-yl)phenoxy)pyridin-3-amine, 4-((2,2′-dimethyl-[1,1′-biphenyl]-4-yl)oxy)-N-(pyridin-3-ylmethyl)aniline and 6-((2,2′-dimethyl-[1,1′-biphenyl]-4-yl)oxy)-N-(pyridin-3-ylmethyl)pyridin-3-amine demonstrate anti-proliferative effect on HG-3 cells. Moreover, the above mentioned compounds exhibit an enhanced potency compared to compound 6-(4-tert-Butylphenoxy)pyridin-3-amine described in WO2013/093885.

TABLE 4
Anti-proliferative effect of compounds on EBV
positive human B HG-3 cells.

| | Anti-proliferative |
|---|---|
| | IC₅₀ values (μM) |
| Compounds | in HG-3 cells |
| 6-(4-tert-Butylphenoxy)pyridin-3-amine* | >10 (outside the tested |
| | concentration range) |
| 6-[(1,1′-biphenyl]-4-yloxy)-N-(pyridin- | 0.3 |
| 3-ylmethyl)pyridin-3-amine | |
| 6-((6-phenylpyridin-3-yl)oxy)-N- | 3.8 |
| (pyridin-3-ylmethyl)pyridin-3-amine | |
| 6-([1,1′-biphenyl]-4-yloxy)-4-methyl-N- | 1.5 |
| (pyridin-3-ylmethyl)pyridin-3-amine | |
| 6([1,1′-biphenyl]-4-yloxy)-2-methyl-N- | 1.9 |
| (pyridin-3-ylmethyl)pyridin-3-amine | |
| 4-(4-cyclohexylphenoxy)-3-fluoro-N- | 3.5 |
| (pyridin-3-ylmethyl)aniline | |
| 3-fluoro-4-(4-(pyridin-2-yl)phenoxy)- | 6.7 |
| N-(pyridin-3-ylmethyl)aniline | |
| N-(pyridin-3-ylmethyl)-6-(4-(thiazol- | 1.9 |
| 5-yl)phenoxy)pyridin-3-amine | |
| 4-((2,2′-dimethyl-[1,1′-biphenyl]-4-yl) | 1.5 |
| oxy)-N-(pyridin-3-ylmethyl)aniline | |
| 6-((2,2′-dimethyl-[1,1′-biphenyl]-4-yl) | 2.0 |
| oxy)-N-(pyridin-3-ylmethyl) | |
| pyridin-3-amine | |

*Comparative compound described in WO2013/093885
Cells were treated with compounds (concentration range 0.03-100 μM) for 72 hours. Anti-proliferative effect was measured using PrestoBlue assay (See Material and methods for details). IC₅₀ values were calculated using Graph prism software. Data shows that compared to 6-(4-tert-Butylphenoxy)pyridin-3-amine, compounds 6-([1,1′-biphenyl]-4-yloxy)-N-(pyridin-3-ylmethyl)pyridin-3-amine, 6-((6-phenylpyridin-3-yl)oxy)-N-(pyridin-3-ylmethyl)pyridin-3-amine, 6-([1,1′-biphenyl]-4-yloxy)-4-methyl-N-(pyridin-3-ylmethyl)pyridin-3-amine, 6-([1,1′-biphenyl]-4-yloxy)-2-methyl-N-(pyridin-3-ylmethyl)pyridin-3-amine, (4-(4-cyclohexylphenoxy)-3-fluoro-N-(pyridin-3-ylmethyl)aniline, 3-fluoro-4-(4-(pyridin-2-yl)phenoxy)-N-(pyridin-3-ylmethyl)aniline, N-(pyridin-3-ylmethyl)-6-(4-(thiazol-5-yl)phenoxy)pyridin-3-amine, 4-((2,2′-dimethyl-[1,1′-biphenyl]-4-yl)oxy)-N-(pyridin-3-ylmethyl)aniline and 6-((2,2′-dimethyl-[1,1′-biphenyl]-4-yl)oxy)-N-(pyridin-3-ylmethyl)pyridin-3-amine block proliferation of EBV positive human cancer cells.

### Example 14

### Downregulation of EBV Target Genes

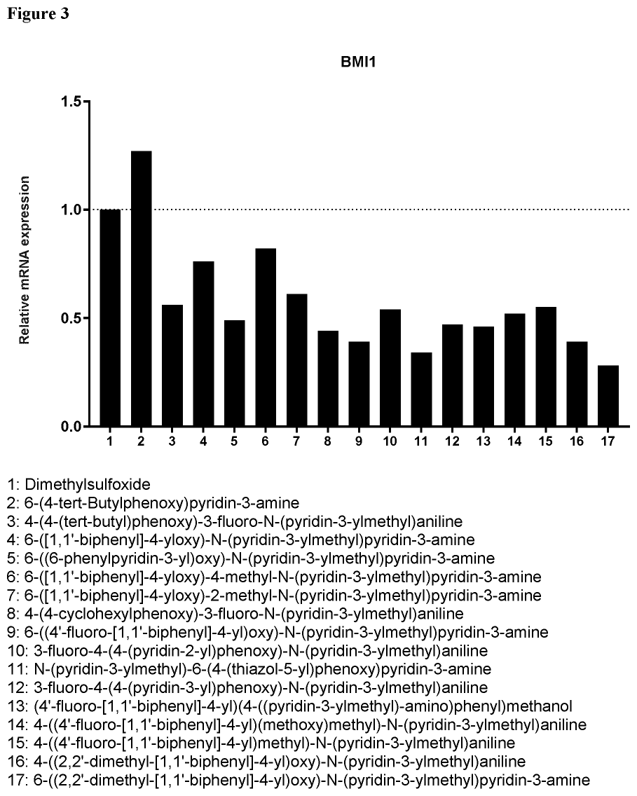

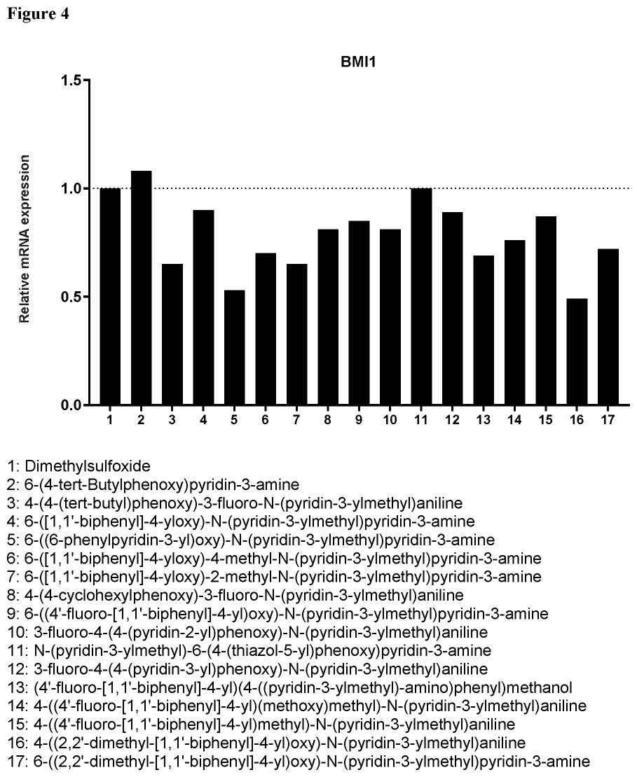

Upon infection of human cells, EBV is known to induce cancerous transformation of cells via upregulation of host as well as viral genes (e.g LMP1, EBNA2, BATF1, BMI1 and CD21). To determine the ability of compounds to downregulate EBV driven gene expression, HG-3 and LCL070903 cells were treated with selected compounds and percentage inhibition of EBV target genes was determined by quantitative PCR. As shown in Table 5 and Table 6, compounds down regulate EBV target genes such as BMI1 in HG-3 and LCL070903 cells, respectively (FIGS. 3 and 4).

TABLE 5
Effect of compounds on EBV target genes in
human HG-3 lymphoma cells.

| | EBV target gene | | | | |
|---|---|---|---|---|---|
| | (viral and cellular targets) | | | | |
| | downregulation (% inhibition) | | | | |
| Compounds | LMP1 | EBNA2 | BATF1 | BMI1 | CD21 |
| 6-(4-tert-Butylphenoxy) | N.D. | N.D. | N.D | Less | N.D. |
| pyridin-3-amine* | | | | than 0 | |
| 4-(4-(tert-butyl)phenoxy)-3- | N.D. | N.D. | N.D | 10 | N.D. |
| fluoro-N-(pyridin-3- | | | | | |
| ylmethyl)aniline | | | | | |
| 6-([1,1′-biphenyl]-4-yloxy)- | N.D. | N.D. | N.D. | 35 | N.D. |
| N-(pyridin-3-ylmethyl) | | | | | |
| pyridin-3-amine | | | | | |
| 6-((6-phenylpyridin-3-yl) | N.D. | N.D. | N.D | 47 | N.D. |
| oxy)-N-(pyridin-3-ylmethyl) | | | | | |
| pyridin-3-amine | | | | | |
| 6-([1,1′-biphenyl]-4-yloxy)- | N.D. | N.D. | N.D | 30 | N.D. |
| 4-methyl-N-(pyridin-3- | | | | | |
| ylmethyl)pyridin-3-amine | | | | | |
| 6-([1,1′-biphenyl]-4-yloxy)- | N.D. | 1 | N.D | 35 | N.D. |
| 2-methyl-N-(pyridin-3- | | | | | |
| ylmethyl)pyridin-3-amine | | | | | |
| 4-(4-cyclohexylphenoxy)- | N.D. | 36 | N.D | 19 | N.D. |
| 3-fluoro-N-(pyridin-3- | | | | | |
| ylmethyl)aniline | | | | | |
| 6-((4′-fluoro-[1,1′-biphenyl]- | N.D. | 7 | N.D. | 15 | N.D. |
| 4-yl)oxy)-N-(pyridin-3- | | | | | |
| ylmethyl)pyridin-3-amine | | | | | |
| 3-fluoro-4-(4-(pyridin-2- | N.D. | 1 | N.D. | 19 | N.D. |
| yl)phenoxy)-N-(pyridin-3- | | | | | |
| ylmethyl)aniline | | | | | |
| N-(pyridin-3-ylmethyl)-6- | N.D. | N.D. | N.D. | N.D. | N.D. |
| (4-(thiazol-5-yl)phenoxy) | | | | | |
| pyridin-3-amine | | | | | |
| 3-fluoro-4-(4-(pyridin-3- | N.D. | N.D. | N.D | 11 | N.D. |
| yl)phenoxy)-N-(pyridin-3- | | | | | |
| ylmethyl)aniline | | | | | |
| (4′-fluoro-[1,1′-biphenyl]-4- | N.D. | N.D. | N.D | 31 | 10 |
| yl)(4-((pyridin-3-ylmethyl)- | | | | | |
| amino)phenyl)methanol | | | | | |
| 4-((4′-fluoro-[1,1′-biphenyl]- | N.D. | N.D. | N.D | 24 | N.D. |
| 4-yl)(methoxy)methyl)-N- | | | | | |
| (pyridin-3-ylmethyl)aniline | | | | | |
| 4-((4′-fluoro-[1,1′-biphenyl]- | N.D. | N.D. | N.D | 13 | N.D. |
| 4-yl)methyl)-N-(pyridin-3- | | | | | |
| ylmethyl)aniline | | | | | |
| 4-((2,2′-dimethyl-[1,1′- | N.D. | 36 | N.D | 51 | N.D. |
| biphenyl]-4-yl)oxy)-N- | | | | | |
| (pyridin-3-ylmethyl)aniline | | | | | |
| 6-((2,2′-dimethyl-[1,1′- | N.D. | 6 | N.D | 28 | N.D. |
| biphenyl]-4-yl)oxy)-N- | | | | | |
| (pyridin-3-ylmethyl)pyridin- | | | | | |
| 3-amine | | | | | |

*Comparative compound described in WO2013/093885
Percentage inhibition of EBV target genes achieved following treatment of cells with compounds and mRNA expression quantified by qPCR.
ND: not determined.

TABLE 6
Effect of compounds on EBV target genes in human LCL070903 cells.

| | EBV target gene | | | | |
|---|---|---|---|---|---|
| | (viral and cellular targets) | | | | |
| | downregulation (% inhibition) | | | | |
| Compounds | LMP1 | EBNA2 | BATF1 | BMI1 | CD21 |
| 6-(4-tert-Butylphenoxy) | N.D. | N.D. | N.D | Less | N.D. |
| pyridin-3-amine* | | | | than 0 | |
| 4-(4-(tert-butyl)phenoxy)- | N.D. | N.D. | N.D | 24 | N.D. |
| 3-fluoro-N-(pyridin-3- | | | | | |
| ylmethyl)aniline | | | | | |
| 6-([1,1′-biphenyl]-4-yloxy)- | N.D. | 27 | N.D | 44 | N.D. |
| N-(pyridin-3-ylmethyl) | | | | | |
| pyridin-3-amine | | | | | |
| 6-((6-phenylpyridin-3-yl) | 12 | 50 | N.D | 51 | 9 |
| oxy)-N-(pyridin-3-ylmethyl) | | | | | |
| pyridin-3-amine | | | | | |
| 6-([1,1′-biphenyl]-4-yloxy)- | N.D. | N.D. | N.D. | 18 | N.D. |
| 4-methyl-N-(pyridin-3- | | | | | |
| ylmethyl)pyridin-3-amine | | | | | |
| 6-([1,1′-biphenyl]-4-yloxy)- | N.D. | 1 | N.D. | 39 | N.D. |
| 2-methyl-N-(pyridin-3- | | | | | |
| ylmethyl)pyridin-3-amine | | | | | |
| 4-(4-cyclohexylphenoxy)-3- | N.D. | 19 | N.D | 56 | N.D. |
| fluoro-N-(pyridin-3- | | | | | |
| ylmethyl)aniline | | | | | |
| 6-((4′-fluoro-[1,1′-biphenyl]- | N.D. | N.D. | N.D | 61 | N.D. |
| 4-yl)oxy)-N-(pyridin-3- | | | | | |
| ylmethyl)pyridin-3-amine | | | | | |
| 3-fluoro-4-(4-(pyridin-2-yl) | N.D. | N.D. | N.D | 46 | N.D. |
| phenoxy)-N-(pyridin-3- | | | | | |
| ylmethyl)aniline | | | | | |
| N-(pyridin-3-ylmethyl)-6- | N.D. | N.D. | N.D | 66 | 22 |
| (4-(thiazol-5-yl)phenoxy) | | | | | |
| pyridin-3-amine | | | | | |
| 3-fluoro-4-(4-(pyridin-3-yl) | N.D. | N.D. | N.D | 53 | N.D. |
| phenoxy)-N-(pyridin-3- | | | | | |
| ylmethyl)aniline | | | | | |
| (4′-fluoro-[1,1′-biphenyl]- | N.D. | N.D. | N.D | 54 | N.D. |
| 4-yl)(4-((pyridin-3-ylmethyl)- | | | | | |
| amino)phenyl)methanol | | | | | |
| 4-((4′-fluoro-[1,1′-biphenyl]- | N.D. | N.D. | N.D | 48 | N.D. |
| 4-yl)(methoxy)methyl)-N- | | | | | |
| (pyridin-3-ylmethyl)aniline | | | | | |
| 4-((4′-fluoro-[1,1′-biphenyl]- | N.D. | N.D. | N.D | 45 | N.D. |
| 4-yl)methyl)-N-(pyridin-3- | | | | | |
| ylmethyl)aniline | | | | | |
| 4-((2,2′-dimethyl-[1,1′- | 24 | 43 | N.D | 61 | 1 |
| biphenyl]-4-yl)oxy)-N- | | | | | |
| (pyridin-3-ylmethyl)aniline | | | | | |
| 6-((2,2′-dimethyl-[1,1′- | N.D. | 30 | N.D | 72 | N.D. |
| biphenyl]-4-yl)oxy)-N- | | | | | |
| (pyridin-3-ylmethyl)pyridin- | | | | | |
| 3-amine | | | | | |

*Comparative compound described in WO2013/093885
Percentage inhibition of EBV target genes achieved following treatment of cells with compounds and mRNA expression quantified by qPCR.
ND: not determined.

1. A compound of formula (I) 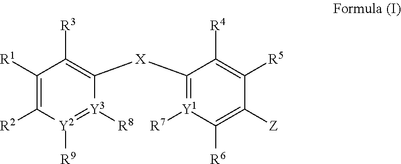
pharmaceutically-acceptable salts, hydrates, solvates, or stereoisomers thereof,
wherein X is selected from CH₂, CF₂, CHF, CO, CHOH, CHO(C₁-C₃) alkyl, NH, N(C₁-C₃ alkyl), S, SO and O;
wherein Y¹, Y² and Y³ are each independently selected from N and C;
wherein Z is NR¹⁰R¹¹;
wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and
R¹¹ is selected from C₁-C₃ cyanoalkyl, and C₁-C₆ alkyl substituted by aryl, heteroaryl,
C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³;
wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³; C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl;
wherein R² is selected from COC₁-C₆ alkyl, NH₂, OH, CN, SO₃H, S(O)ₙ(C₁-C₆ alkyl)
wherein n is 0, 1, or 2, C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₂- C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
wherein R³ is selected from H, halogen, C₁-C₆ alkyl, C₁-C₆ heteroalkyl, C₃-C₁₂ cycloalkyl and C₃-C₁₂ heterocyclyl;
wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆-S-alkyl, C₁-C₆ alkyl, C₁-C₆ heteroalkyl, C₃-C₁₂ cycloalkyl and C₃-C₁₂ heterocyclyl;
wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;
wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;
wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C¹⁻C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C; and
wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl.

2. The compound of formula (I) according to claim 1, wherein R¹ is selected from H, halogen, C₁-C₆ alkyl, C₃-C₁₂ cycloalkyl and C₁-C₆ alkoxy.

3. The compound of formula (I) according to anyone of claims 1-2, wherein R³ is selected from H, halogen, C₁-C₆ alkyl and C₁-C₆ heteroalkyl.

4. The compound of formula (I) according to anyone of claims 1-3, wherein R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl.

5. The compound of formula (I) according to anyone of claims 1-4, wherein R⁷ is absent when Y¹ is N or is selected from H, halogen, C₁-C₆ alkyl and C₁-C₆ heteroalkyl when Y¹ is C and/or wherein R⁸ is absent when Y³ is N or is selected from H, halogen, C₁-C₆ alkyl and C₁-C₆ heteroalkyl when Y³ is C and/or wherein R⁹ is absent when Y² is N or is selected from H, halogen, C₁-C₆ alkyl and C₁-C₆ heteroalkyl when Y² is C.

6. The compound of formula (I) according to anyone of claims 1-5, wherein R¹² and R¹³ are selected from H and C₁-C₆ alkyl.

7. The compound of formula (I) according to anyone of claims 1-6, wherein R¹⁰ is H and R¹¹ is selected from C₁-C₃ cyanoalkyl, and C₁-C₆ alkyl substituted by aryl or heteroaryl, preferably is C₁-C₆ alkyl substituted by aryl or heteroaryl, wherein the aryl and the heteroaryl is optionally substituted by NH₂, C₁-C₆ alkyl, C₁-C₆ heteroalkyl, halogen, CN, C₃-C₁₂ cycloalkyl, C₃-C₁₂ heterocyclyl.

8. A compound of formula (I) according to anyone of claims 1-6, wherein
R¹⁰ is H;
R¹¹ is selected from C₁-C₃ cyanoalkyl, and C₁-C₆ alkyl substituted by aryl, heteroaryl,
C₃-C₁₂ heterocyclyl, wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl,
C₁-C₆ alkyl, C₁-C₆ heteroalkyl, halogen, CN, C₃-C₁₂ cycloalkyl, C₃-C₁₂ heterocyclyl, C(O)NR¹², C(O)NR¹²R¹³; and
R² is selected from COC₁-C₆ alkyl, NH₂, OH, CN, SO₃H, S(O)ₙ(C₁-C₆ alkyl) wherein n is 0, 1 or 2, C₂-C₆ alkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C(O)NR¹²R¹³; C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl.

9. The compound of formula (I) according to anyone of claims 1-6, wherein
R¹⁰ is H;
R¹¹ is selected from C₁-C₃ cyanoalkyl, and C₁-C₆ alkyl substituted by aryl, heteroaryl,
C₃-C₁₂ heterocyclyl, wherein the heteroaryl is not 3H-imidazole-4-yl and wherein the aryl, the heteroaryl and the C₃-C₁₂ heterocyclyl are optionally substituted by NH₂, N(C₁-C₆ alkyl)₂, NH(C₁-C₆ alkyl), OH, O(C₁-C₆) alkyl, C₁-C₆ alkyl, C₁-C₆ heteroalkyl, halogen, CN, C₃-C₁₂ cycloalkyl, C₃-C₁₂ heterocyclyl, C(O)NR¹², C(O)N R¹²R¹³.

10. The compound of formula (I) according to claim 1, wherein
X is selected from CH₂, CF₂, CHF, CO, CHOH, CHO(C₁-C₃) alkyl, NH, N(C₁-C₃ alkyl), S, SO and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;
R¹ is selected from H, halogen, C₁-C₆ alkyl, C₃-C₁₂ cycloalkyl and C₁-C₆ alkoxy;
R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
R³ is selected from H, halogen, C₁-C₆ alkyl and C₁-C₆ heteroalkyl;
R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C;
R⁹ is absent when Y² is N or is selected from H, halogen and C₁-C₆ alkyl when Y² is C.

11. The compound of formula (I) according to claim 1, wherein
X is selected from CH₂, CF₂, CHF, CO, CHOH, CHO(C₁-C₃) alkyl, NH, N(C₁-C₃ alkyl), S, SO and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;
R¹ is selected from H, halogen, and C₁-C₆ alkyl;
R² is selected from C₂-C₆ alkyl, C₂-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C;
R⁹ is absent when Y² is N or is selected from H, halogen and C₁-C₆ alkyl when Y² is C.

12. The compound of formula (I) according to anyone of claims 1-11, wherein R² is selected from C₂-C₆ alkyl, C₂-C₆ heteroalkyl wherein the heterosubstituent is not halogen and is preferably selected from OH and NH₂, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl.

13. The compound of formula (I) according to anyone of claims 1-12, wherein
X is selected from CH₂, CO, CHOH, CHO(C₁-C₃) alkyl, NH and O.

14. The compound of formula (I) according to claim 1, wherein
X is selected from CH₂, CO, CHOH, CHO(C₁-C₃) alkyl, NH and O;
Y¹, Y², and Y³ are each independently selected from N and C;
Z is NR¹⁰R¹¹;
R¹⁰ is H;
R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C; and
R⁹ is absent when Y² is N or is selected from H, halogen and C₁-C₆ alkyl when Y² is C.

15. The compound of formula (I) according to claim 1, wherein
X is selected from CH₂, CO, CHOH, CHO(C₁-C₃) alkyl, NH and O;
Y¹ is selected from N and C, Y² is selected from N and C and Y³ is C;
Z is NR¹⁰R¹¹;
R¹⁰ is H;
R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is selected from H, halogen and C₁-C₆ alkyl; and
R⁹ is absent when Y² is N or is selected from H, halogen and C₁-C₆ alkyl when Y² is C.

16. The compound of formula (I) according to claim 1, wherein
X is selected from CH₂, CO, CHOH, CHO(C₁-C₃) alkyl, NH and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C; and
R⁹ is absent when Y² is N or is selected from H, halogen and C₁-C₆ alkyl when Y² is C.

17. The compound of formula (I) according to claim 1, wherein
X is selected from CH₂, CO, CHOH, CHO(C₁-C₃) alkyl, NH and O;
Y¹ is selected from N and C, Y² is selected from N and C and Y³ is C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is selected from H, halogen and C₁-C₆ alkyl; and
R⁹ is absent when Y² is N or is selected from H, halogen and C₁-C₆ alkyl when Y² is C.

18. The compound of formula (I) according to anyone of claims 1-17, wherein the heteroaryl group of R¹¹ is not 3H-imidazole-4-yl.

19. The compound of formula (I) according to anyone of claims 1-17, wherein the heteroaryl group of R¹¹ is not 3H-imidazole-4-yl and not 1H-imidazole-4-yl.

20. The compound of formula (I) according to claim 1, wherein
X is selected from CH₂, CO, CHOH, CHO(C₁-C₃) alkyl, NH and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C; and
R⁹ is absent when Y² is N or is selected from H, halogen and C₁-C₆ alkyl when Y² is C.

21. The compound of formula (I) according to claim 1, wherein
X is selected from CH₂, CO, CHOH, CHO(C₁-C₃) alkyl, NH and O;
Y¹ is selected from N and C, Y² is selected from N and C and Y³ is C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₂-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is selected from H, halogen and C₁-C₆ alkyl; and
R⁹ is absent when Y² is N or is selected from H, halogen and C₁-C₆ alkyl when Y² is C.

22. The compound of formula (I) according to anyone of claims 1-21, wherein
X is selected from CH₂, CHOH, CHO(C₁-C₃) alkyl, and O.

23. The compound of formula (I) according to anyone of claims 1-22, wherein X is O.

24. A compound according to anyone of claims 1-22 selected from the group consisting of: 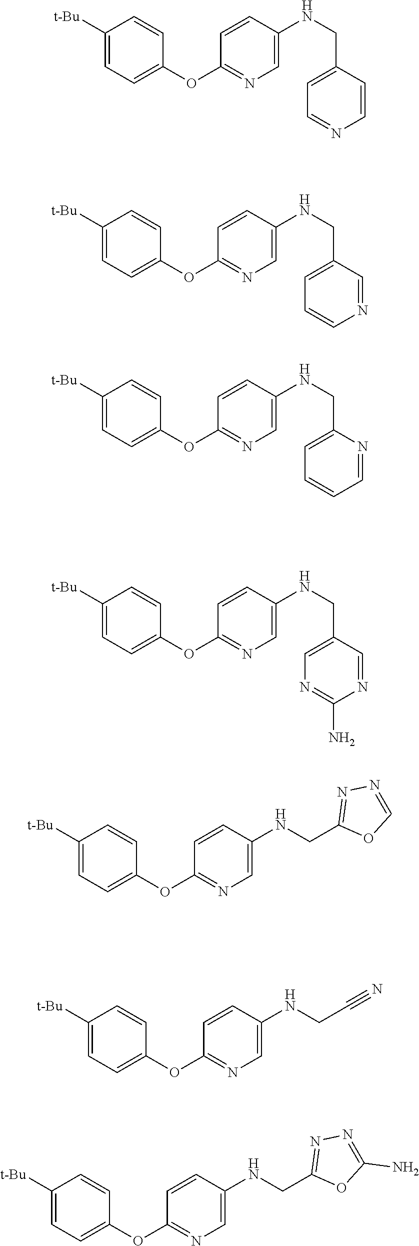 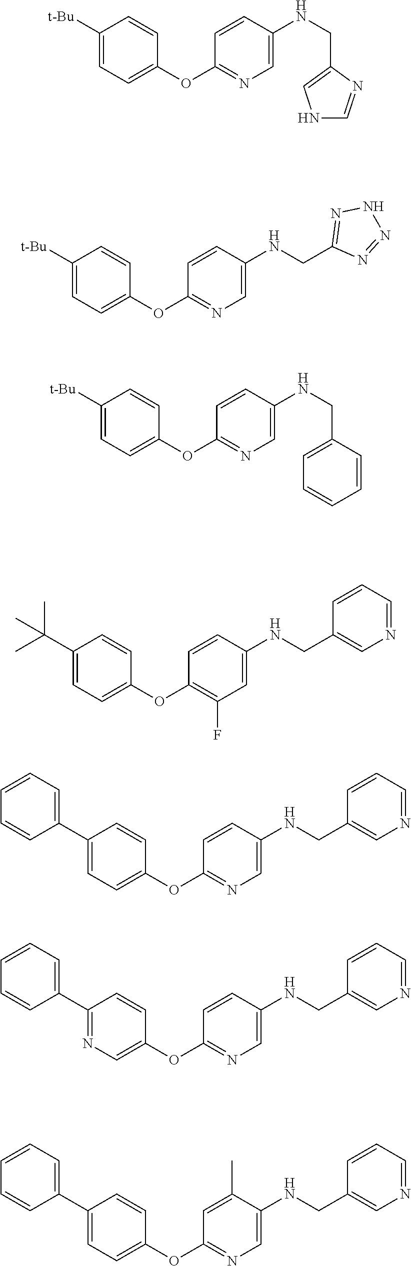 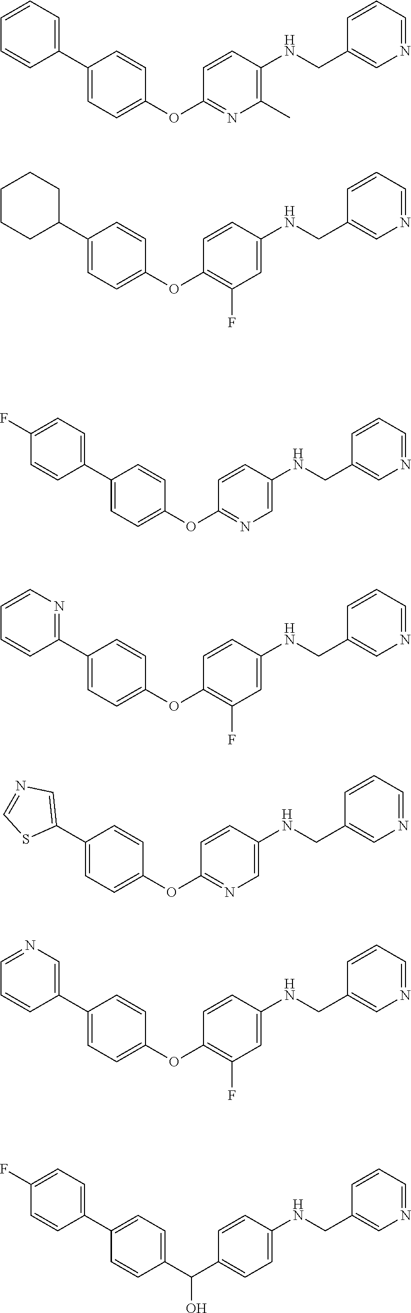 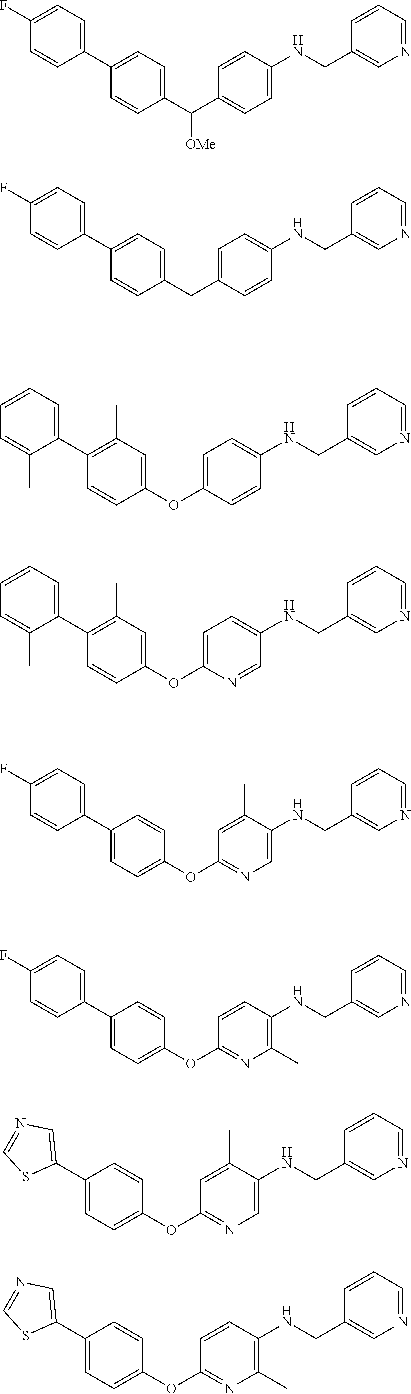 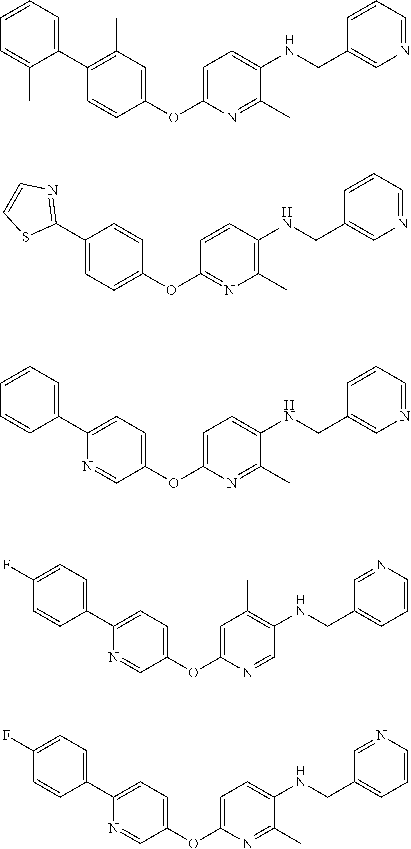 [SMILES: Cc1ccccc1-c1ccc(Oc2ccc(NCc3cccnc3)c(C)n2)cc1C ; Cc1nc(Oc2ccc(-c3nccs3)cc2)ccc1NCc1cccnc1 ; Cc1nc(Oc2ccc(-c3ccccc3)nc2)ccc1NCc1cccnc1 ; Cc1cc(Oc2ccc(-c3ccc(F)cc3)nc2)ncc1NCc1cccnc1 ; Cc1nc(Oc2ccc(-c3ccc(F)cc3)nc2)ccc1NCc1cccnc1]
and pharmaceutically-acceptable salts, hydrates, solvates, or stereoisomers thereof

25. A compound according to anyone of claims 1-22 selected from the group consisting of: 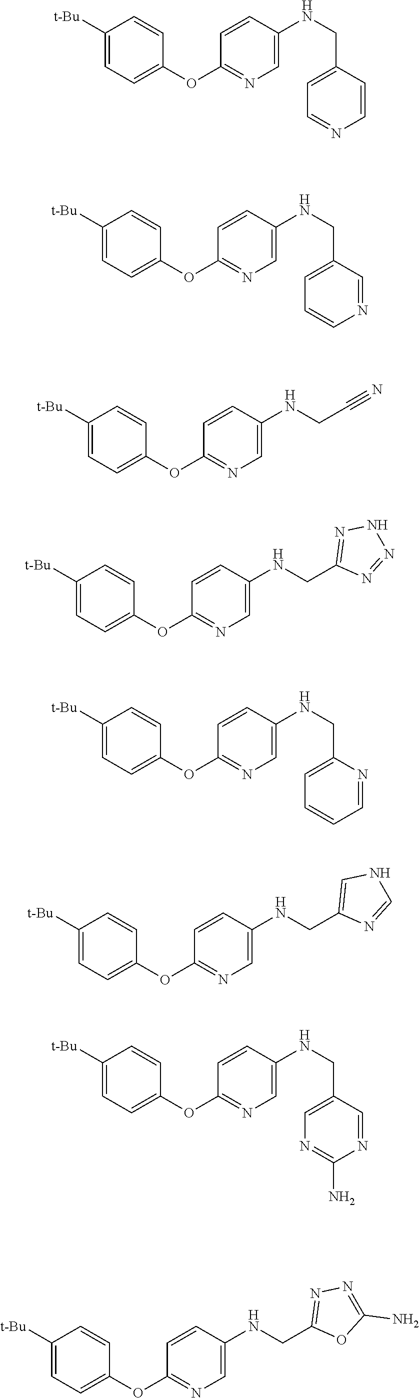 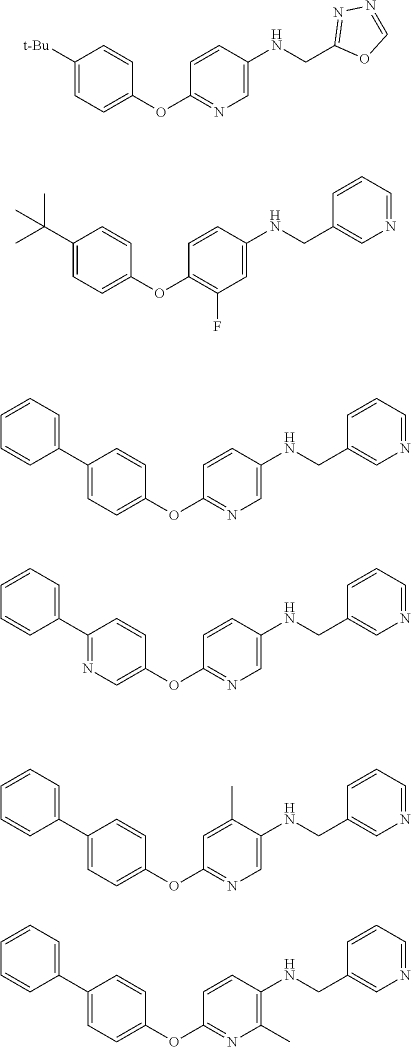 [SMILES: CC(C)(C)c1ccc(Oc2ccc(NCc3nnco3)cn2)cc1 ; CC(C)(C)c1ccc(Oc2ccc(NCc3cccnc3)cc2F)cc1 ; c1ccc(-c2ccc(Oc3ccc(NCc4cccnc4)cn3)cc2)cc1 ; c1ccc(-c2ccc(Oc3ccc(NCc4cccnc4)cn3)cn2)cc1 ; Cc1cc(Oc2ccc(-c3ccccc3)cc2)ncc1NCc1cccnc1 ; Cc1nc(Oc2ccc(-c3ccccc3)cc2)ccc1NCc1cccnc1] 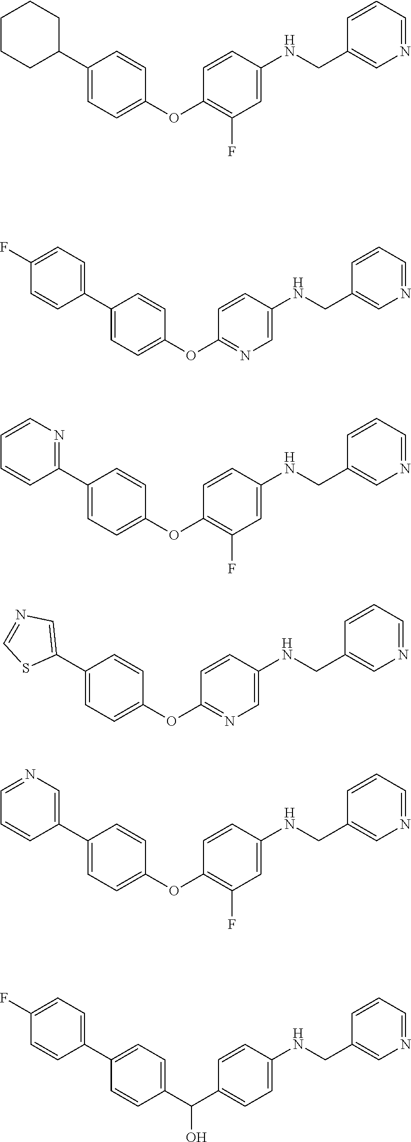 [SMILES: Fc1cc(NCc2cccnc2)ccc1Oc1ccc(C2CCCCC2)cc1 ; Fc1ccc(-c2ccc(Oc3ccc(NCc4cccnc4)cn3)cc2)cc1 ; Fc1cc(NCc2cccnc2)ccc1Oc1ccc(-c2ccccn2)cc1 ; c1cncc(CNc2ccc(Oc3ccc(-c4cncs4)cc3)nc2)c1 ; Fc1cc(NCc2cccnc2)ccc1Oc1ccc(-c2cccnc2)cc1 ; OC(c1ccc(NCc2cccnc2)cc1)c1ccc(-c2ccc(F)cc2)cc1] 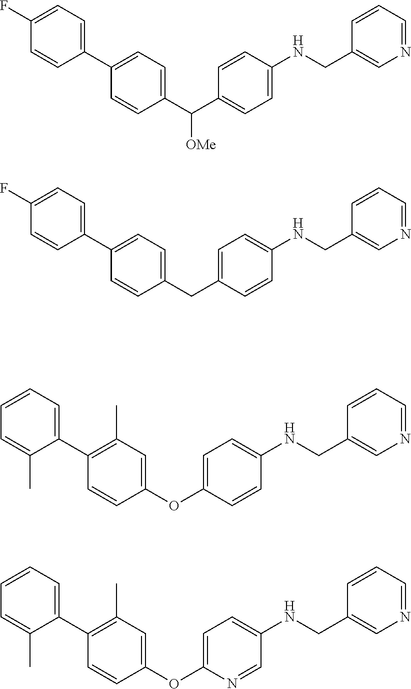 [SMILES: COC(c1ccc(NCc2cccnc2)cc1)c1ccc(-c2ccc(F)cc2)cc1 ; Fc1ccc(-c2ccc(Cc3ccc(NCc4cccnc4)cc3)cc2)cc1 ; Cc1ccccc1-c1ccc(Oc2ccc(NCc3cccnc3)cc2)cc1C ; Cc1ccccc1-c1ccc(Oc2ccc(NCc3cccnc3)cn2)cc1C]
and pharmaceutically-acceptable salts, hydrates, solvates, or stereoisomers thereof.

26. A compound according to anyone of claims 1-22 selected from the group consisting of: 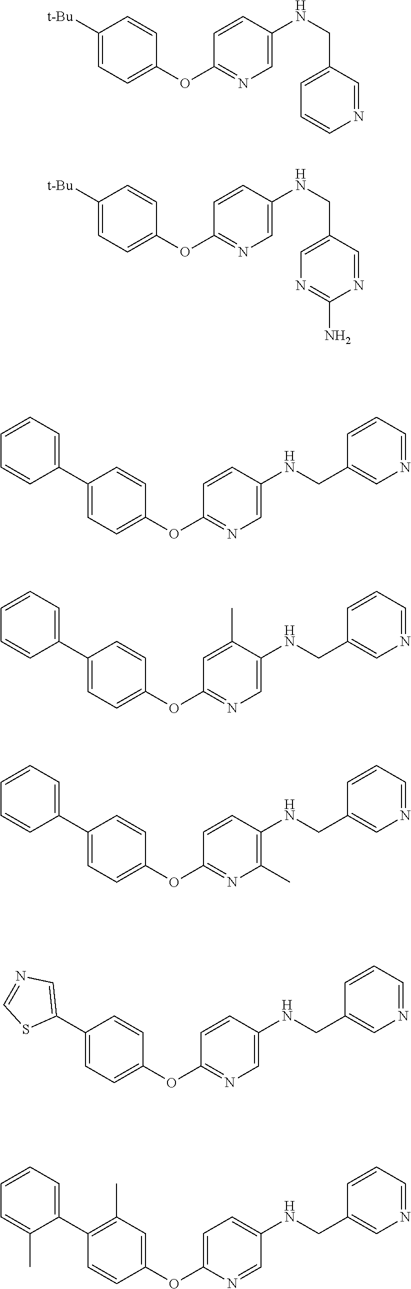 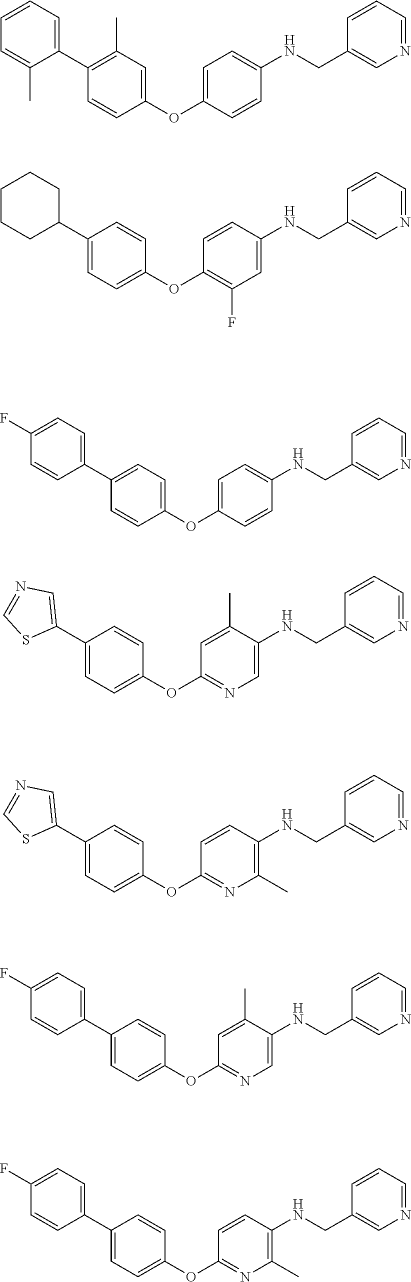 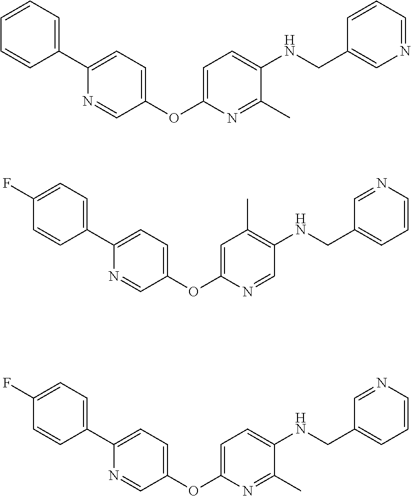 [SMILES: Cc1nc(Oc2ccc(-c3ccccc3)nc2)ccc1NCc1cccnc1 ; Cc1cc(Oc2ccc(-c3ccc(F)cc3)nc2)ncc1NCc1cccnc1 ; Cc1nc(Oc2ccc(-c3ccc(F)cc3)nc2)ccc1NCc1cccnc1]
and pharmaceutically-acceptable salts, hydrates, solvates, or stereoisomers thereof.

27. A compound of formula (I) 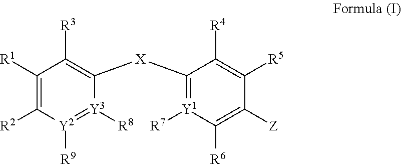
pharmaceutically-acceptable salts, hydrates, solvates, or stereoisomers thereof,
wherein X is selected from CH₂, CF₂, CHF, CO, CHOH, CHO(C₁-C₃) alkyl, NH, N(C₁-C₃ alkyl), S, SO and O;
wherein Y¹, Y² and Y³ are each independently selected from N and C;
wherein Z is NR¹⁰R¹¹;
wherein R¹⁰ is selected from H, C₁-C₆ alkyl, C₁-C₃ cyanoalkyl, and C₁-C₆ alkyl substituted by aryl, heteroaryl, C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl, C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³; and
R¹¹ is selected from C₁-C₃ cyanoalkyl, and C₁-C₆ alkyl substituted by aryl, heteroaryl,
C₃-C₁₂ heterocyclyl wherein the aryl, the heteroaryl and the C₃-C₁₂ heterocyclyl are optionally substituted by NH₂, NH(C₁-C₆ alkyl), N(C₁-C₆ alkyl)₂, OH, OC₁-C₆ alkyl,
C₁-C₆ alkyl, C₁-C₆ heteroalkyl, halogen, CN, C₃-C₁₂ cycloalkyl, C₃-C₁₂ heterocyclyl, C(O)OR¹², C(O)NR¹²R¹³;
wherein R¹ is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³;
C(O)NR¹²R¹³; C₁-C₆ alkyl-C(O)NR¹²R¹³; C₁-C₆ alkoxy, C₁-C₃ alkoxycarbonyl, C₁-C₆ alkyl-NHCOR¹², C₁-C₃ alkanoyl, adamantyl and norbornyl;
wherein R² is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl, norbornyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
wherein R³ is selected from H, halogen, C₁-C₆ alkyl, C₁-C₆ heteroalkyl, C₃-C₁₂ cycloalkyl and C₃-C₁₂ heterocyclyl;
wherein R⁴, R⁵ and R⁶ are each independently selected from H, OH, halogen, NH₂, NO₂, C₁-C₆ alkylamino, C₁-C₆ dialkylamino, C₁-C₆ alkoxy, C₁-C₆-S-alkyl, C₁-C₆ alkyl, C₁-C₆ heteroalkyl, C₃-C₁₂ cycloalkyl and C₃-C₁₂ heterocyclyl;
wherein R⁷ is absent when Y¹ is N or is selected from H, halogen, C₁-C₆ alkyl, C₁-C₆ heteroalkyl, C₃-C₁₂ cycloalkyl and C₃-C₁₂ heterocyclyl when Y¹ is C;
wherein R⁸ is absent when Y³ is N or is selected from H, halogen, C₁-C₆ alkyl, C₁-C₆ heteroalkyl, C₃-C₁₂ cycloalkyl and C₃-C₁₂ heterocyclyl when Y³ is C;
wherein R⁹ is absent when Y² is N or is selected from H, halogen, COC₁-C₆ alkyl, NH₂, OH, CN, SO₃H, S(O)ₙ(C₁-C₆ alkyl) wherein n is 0, 1, or 2, C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, C₁-C₆ alkylamino, C₁-C₆ dialkylamino, carboxy, C₁-C₆ alkyl-carboxy, C₁-C₃ alkyl-NHC(O)OR¹², C₁-C₃ alkyl-OC(O)NR¹²R¹³, C(O)NR¹²R¹³, C₁-C₆ alkyl-C(O)NR¹²R¹³, C₁-C₆ alkoxy, C₁-C₃ alkoxycarbonyl, C₁-C₆ alkyl-NHCOR¹², C₁-C₃ alkanoyl, adamantyl and norbornyl when Y² is C; and
wherein R¹² and R¹³ are each independently selected from H, C₁-C₆ alkyl, C₁-C₆ heteroalkyl, C₂-C₆ alkenyl, C₂-C₆ alkynyl, C₃-C₁₂ cycloalkyl and C₃-C₁₂ heterocyclyl for use in a method for the prevention or treatment of oncovirus induced cancer in a subject.

28. The compound of formula (I) for use according to claim 27, wherein R² is selected from C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl.

29. The compound of formula (I) for use according to claim 27, wherein
X is selected from CH₂, CF₂, CHF, CO, CHOH, CHO(C₁-C₃) alkyl, NH, N(C₁-C₃ alkyl), S, SO and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl, heteroaryl, C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;
R¹ is selected from H, halogen, C₁-C₆ alkyl, C₃-C₁₂ cycloalkyl and C₁-C₆ alkoxy;
R² is selected from C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
R³ is selected from H, halogen, C₁-C₆ alkyl and C₁-C₆ heteroalkyl;
R⁴, R⁵ and R⁶ are each independently selected from H, halogen, C₁-C₆ alkoxy, C₁-C₆ alkyl, C₃-C₁₂ cycloalkyl and C₃-C₁₂ heterocyclyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C;
R⁹ is absent when Y² is N or is selected from H, halogen and C₁-C₆ alkyl when Y² is C.

30. The compound of formula (I) for use according to claim 27, wherein
X is selected from CH₂, CF₂, CHF, CO, CHOH, CHO(C₁-C₃) alkyl, NH, N(C₁-C₃ alkyl), S, SO and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl, heteroaryl,
C₃-C₁₂ heterocyclyl, wherein the aryl, the heteroaryl and the C₃-C₁₂ heterocyclyl are optionally substituted by NH₂, C₁-C₆ alkyl, C₁-C₆ heteroalkyl, halogen, CN, C₃-C₁₂ cycloalkyl, C₃-C₁₂ heterocyclyl;
R¹ is selected from H, halogen, and C₁-C₆ alkyl;
R² is selected from C₁-C₆ alkyl, C₁-C₆ heteroalkyl, C₂-C₆ alkenyl, C₂-C₆ alkynyl, C₃-C₁₂ cycloalkyl, C₃-C₁₂ heterocyclyl, aryl and heteroaryl, wherein the aryl and the heteroaryl are optionally substituted by C₁-C₆ alkyl, C₁-C₆ heteroalkyl, halogen, CN, C₃-C₁₂ heterocyclyl, C₃-C₁₂ cycloalkyl;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C;
R⁹ is absent when Y² is N or is selected from H, halogen and C₁-C₆ alkyl when Y² is C.

31. The compound of formula (I) for use according to anyone of claims 27-30, wherein
X is selected from CH₂, CO, CHOH, CHO(C₁-C₃) alkyl, NH and O.

32. The compound of formula (I) for use according to claim 27, wherein
X is selected from CH₂, CO, CHOH, CHO(C₁-C₃) alkyl, NH and O;
Y¹, Y², and Y³ are each independently selected from N and C;
Z is NR¹⁰R¹¹;
R¹⁰ is H;
R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C; and
R⁹ is absent when Y² is N or is selected from H, halogen and C₁-C₆ alkyl when Y² is C.

33. The compound of formula (I) for use according to claim 27, wherein
X is selected from CH₂, CO, CHOH, CHO(C₁-C₃) alkyl, NH and O;
Y¹ is selected from N and C, Y² is selected from N and C and Y³ is C;
Z is NR¹⁰R¹¹;
R¹⁰ is H;
R¹¹ is selected from C₁-C₃ cyanoalkyl and C₁-C₆ alkyl substituted by aryl or heteroaryl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is selected from H, halogen and C₁-C₆ alkyl; and
R⁹ is absent when Y² is N or is selected from H, halogen and C₁-C₆ alkyl when Y² is C.

34. The compound of formula (I) for use according to claim 27, wherein
X is selected from CH₂, CO, CHOH, CHO(C₁-C₃) alkyl, NH and O;
wherein Y¹, Y², and Y³ are each independently selected from N and C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is absent when Y³ is N or is selected from H, halogen and C₁-C₆ alkyl when Y³ is C; and
R⁹ is absent when Y² is N or is selected from H, halogen and C₁-C₆ alkyl when Y² is C.

35. The compound of formula (I) for use according to claim 27, wherein
X is selected from CH₂, CO, CHOH, CHO(C₁-C₃) alkyl, NH and O;
Y¹ is selected from N and C, Y² is selected from N and C and Y³ is C;
Z is NR¹⁰R¹¹;
R¹⁰ is H;
R¹¹ is selected from C₁-C₃ cyanoalkyl, and C₁-C₆ alkyl substituted by aryl or heteroaryl, wherein the aryl is phenyl and wherein the heteroaryl is selected from pyridyl, pyrimidinyl, imidazolyl, oxadiazolyl and tetrazolyl, wherein the aryl and the heteroaryl is optionally substituted by NH₂, C₁-C₆ alkyl, C₁-C₆ heteroalkyl, halogen;
R¹ is selected from H, halogen and C₁-C₆ alkyl;
R² is selected from C₁-C₆ alkyl, C₃-C₁₂ cycloalkyl, aryl and heteroaryl, wherein the aryl and the heteroaryl are optionally substituted by C₁-C₆ alkyl, C₁-C₆ heteroalkyl, halogen;
R³ is selected from H, halogen and C₁-C₆ alkyl;
R⁴, R⁵ and R⁶ are each independently selected from H, halogen and C₁-C₆ alkyl;
R⁷ is absent when Y¹ is N or is selected from H, halogen and C₁-C₆ alkyl when Y¹ is C;
R⁸ is selected from H, halogen and C₁-C₆ alkyl; and
R⁹ is absent when Y² is N or is selected from H, halogen and C₁-C₆ alkyl when Y² is C.

36. The compound of formula (I) for use according to claim 27, wherein the compound is a compound according to anyone of claims 1 to 26.

37. The compound of formula (I) for use according to anyone of claims 27-36, wherein the oncovirus induced cancer is selected from the group consisting of angio-immunoblastic T cell lymphomas, T/NK cell lymphomas, Burkitt lymphoma, classical Hodgkin lymphoma, post-transplant lymphoproliferative disorder (PTLD), non-Hodgkin lymphoma (NHL), Nasopharyngeal Carcinoma (NPC), lympho-epithelioma like gastric carcinomas, gastric adenocarcinomas, leiomypsarcomas, X-linked lymphoproliferative dieases, AIDS related lymphoproliferative disease, AIDS-related Kaposi's Sarcoma (KS), classical Kaposi's Sarcoma, Primary Effusion Lymphoma (PEL), Multicentric Castleman's disease (MCD).

38. A pharmaceutical composition comprising a compound of formula (I) according to any one of claims 1-26 and a pharmaceutically acceptable carrier.
